(19) United States
(12) Patent Application Publication
(10) Pub. No.: US 2023/0279469 A1
(43) Pub. Date: Sep. 7, 2023

(54) FLOW CELLS AND METHODS

(71) Applicants: ILLUMINA CAMBRIDGE LIMITED, Cambridge (GB); ILLUMINA, INC., San Diego, CA (US); Illumina Singapore Pte. Ltd., Singapore (SG)

(72) Inventors: Johan Sebastian Basuki, Singapore (SG); Jonathan Mark Boutell, Bishops Stortford (GB); Jeffrey S. Fisher, San Diego, CA (US); Louise Jane Fraser, Cambridge (GB); Wayne N. George, Ilford (GB); Niall Anthony Gormley, Cambridge (GB); David Jones, Huntingdon (GB); Xiaoyu Ma, San Diego, CA (US); Maria Ines Martins Vitoriano, Baldock (GB); Zhong Mei, San Diego, CA (US); Oliver Jon Miller, Cambridge (GB); Andrew Price, San Mateo, CA (US); Sebastien Georg Gabriel Ricoult, Cambridge (GB); Vicki S. Thomson, Cambridge (GB); Jacqueline C. Weir, Cambridge (GB); Xiaoy Ma, San Diego, CA (US); Weihua Chang, San Diego, CA (US); Hui Han, San Diego, CA (US)

(21) Appl. No.: 18/146,320

(22) Filed: Dec. 23, 2022

Related U.S. Application Data
(63) Continuation of application No. PCT/US2022/082280, filed on Dec. 22, 2022.
(60) Provisional application No. 63/380,878, filed on Oct. 25, 2022.
(60) Provisional application No. 63/371,165, filed on Aug. 11, 2022.
(60) Provisional application No. 63/323,890, filed on Mar. 25, 2022.
(60) Provisional application No. 63/293,494, filed on Dec. 23, 2021.

(51) Int. Cl. C12Q 1/6806; C12N 15/10; B01L 3/00

(52) U.S. Cl. CPC C12Q 1/6806; B01L 3/502761; C12N 15/1065; B01L 2200/0647; B01L 2300/0893

(57) ABSTRACT

An example of a flow cell includes a substrate having depressions separated by interstitial regions. First and second primers are immobilized within the depressions. First transposome complexes are immobilized within the depressions, and the first transposome complexes include a first amplification domain. Second transposome complexes are also immobilized within the depressions, and the second transposome complexes include a second amplification domain. Some of the first transposome complexes, or some of the second transposome complexes, or some of both of the first and second transposome complexes include a modification to reduce tagmentation efficiency.

### CROSS-REFERENCE TO RELATED APPLICATIONS

This application is a continuation of International Application No. PCT/US2022/082280, filed Dec. 22, 2022, which itself claims the benefit of U.S. Provisional Pat. Application Serial No. 63/293,494, filed Dec. 23, 2021 and U.S. Provisional Pat. Application Serial No. 63/323,890, filed Mar. 25, 2022 and U.S. Provisional Pat. Application Serial No. 63/371,165, filed Aug. 11, 2022 and U.S. Provisional Pat. Application Serial No. 63/380,878, filed Oct. 25, 2022, each of which is incorporated herein by reference in its entirety.

### REFERENCE TO SEQUENCE LISTING

The Sequence Listing submitted herewith is hereby incorporated by reference in its entirety. The name of the file is ILI228BUS_IP-2481-US_Sequence_Listing.xml, the size of the file is 12,571 bytes, and the date of creation of the file is Dec. 23, 2022.

### BACKGROUND

Double-stranded DNA (dsDNA) target molecules can be fragmented and tagged to generate a library of smaller, single-stranded DNA molecules (ssDNA). These smaller, single-stranded DNA molecules may be used as templates in DNA sequencing reactions. The templates may enable short read lengths to be obtained, and then during data analysis, overlapping short sequence reads can be aligned to reconstruct the longer nucleic acid sequences. Some methods for fragmentation and tagging of double-stranded DNA generate excessive waste, involve expensive instruments for fragmentation, and are time-consuming.

Prior to fragmentation and tagging, the dsDNA target molecules may be extracted from a whole blood sample, bone marrow aspirate, a tissue sample, blood spots, or saliva. Some extraction processes utilize one or more reagents that inhibit enzymatic reactions that take place during fragmentation and/or tagging. Thus, the extracted dsDNA target molecules may be exposed to a purification process before being exposed to additional preparation techniques.

### SUMMARY

Some flow cells are disclosed herein that include transposome complexes and primers bound to the surface within the flow cell. Other flow cells are disclosed herein that enable a first type of transposome complex to be assembled on a first portion of the flow cell and a second type of transposome complex (e.g., a pre-assembled transposome complex) to be attached to a second portion of the flow cell. With these other flow cells, primers are bound to both the first and second portions. As part of the surface chemistry of each of these flow cells, the transposome complexes and primers enable DNA sample tagmentation and extension amplification to take place on the flow cell surface, thus eliminating the need for off flow cell library preparation (e.g., off flow cell DNA sample tagmentation).

Some of the flow cells disclosed herein include transposome complexes that are asymmetrically attached to the surface within the flow cell. By “asymmetrically attached,” is it meant that one transposome complex is attached to the flow cell surface through its 5′ end and the other transposome complex is attached to the flow cell through its 3′ end. In these examples, the ratio of the asymmetrically attached transposome complexes is skewed so that more of the 3′ end attached transposome complexes are present. The asymmetric attachment and the ratio reduce the occurrence of fragmented strands that have the same amplification domain at both the 3′ and the 5′ ends as a result of extension amplification. Reducing the occurrence of these particular fragmented strands can increase the percentage of reads passing filter (i.e., the metric used to describe clusters which pass a chastity threshold and are used for further processing and analysis of sequencing data).

Still other examples of the flow cells disclosed herein include only one type of transposome complex attached to the surface within the flow cell. With these examples, second tagmentation steps can be formed off of the flow cell or by introducing an additional transposome complex to the flow cell at some point in the workflow.

Methods for binding the transposome complexes to the flow cell surface are also disclosed herein. These methods are efficient, and can be performed during flow cell manufacturing or as part of the initial set up of the flow cell.

Also disclosed herein are methods to increase the insert size of the tagmented DNA sample. Larger inserts help when aligning reads back to the reference, as the forward strand reads and reverse strand reads (e.g., read 1 and read 2) will be further apart. Larger inserts also reduce the chance of read 1 and read 2 overlapping, which can help with GC bias and improve sequencing accuracy of otherwise tricky regions (e.g., short tandem repeats (STRs), homopolymers, etc.).

After tagmentation is performed, a chaotropic agent may be introduced to remove a transposase from the transposome complex. Some of the methods disclosed herein involve the introduction of a chelator of the chaotropic agent after the introduction of the chaotropic agent. The chelator can sequester the chaotropic agent, thus preventing it from denaturing enzymes (e.g., ligase, polymerase) that are used in downstream processes, such as extension reactions and amplification reactions.

An additional method is disclosed herein for extracting DNA from a sample selected from the group consisting of a whole blood sample, bone marrow aspirate, a tissue sample, blood spots, or saliva. This method utilizes a specific lysis buffer to extract the DNA, as well as the previously mentioned chelator to sequester a chaotropic agent in the lysis buffer. This method generates a complexed crude lysate, which can be used in a variety of library preparation methods, including the on flow cell DNA sample tagmentation disclosed herein, without first having to be exposed to a purification process. Thus, this method may reduce the amount of time involved between extracting DNA to generating library fragments from the extracted DNA.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a schematic illustration of one depression of one example of a flow cell disclosed herein including transposome complexes, in dimer form, grafted to a polymeric hydrogel;

FIG. 1B is a schematic illustration of one depression of one example of a flow cell disclosed herein including single transposome complexes grafted to a polymeric hydrogel;

FIG. 1C is a schematic illustration of the depression of FIG. 1B after a DNA sample has undergone tagmentation;

FIG. 1D is a schematic illustration of the depression of FIG. 1C after transposase removal and an extension reaction;

FIG. 2A is a schematic illustration of one depression of another example of a flow cell disclosed herein including transposome complexes bound to the polymeric hydrogel through a biotin-containing linker;

FIG. 2B is a schematic illustration of one depression of still another example of a flow cell disclosed herein including transposome complexes and primers bound to the polymeric hydrogel through biotin-containing linkers;

FIG. 3A is a schematic illustration of one depression of an example of a flow cell disclosed herein including spatially separated transposome complexes and primers;

FIG. 3B is a schematic illustration of one multi-depth depression of an example of a flow cell disclosed herein including spatially separated transposome complexes and primers;

FIG. 4 is a schematic illustration of one depression of another example of a flow cell disclosed herein including spatially separated transposome complexes and primers;

FIG. 5 is a schematic illustration of one depression of still another example of a flow cell disclosed herein including spatially separated transposome complexes and primers;

FIG. 6 is a graph depicting the percentage of reads (Y axis) having the insert size (base pairs) set forth on the X axis after tagmentation of a control and an example when spermidine is added with the DNA sample;

FIG. 7 is a graph depicting the percentage of reads (Y axis) having the insert size (base pairs) set forth on the X axis after tagmentation of a control (using non-modified transposome complexes) and an example when the transposome complexes are modified by removing the 5′ phosphate from the non-transferred stands;

FIG. 8 is a graph depicting the percentage of reads (Y axis) having the insert size (base pairs) set forth on the X axis after tagmentation of a control (Illumina PCR-free DNA preparation in a tube), and four different examples when the transposome complexes are modified by i) removing the 5′ phosphate from and adding a 3′ dye to the non-transferred stands, ii) adding the 3′ dye to the non-transferred stands, and iii) removing the 5′ phosphate from the non-transferred stands;

FIG. 9 is a graph depicting the percentage of reads (Y axis) having the insert size (base pairs) set forth on the X axis after tagmentation of a control (using non-modified transposome complexes) and five different examples when the transposome complexes are modified by adding a 3′ dideoxycytosine to the transferred stands;

FIG. 10 is a graph depicting i) the insert size (base pairs), ii) the percentage of Q30 bases, iii) the percentage of mapped reads, iv) coverage, and v) the percentage of reads passing filter (%PF) for library fragments generated using two different control methods and for library fragments generated from a whole blood sample using an example method disclosed herein;

FIG. 11A, FIG. 11B, FIG. 11C, and FIG. 11D each depict an automated electrophoresis gel image (reproduced in black and white) illustrating the effect that betacyclodextrin has on sodium dodecyl sulfate (SDS) during a polymerase chain reaction (PCT) with two different polymerases;

FIG. 12 is a graph depicting the quantitative polymerase chain reaction (qPCR) yield (nM, Y axis) versus the percentage of betacyclodextrin used in a gap fill ligation step for two different controls samples and example samples;

FIG. 13A is a schematic illustration of one depression of one example of a flow cell disclosed herein including transposome complexes asymmetrically attached within the depression;

FIG. 13B is a schematic illustration of the depression of FIG. 13A after a DNA sample has undergone tagmentation;

FIG. 13C is a schematic illustration of the depression of FIG. 13B after transposase removal and before an extension reaction;

FIG. 13D is a schematic illustration of the depression of FIG. 13B after dehybridization;

FIG. 14A schematically illustrates one example of the asymmetrically attached transposome complexes of FIG. 13A;

FIG. 14B schematically illustrates another example of the asymmetrically attached transposome complexes of FIG. 13A;

FIG. 15A is a schematic illustration of one depression of another example of a flow cell disclosed herein, the depression including different chemistry for assembling a first type of transposome complex on a first portion of the flow cell and for attaching a second type of transposome complex (i.e., a pre-assembled transposome complex) to a second portion of the flow cell;

FIG. 15B is a schematic illustration of the depression of FIG. 15A with the first type of transposome complex assembled on the first portion and the pre-assembled transposome complex attached to the second portion;

FIG. 15C is a schematic illustration of the depression of FIG. 15B after tagmentation of a DNA sample;

FIG. 15D is a schematic illustration of the depression of FIG. 15C after transposase removal;

FIG. 15E is a schematic illustration of the depression of FIG. 15D after a strand displacement reaction;

FIG. 15F is a schematic illustration of the depression of FIG. 15E after washing;

FIG. 15G is a schematic illustration of the depression of FIG. 15F after hybridization;

FIG. 15H is a schematic illustration of the depression of FIG. 15G after polymerase extension;

FIG. 15I is a schematic illustration of the depression of FIG. 15H after cleaving of orthogonally cleavable linkages;

FIG. 15J is a schematic illustration of the depression of FIG. 15I after clustering;

FIG. 16A schematically illustrates one example of the assembled transposome complex of FIG. 15B;

FIG. 16B schematically illustrates one example of the pre-assembled transposome complex) of FIG. 15B;

FIG. 17 is a schematic view of an example primer set that can be used in some examples of the flow cell disclosed herein;

FIG. 18 is a bar graph depicting the DNA yield for blood samples manually extracted or extracted via an automated process, where Proteinase K was added to a lysis buffer or was pre-dried on a sample collection tip;

FIG. 19 is a graph depicting i) the insert size (base pairs), ii) the percentage of Q30 bases, iii) the percentage of mapped reads, iv) coverage, and v) the percentage of reads passing filter (%PF) for library fragments generated from a whole blood sample using three example methods disclosed herein;

FIG. 20 is a graph depicting i) the insert size (base pairs), ii) the percentage of Q30 bases, iii) the percentage of mapped reads, iv) coverage, and v) the percentage of reads passing filter (%PF) for library fragments generated from a DNA sample using flow cells prepared with liquid streptavidin and lyophilized and reconstituted streptavidin;

FIG. 21 is a graph depicting i) the insert size (base pairs), ii) the percentage of Q30 bases, iii) the percentage of mapped reads, iv) coverage, and v) the percentage of reads passing filter (%PF) for library fragments generated from a whole blood sample prepared with a liquid lysis buffer and a lyophilized and reconstituted lysis buffer;

FIG. 22 is a graph depicting i) the insert size (base pairs), ii) the percentage of Q30 bases, iii) the percentage of mapped reads, iv) coverage, and v) the percentage of reads passing filter (%PF) for library fragments generated from a DNA sample prepared in a liquid tagmentation buffer and a lyophilized and reconstituted tagmentation buffer;

FIG. 23 is a graph depicting i) the insert size (base pairs), ii) the percentage of Q30 bases, iii) the percentage of mapped reads, iv) the percentage of reads passing filter (%PF) for library fragments, and v) coverage generated from a DNA sample prepared and sequenced using a commercially available NEXTSEQ™ 2 K sequencing kit and an example workflow described herein;

FIG. 24 is a schematic illustration of an example of a tagmentation kit;

FIG. 25 is a graph depicting i) the insert size (base pairs), ii) occupancy or %occupied, iii) the percentage of Q30 bases, iv) the percentage of mapped reads, and v) the percentage of reads passing filter (%PF) for library fragments generated from a DNA sample prepared and sequenced using flow cells whose linking chemistry (BCN-biotin-streptavidin) was introduced via different methods;

FIG. 26A through FIG. 26D are schematic illustrations that together depict an example method, where FIG. 26A depicts a multi-depth depression with the polymeric hydrogel introduced thereto, FIG. 26B depicts the introduction of a first transposome and primer set to one portion of the multi-depth depression while the other portion of the multi-depth depression is masked with an insoluble photoresist, FIG. 26C depicts the introduction of a second transposome and primer set to the other portion of the multi-depth depression, and FIG. 26D depicts tagmentation across the multi-depth depression;

FIG. 27 is a schematic flow diagram illustrating different example methods that begin with one type of transposome complex in a flow cell depression; and

FIG. 28 is a graph depicting i) the insert size (base pairs), ii) the occupancy or %occupied, iii) the percentage of Q30 bases, iv) the percentage of mapped reads, and v) the percentage of reads passing filter (%PF) for library fragments generated from a DNA sample prepared sequenced using flow cells whose linking chemistry (BCN-biotin-streptavidin) was introduced via different methods.

### DETAILED DESCRIPTION

Examples of the flow cells disclosed herein include transposome complexes immobilized to the surface of the flow cell. By incorporating the transposome complexes on the flow cell surface, tagmentation of a DNA sample can take place on the flow cell surface. On flow cell tagmentation generates DNA sample fragments (or library fragments of the larger DNA sample) on the same surface where amplification and sequencing of the fragments takes place. This eliminates the need for off flow cell DNA sample preparation to generate the library fragments, and thus provides a more stream-lined and efficient process.

In some examples, the transposome complexes and/or the reagents used in tagmentation are modified in order to increase the insert size of the fragmented products, which may be desirable.

In some of the methods disclosed herein, a chelator of the chaotropic agent is used to sequester the chaotropic agent after tagmentation is performed. Sequestration in the manner described herein keeps the chaotropic agent from denaturing enzymes (e.g., ligase, polymerase) that are used in downstream processes, and also eliminates the need for washes after tagmentation. If a bead-based tagmentation process is used, sequestration in the manner described herein also eliminates the need for magnets to separate the beads from the tagmentation buffer.

In some examples, the DNA sample used to generate the DNA sample fragments is prepared from a whole blood sample in a manner that allows it to be exposed directly to library preparation without first being exposed to purification.

In still other examples, the transposome complexes used in tagmentation are asymmetrically attached in order to reduce the occurrence of fragment strands that have the same amplification domain at both the 3′ and the 5′ ends.

### Definitions

Terms used herein will be understood to take on their ordinary meaning in the relevant art unless specified otherwise. Several terms used herein and their meanings are set forth below.

As used herein, the singular forms “a,” “an,” and “the” refer to both the singular as well as plural, unless the context clearly indicates otherwise. The term “comprising” as used herein is synonymous with “including,” “containing,” or “characterized by,” and is inclusive or open-ended and does not exclude additional, unrecited elements or method steps.

Reference throughout the specification to “one example,” “another example,” “an example,” and so forth, means that a particular element (e.g., feature, structure, composition, configuration, and/or characteristic) described in connection with the example is included in at least one example described herein, and may or may not be present in other examples. In addition, it is to be understood that the described elements for any example may be combined in any suitable manner in the various examples unless the context clearly dictates otherwise.

The terms “substantially” and “about” used throughout this disclosure, including the claims, are used to describe and account for small fluctuations, such as due to variations in processing. For example, these terms can refer to less than or equal to ±5% from a stated value, such as less than or equal to ±2% from a stated value, such as less than or equal to ±1 % from a stated value, such as less than or equal to ±0.5% from a stated value, such as less than or equal to ±0.2% from a stated value, such as less than or equal to ±0.1% from a stated value, such as less than or equal to ±0.05% from a stated value.

Adapter: A linear oligonucleotide sequence that can be fused to a nucleic acid molecule, for example, by ligation or tagmentation. Suitable adapter lengths may range from about 10 nucleotides to about 100 nucleotides, or from about 12 nucleotides to about 60 nucleotides, or from about 15 nucleotides to about 50 nucleotides. The adapter may include any combination of nucleotides and/or nucleic acids. In some examples, the adapter can include an amplification domain, e.g., having a universal nucleotide sequence, such as a P5 or P7 sequence, that can serve as a starting point for template amplification and cluster generation. In other examples, the adapter can include a sequence that is complementary to at least a portion of a flow cell surface bound primer (which includes the universal nucleotide sequence. In the latter example, the adapter sequence can hybridize to the complementary flow cell surface bound primer during amplification and cluster generation. In some examples, the adapter can also include a sequencing primer sequence (i.e., sequencing binding site) or a sequencing sample index (i.e., a barcode sequence). Combinations of different adapters may be incorporated into the nucleic acid molecule, such as the DNA fragments generated via tagmentation.

Amplification Domain: A portion of an adapter having a universal nucleotide sequence, such as a P5 or P7 sequence, that can serve as a starting point for template amplification and cluster generation.

Asymmetrically attached: Within a group of transposome complexes, some are attached to the flow cell surface through a 3′ end of a non-transferred strand and some others are attached to the flow cell surface through a 5′ end of a transferred strand.

Complexed Crude Lysate: A product generated by exposing a sample selected from the group consisting of a whole blood sample, bone marrow aspirate, a tissue sample, blood spots, and saliva to i) an inorganic salt free lysis buffer, ii) inactivation of the protease contained in the inorganic salt free lysis buffer, and iii) a chelator of a chaotropic detergent in the inorganic salt free lysis buffer. This product can be used in downstream library preparation techniques without first being exposed to a purification method.

Depositing: Any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, screen printing, microcontact printing, inkjet printing, or the like.

Depression: A discrete concave feature in a substrate or a layer of a substrate (e.g., a patterned resin) having a surface opening that is at least partially surrounded by interstitial region(s) of the substrate or the layer. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc. The depression may also have more complex architectures, such as ridges, step features, etc.

DNA Sample: A polymeric form of nucleotides of any length that includes deoxyribonucleotides, deoxyribonucleotide analogs, or complementary deoxyribonucleotides derived from an RNA (ribonucleic acid) sample. The DNA sample is double stranded. The DNA sample may include naturally occurring DNA, which includes a nitrogen containing heterocyclic base (a nucleobase such as adenine, thymine, cytosine and/or guanine), a sugar (specifically deoxyribose, i.e., a sugar lacking a hydroxyl group that is present at the 2′ position in ribose), and a backbone containing phosphodiester bonds. An analog structure can have an alternate backbone linkage including any of a variety known in the art.

The DNA sample may be genomic DNA (gDNA) that can be isolated from one or more cells, bodily fluids (e.g., whole blood, blood spots, saliva) or tissues. gDNA can be prepared by lysing a cell that contains the DNA. The cell may be lysed under conditions that substantially preserve the integrity of the cell’s gDNA. In one particular example, thermal lysis may be used to lyse a cell. In another particular example, exposure of a cell to alkaline pH can be used to lyse a cell while causing relatively little damage to gDNA. Any of a variety of basic compounds can be used for lysis including, for example, potassium hydroxide, sodium hydroxide, and the like. Additionally, relatively undamaged gDNA can be obtained from a cell lysed by an enzyme that degrades the cell wall. Cells lacking a cell wall either naturally or due to enzymatic removal can also be lysed by exposure to osmotic stress. Other conditions that can be used to lyse a cell include exposure to detergents, mechanical disruption, sonication heat, pressure differential such as in a French press device, or Dounce homogenization. Agents that stabilize gDNA can be included in a cell lysate or isolated gDNA sample including, for example, nuclease inhibitors, chelating agents, salts, buffers and the like. A crude cell lysate containing gDNA may be used without further isolation of the gDNA. In one example, a whole blood sample may be lysed using an inorganic salt free lysis buffer, and the crude lysate may be exposed to specific processing steps to generate a complexed crude lysate as defined herein. This complexed crude lysate can also be used as the DNA sample without further isolation or purification.

Each: When used in reference to a collection of items, each identifies an individual item in the collection, but does not necessarily refer to every item in the collection. Exceptions can occur if explicit disclosure or context clearly dictates otherwise.

Flow Cell: A vessel having a flow channel where a reaction can be carried out, an inlet for delivering reagent(s) to the chamber, and an outlet for removing reagent(s) from the chamber. In some examples, the flow cell enables the detection of the reaction that occurs therein. For example, the flow cell can include one or more transparent surfaces allowing for the optical detection of arrays, optically labeled molecules, or the like.

Flow channel: An area defined between two bonded or otherwise attached components, which can selectively receive a liquid sample. In some examples, the flow channel may be defined between two patterned sequencing surfaces or a patterned sequencing surface and a lid, and thus may be in fluid communication with one or more components of the sequencing surface(s).

Fragment: A portion or piece of the DNA sample. A “partially adapted fragment” is a portion or piece of the DNA sample that has been tagmented, and thus includes adapter ligated to the 5′ end of the DNA fragment. A “fully adapted fragment” is a portion or piece of the DNA sample that has adapters incorporate at both the 3′ and 5′ ends of the DNA fragment.

Inorganic salt free lysis buffer: A lysis buffer that does not contain an inorganic salt, e.g., sodium salts, such as sodium chloride, sodium sulfate, and sodium carbonate; potassium salts, such as potassium chloride; and lithium salts, such as lithium chloride. The inorganic salt free lysis buffer may include organic salts, including those that function as chelating agents or detergents.

Primer: A single stranded nucleic acid molecule that can hybridize to a target sequence, such as an adapter attached to a fragment. As one example, a flow cell surface bound primer can serve as a starting point for fragment amplification and cluster generation. As another example, a primer (e.g., a sequencing primer) may be introduced that can hybridize to fragments or fragment amplicons in order to prime synthesis of a new strand that is complementary to the fragments or fragment amplicons. Any primer can include any combination of nucleotides or analogs thereof. In some examples, the primer is a single-stranded oligonucleotide or polynucleotide. The primer length can be any number of bases long. In an example, each of the flow cell surface bound primer and the sequencing primer is a short strand, ranging from 10 to 60 bases, or from 20 to 40 bases.

Tagmentation: A process in which the DNA sample is cleaved/fragmented and tagged (e.g., with the adapters) for analysis. Tagmentation is an in vitro transposition reaction.

Transferred and Non-Transferred Strands: The term “transferred strand” refers to a sequence that includes a transferred portion of a transposon end. Similarly, the term “non-transferred strand” refers to a sequence that includes the non-transferred portion of a transposon end. The 3′-end of a transferred strand is joined or transferred to a double stranded fragment during tagmentation. The non-transferred strand is not joined or transferred to the double stranded fragment during tagmentation. In an example, the transferred and non-transferred strands include at least partially complementary portions that are covalently bound together.

Transposase: An enzyme that is capable of forming a functional complex with a transposon end-containing composition (e.g., transposons, transposon ends, transposon end compositions) and catalyzing insertion or transposition of the transposon end-containing composition into the double-stranded DNA sample with which it is incubated, for example, in the in vitro transposition reaction (i.e., tagmentation). A transposase as presented herein can also include integrases from retrotransposons and retroviruses. Although many examples described herein refer to Tn5 transposase and/or hyperactive Tn5 transposase, it will be appreciated that any transposase that is capable of inserting a transposon end with sufficient efficiency to 5′-tag and fragment the DNA sample for its intended purpose can be used.

Transposome Complex: A complex formed between a transposase and a double stranded nucleic acid including a transposase integration recognition site. For example, the transposome complex can be a transposase enzyme pre-incubated with double-stranded transposon DNA under conditions that support non-covalent complex formation. Double-stranded transposon DNA can include, for example, Tn5 DNA, a portion of Tn5 DNA, a transposon end composition, a mixture of transposon end compositions or other double-stranded DNAs capable of interacting with a transposase, such as the hyperactive Tn5 transposase.

Transposon End: A double-stranded nucleic acid strand that exhibits only the nucleotide sequences (the “transposon end sequences”) that are necessary to form the complex with the transposase that is functional in tagmentation. The double-stranded nucleic acid strand of the transposon end can include any nucleic acid or nucleic acid analogue suitable for forming the functional complex with the transposase. For example, the transposon end can include natural DNA or DNA analogs (with modified bases and/or backbones), and can include nicks in one or both strands.

It is to be understood that the prime (′) designations for any of the reference numeral, e.g., primers 36′, amplification domain 26′, etc. do not refer to complementary sequences to the corresponding element primers 36, amplification domain 26, but rather are additional examples of the element. When used to describe a specific primer, e.g., P5′ or P7′, the prime (′) does refer to the complementary sequence.

### Transposome Complexes

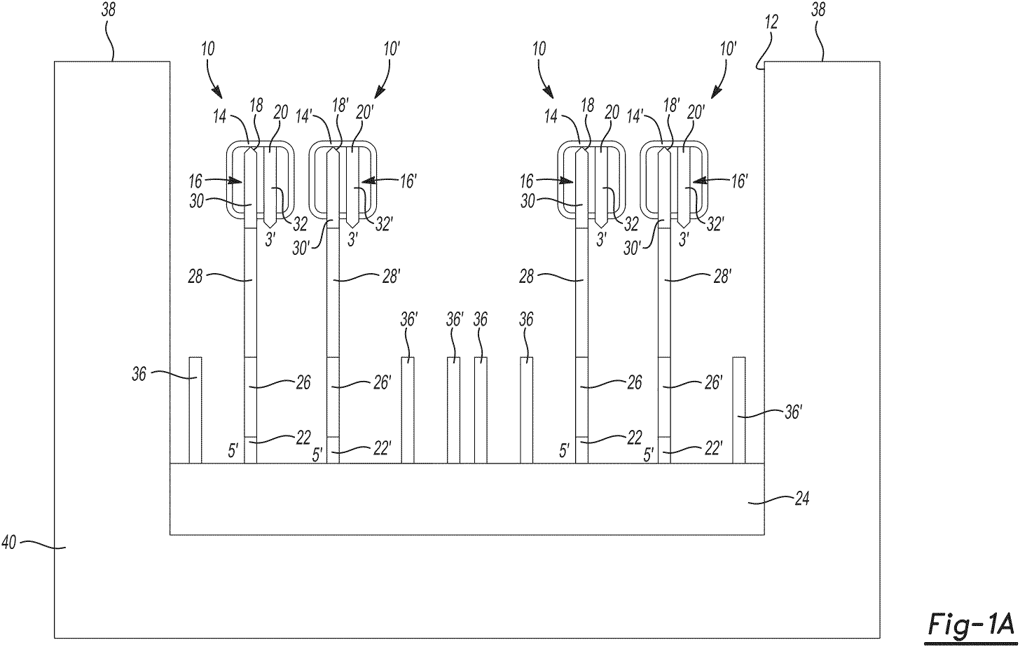

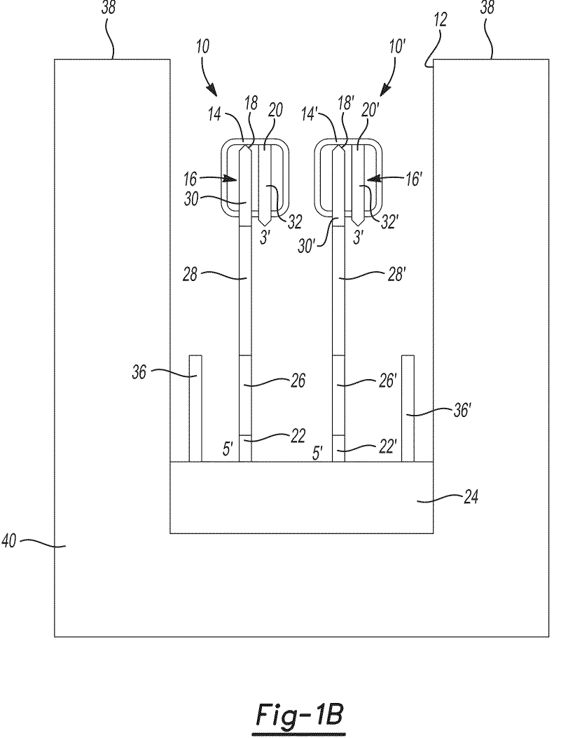

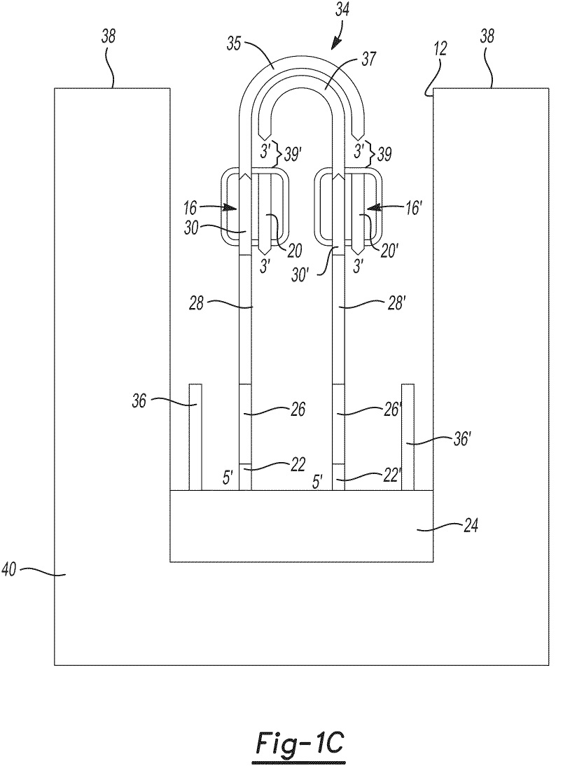

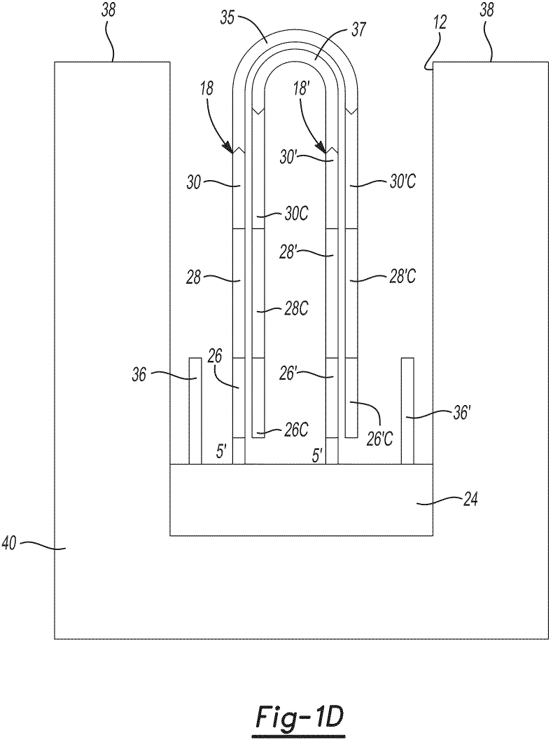

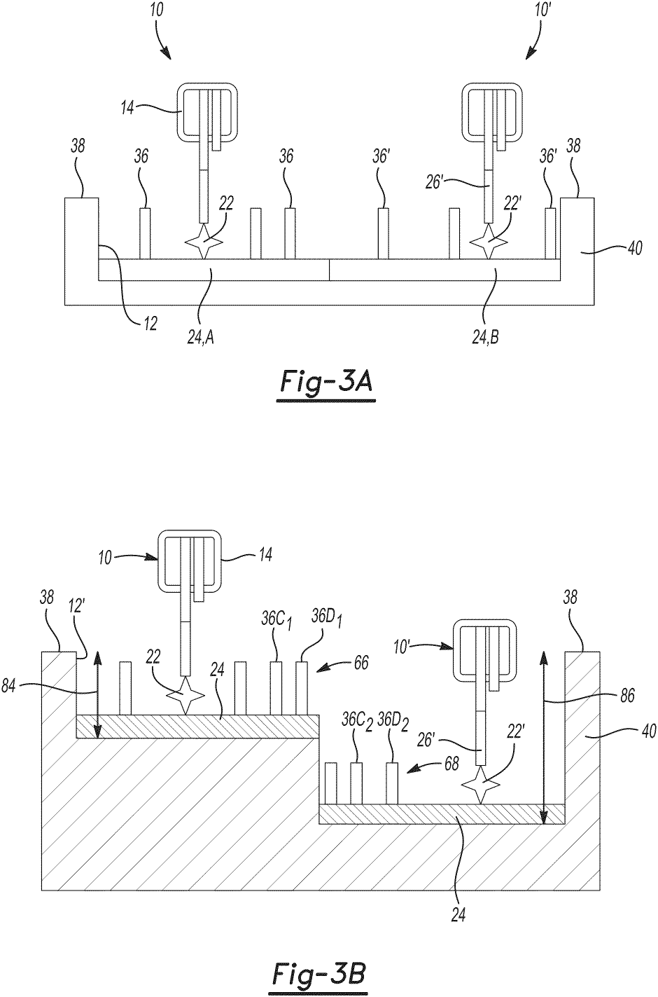

FIG. 1A, FIG. 1B, FIG. 1C, and FIG. 1D illustrate examples of some of the transposome complexes 10, 10′ disclosed herein immobilized within a flow cell depression 12. While a single set of transposome complexes 10, 10′ is shown in each of these figures, it is to be understood that several sets of transposome complexes 10, 10′ may be attached within a single depression 12. The set of transposome complexes 10, 10′ in FIG. 1A is depicted in dimer form, while the set of transposome complexes 10, 10′ shown in FIG. 1B through FIG. 1D are depicted as single transposome complexes 10, 10′. In the examples shown in FIG. 1A through FIG. 1D, the transposome complexes 10, 10′ are immobilized within a depression 12. The transposome complexes 10, 10′ may also be immobilized in a spatially separated manner, as described herein below in reference to FIG. 3A through FIG. 5.

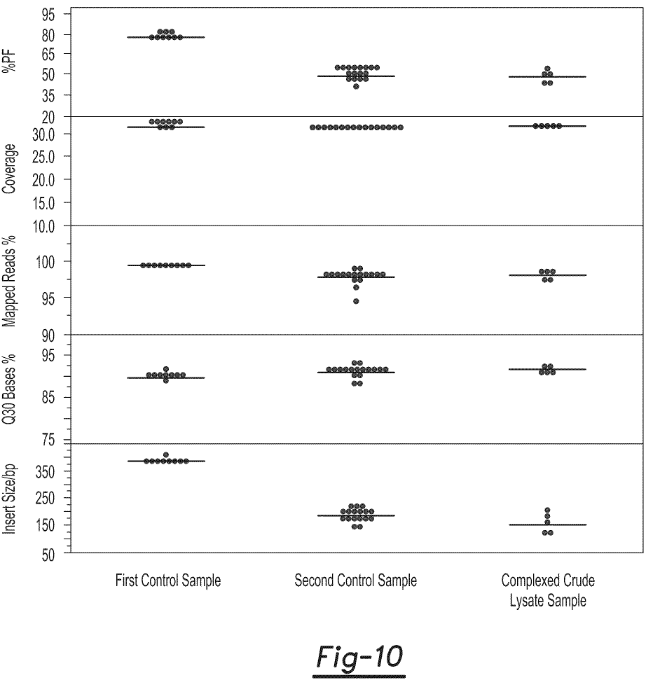

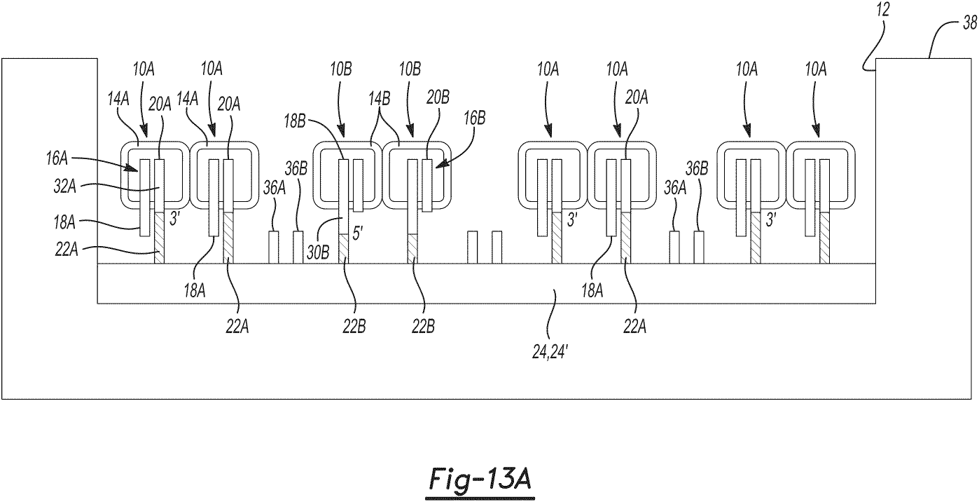

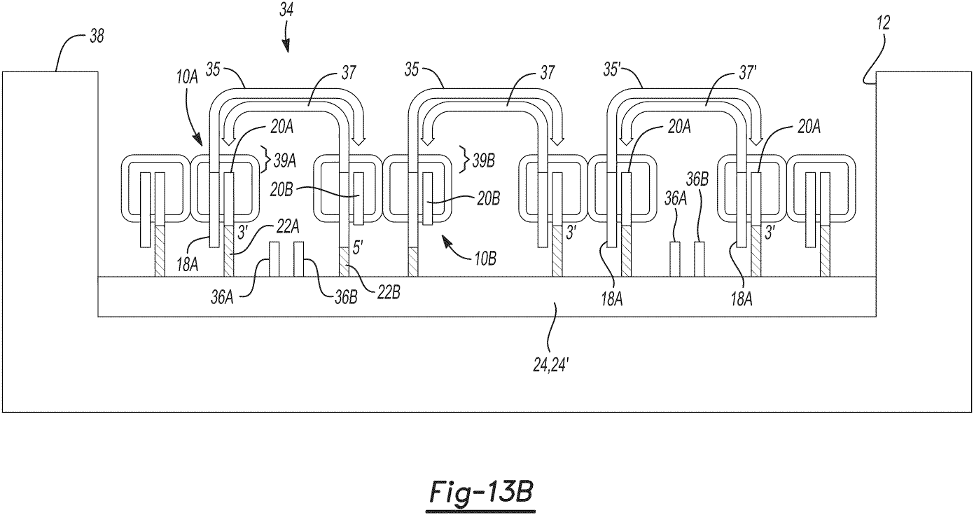

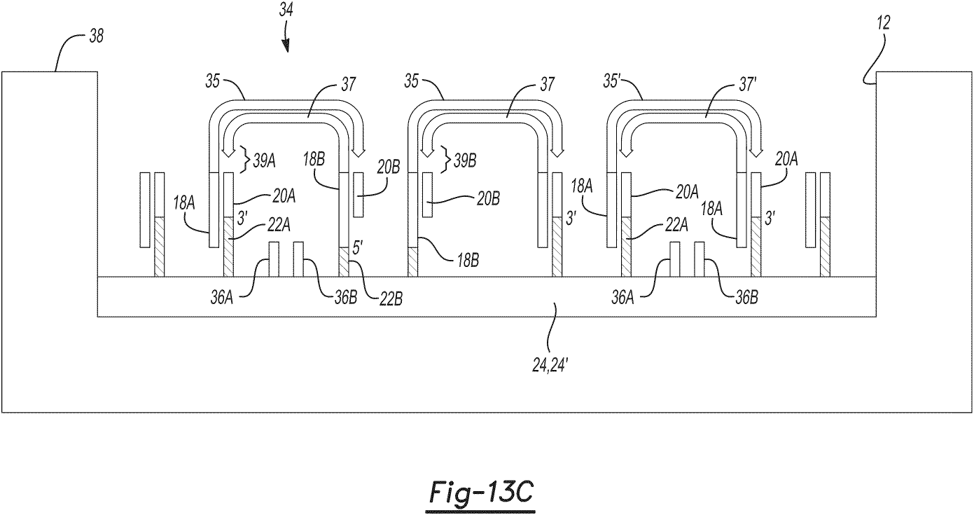

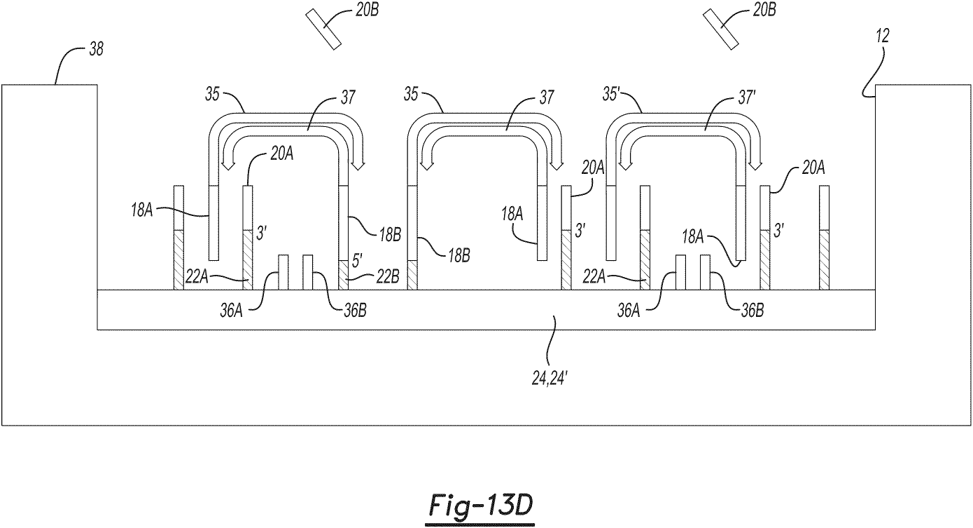

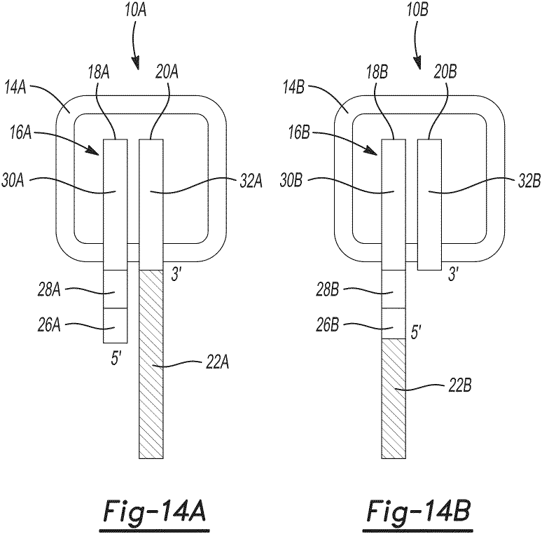

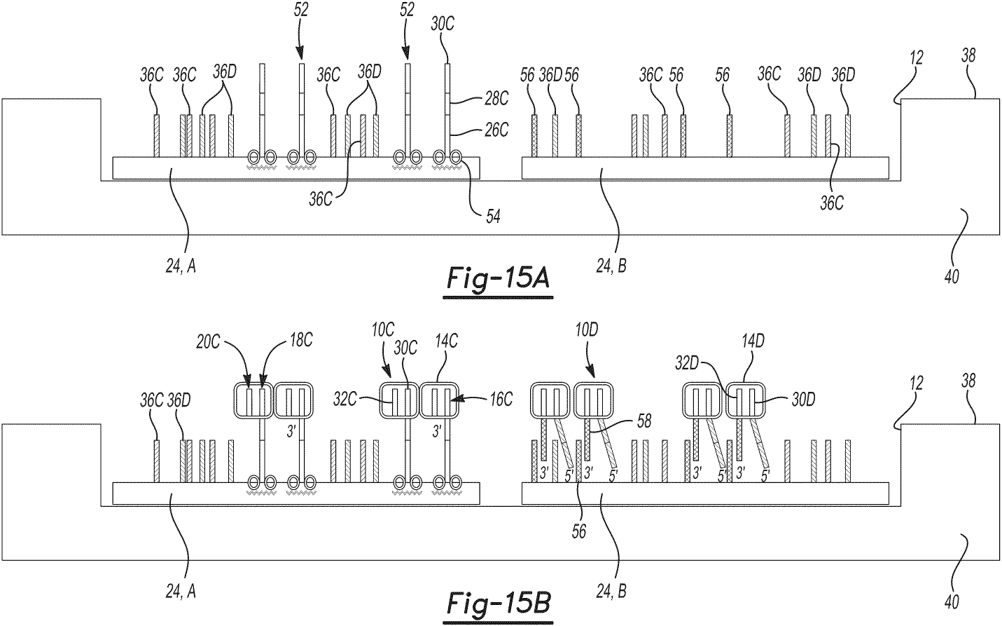

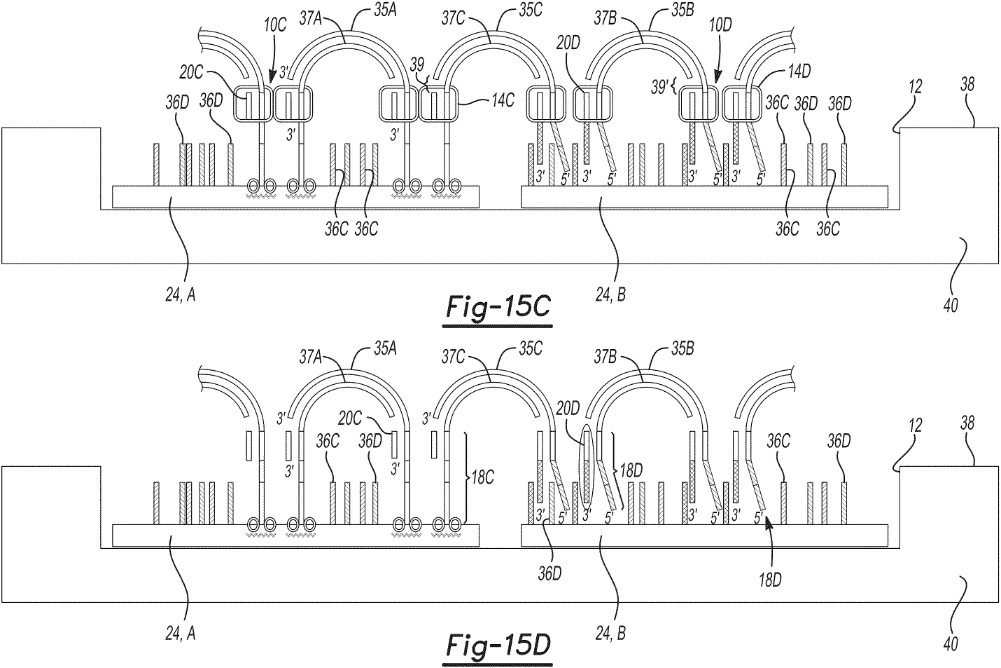

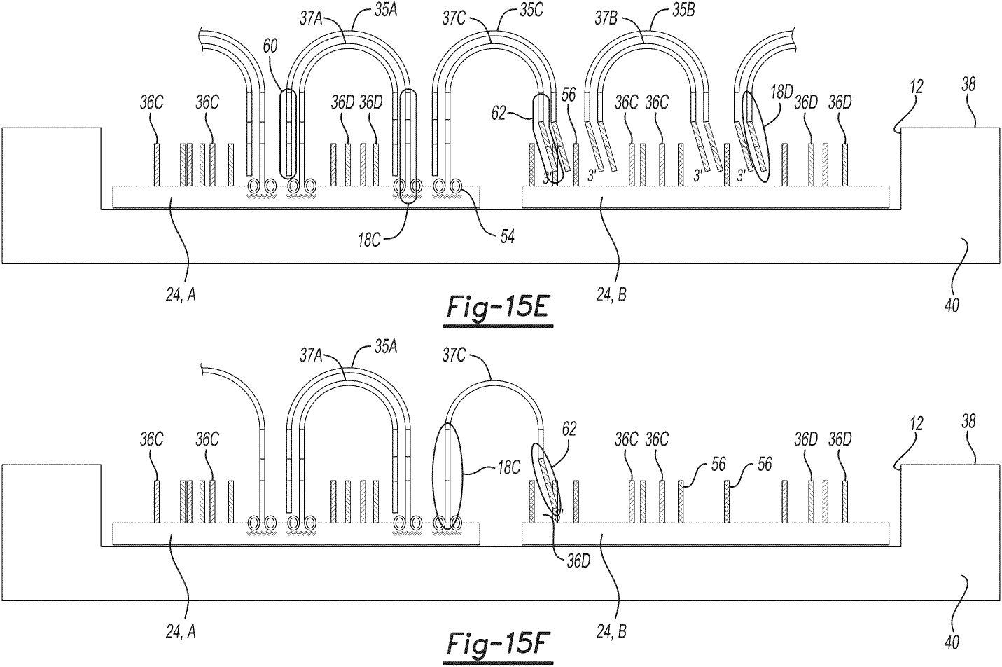

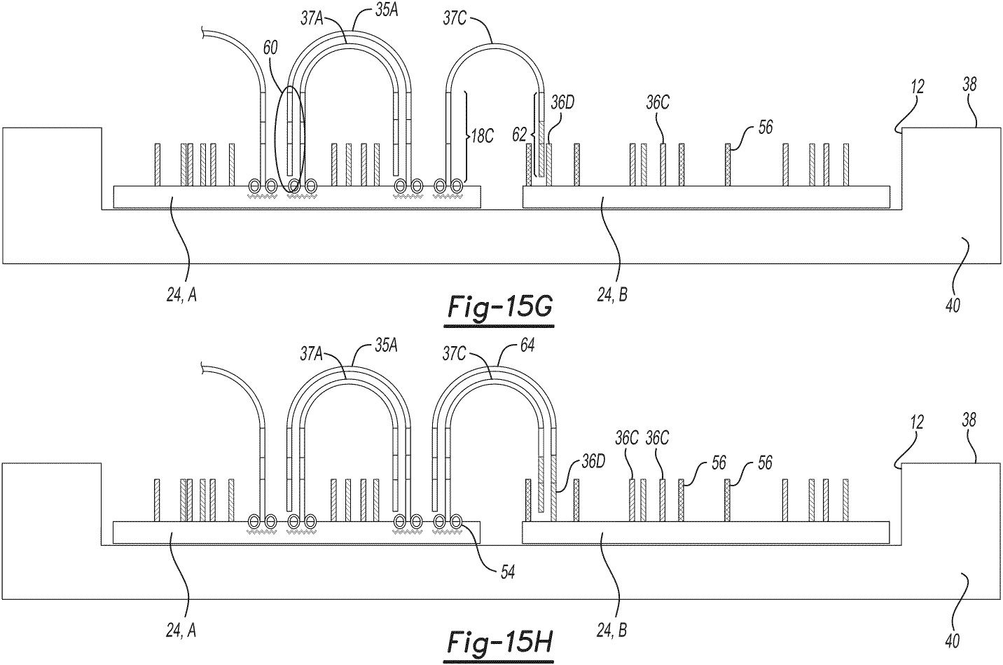

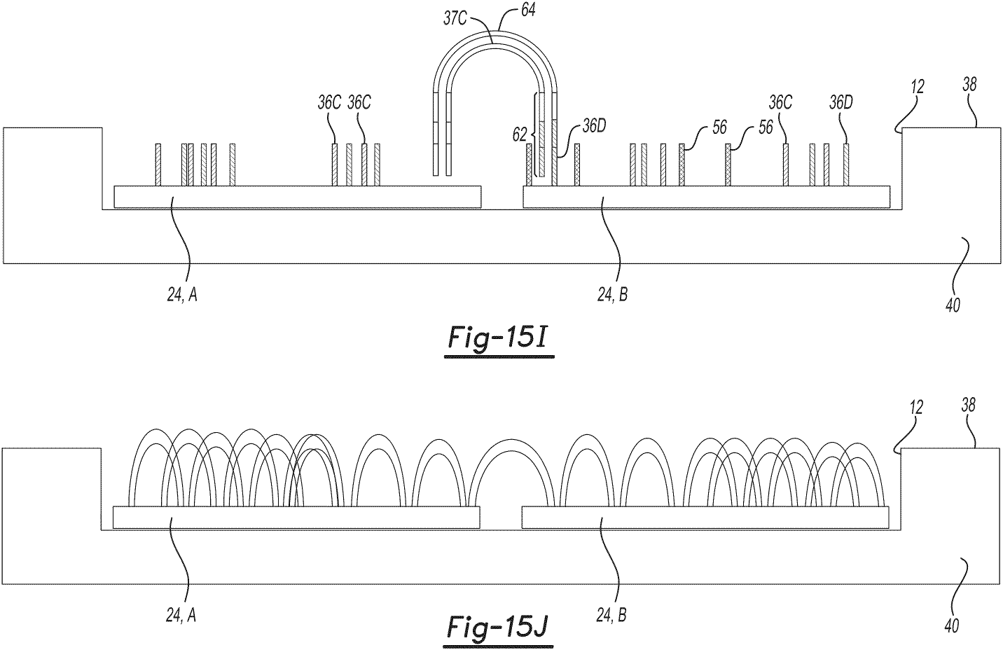

Examples of the transposome complexes 10, 10′ (or 10A, 10B described herein in reference to FIG. 13A through FIG. 13D, FIG. 14A and FIGS. 14B or 10C, 10D described in reference to FIG. 15A through FIG. 15J) are separate entities, but will form dimers in solution and when attached to the flow cell surface. It is to be understood that for simplicity, single transposome complexes are shown in FIG. 1B through FIG. 5. The dimer form of some of the transposome examples are shown, e.g., in FIG. 1A and in some figures of the FIG. 13 and FIG. 15 series (e.g., see FIG. 13A and FIG. 15B).

As mentioned and as shown in FIG. 1A and FIG. 1B, the transposome complexes 10, 10′ include a transposase enzyme 14, 14′ non-covalently bound to a transposon end 16, 16′. Each transposon end 16, 16′ is a double-stranded nucleic acid strand, one strand 30, 30′ of which is part of a transferred strand 18, 18′ and the other strand 32, 32′ of which is part of a non-transferred strand 20, 20′. In other words, the transposon end 16, 16′ includes a portion (strand 30, 30′) of the transferred strand 18, 18′ that is hybridized to a portion (e.g., strand 32, 32′) of the non-transferred strand 20, 20′.

The transferred strand 18 includes a 5′ end functional group 22 that is capable of covalently or non-covalently attaching, directly or indirectly, to surface functional groups of a polymeric hydrogel 24 in the flow cell depression 12, a first amplification domain 26, and a sequencing primer sequence 28 that is attached to one strand 30 of the transposon end 16. The strand 30 of the transposon end 16 is positioned at the 3′ end of the transferred strand 18. Similar to the transferred strand 18, the transferred strand 18′ includes a 5′ end functional group 22′ that is capable of covalently attaching to surface functional groups of a polymeric hydrogel 24 in the flow cell depression 12, a second amplification domain 26′, and a sequencing primer sequence 28′ that is attached to one strand 30′ of the transposon end 16′. The strand 30′ of the transposon end 16 is positioned at the 3′ end of the transferred strand 18′.

The 5′ end functional groups 22, 22′ may be any functional group that is capable of covalently or non-covalently attaching, directly or indirectly, to surface functional groups of a polymeric hydrogel 24, and thus will depend upon the surface functional groups of the polymer hydrogel 24. In one example, the polymeric hydrogel 24 includes azide or tetrazine surface groups, and the 5′ end functional groups 22, 22′ include a terminal alkyne (e.g., hexynyl) or an internal alkyne, where the alkyne is part of a cyclic compound (e.g., bicyclo[6.1.0]nonyne (BCN)). In another example, the polymeric hydrogel 24 is functionalized with biotin surface groups (referred to herein as biotinylated polymeric hydrogel 24′), and the 5′ end functional groups 22, 22′ also include biotin. In these examples, additional streptavidin or avidin is added to indirectly attach the biotin groups to one another.

The first and second amplification domains 26, 26′ have different sequences from each other, but have the same sequence, respectively, as first and second primers 36, 36′ attached to the polymer hydrogel 24. The first amplification domain 26 and the primer 36 together with the second amplification domain 26′ and the primer 36′ enable the amplification of the DNA sample fragments generated during tagmentation.

Examples of suitable sequences for the first amplification domain 26/primer 36 and for the second amplification domain 26′/primer 36′ include P5 and P7 primer sequences; P15 and P7 primer sequences; or any combination of the PA primer sequences, the PB primer sequences, the PC primer sequences, and the PD primer sequences set forth herein. Examples of P5 and P7 primer sequences are used on the surface of commercial flow cells sold by Illumina Inc. for sequencing, for example, on HISEQ™, HISEQX™, MISEQ™, MISEQDX™, MINISEQ™, NEXTSEQ™, NEXTSEQDX™, NOVASEQ™, ISEQ™, GENOME ANALYZER™, and other instrument platforms.

The P5 primer sequence is:

P5 #1: 5′ → 3′

AATGATACGGCGACCACCGAGAnCTACAC (SEQ. ID. NO. 1)

where “n” is uracil in the sequence.

P5 #2: 5′ → 3′

AATGATACGGCGACCACCGAGAnCTACAC (SEQ. ID. NO. 2)

where “n” is alkene-thymidine (i.e., alkene-dT) in the sequence.

The P7 primer sequence may be any of the following:

P7 #1: 5′ → 3′

CAAGCAGAAGACGGCATACGAnAT (SEQ. ID. NO. 3)

P7 #2: 5′ → 3′

CAAGCAGAAGACGGCATACnAGAT (SEQ. ID. NO. 4)

where “n” is 8-oxoguanine in each of the sequences.

The P15 primer sequence is:

P15: 5′ → 3′

AATGATACGGCGACCACCGAGAnCTACAC (SEQ. ID. NO. 5)

where “n” is allyl-T.

The other primer sequences (PA-PD) mentioned above include:

PA 5′ → 3′

GCTGGCACGTCCGAACGCTTCGTTAATCCGTTGAG (SEQ. ID. NO.
6)

PB 5′ → 3′

CGTCGTCTGCCATGGCGCTTCGGTGGATATGAACT (SEQ. ID. NO.
7)

PC 5′ → 3′

ACGGCCGCTAATATCAACGCGTCGAATCCGCAACT (SEQ. ID. NO.
8)

PD 5′ → 3′

GCCGCGTTACGTTAGCCGGACTATTCGATGCAGC (SEQ. ID. NO. 9
)

While not shown in the example sequences for PA-PD, it is to be understood that any of these sequences may include a cleavage site, such as uracil, 8-oxoguanine, allyl-T, diols, etc. at any point in the strand. The sequences for the first amplification domain 26/primer 36 and for the second amplification domain 26′/primer 36′ may be selected to have orthogonal cleavage sites (i.e., one cleavage site is not susceptible to the cleaving agent used for the other cleavage site), so that after amplification, forward or reverse strands can be cleaved, leaving the other of the reverse or forward strands for sequencing.

The primers 36, 36′ may also include a polyT sequence at the 5′ end of the primer sequence. In some examples, the polyT region includes from 2 T bases to 20 T bases. As specific examples, the polyT region may include 3, 4, 5, 6, 7, or 10 T bases.

The sequencing primer sequences 28, 28′ have different sequences from each other that respectively bind to sequencing primers introduced into the flow cell after tagmentation and amplification. As examples, the sequencing primer sequence 28 may bind a sequencing primer that primes synthesis of a new strand that is complementary to forward strand fragments/fragment amplicons and the sequencing primer sequence 28′ may bind a sequencing primer that primes synthesis of a new strand that is complementary to reverse strand fragments/fragment amplicons.

The transposon ends 16, 16′ of each transposome complex 10, 10′ include the strands 30, 30′ respectively hybridized to the strands 32, 32′. As such, the strands 30 and 32 are complementary and the strands 30′, 32′ are complementary. The double stranded transposon ends 16, 16′ are respectively capable of complexing with the transposases 14, 14′. As examples, the strands 30, 32 and 30′, 32′ of the transposon ends 16, 16′ may be the related but non-identical 19-base pair (bp) outer end (e.g., strands 30, 30′) and inner end (e.g., strands 32, 32′) sequences that serve as the substrate for the activity of the Tn5 transposase, or the mosaic ends recognized by a wild-type or mutant Tn5 transposase, or the R1 end (e.g., strands 30, 30′) and the R2 end (strands 32, 32′) recognized by the MuA transposase.

FIG. 1C illustrates the depression 12 after a DNA sample has been introduced. The DNA sample may be introduced with a tagmentation buffer, which may include water, an optional co-solvent (e.g., dimethylformamide), a metal co-factor for the transposase (e.g., magnesium acetate), and a buffer salt (e.g., Tris(hydroxymethyl) aminomethane (Tris or TRIS) acetate salt, pH 7.6). In an example, the optional co-solvent may be present in an amount up to about 11%, the metal co-factor may be present in a concentration ranging from about 3 mM to about 5.5 mM, and the buffer salt may be present in a concentration ranging from about 7 mM to about 12 mM.

When the DNA sample (shown fragmented at reference numeral 34) is introduced into the flow cell including the transposome complexes 10, 10′, the DNA sample is fragmented and the 5′ ends of both strands 35, 37 of the duplex fragments 34 are ligated to respective 3′ ends of the transferred strands 18, 18′ of the transposome complexes 10, 10′. Fragmentation and ligation may take place at a temperature at or above 30° C. In one example, the temperature may range from 30° C. to about 55° C. In another example, the temperature may range from 35° C. to about 45° C. The 3′ ends of the duplex fragments 34 are not ligated to the 5′ ends of the non-transferred strands 20, 20′. As such, a gap 39 exists between the 3′ end of the DNA fragment strand 35 and the 5′ end of the non-transferred strand 20′, and a gap 39′ exists between the 3′ end of the DNA fragment strand 37 and the 5′ end of the non-transferred strand 20. In one example, each gap 39, 39′ is nine (9) base pairs long.

As shown in FIG. 1D, the transposases 14, 14′ are then removed from the complexes 10, 10′, which are now attached to the fragments via the transferred strands 18, 18′. Transposase 14, 14′ removal may be accomplished, for example, using sodium dodecyl sulfate (SDS) or proteinase, or by heating the flow cell to about 60° C. As such, after tagmentation, some example methods involve introducing a washing solution into the flow cell; heating the flow cell, containing the washing solution, to about 60° C.; and then introducing an extension amplification mix into the flow cell. Prior to introducing the extension amplification mix, the temperature may be reduced to about 38° C.

It has been found that heating of the flow cell after tagmentation may be sufficient to denature the transposase 14, 14′, without including additional buffers or reagents. An example extension amplification mix includes a recombinase, a polymerase, and accessory proteins. An example washing solution is an aqueous solution including a buffer agent (e.g., Tris), a salt (e.g., sodium chloride, sodium citrate, etc.), a surfactant (e.g., TWEEN polysorbates), and/or a chelating agent (e.g., EDTA). In one example, the washing solution includes water, the salt at a concentration ranging from about 25 mM to about 50 mM, the surfactant in an amount ranging from about 0.01 wt% to about 0.1 wt%, and optionally the chelating agent. The washing solution may have a relatively high pH, e.g., ranging from about 7 to about 10.

When the transposase 14, 14′ removal is accomplished using sodium dodecyl sulfate (SDS) or another chaotropic detergent, the presence of this reagent can act as an inhibitor for subsequent enzymatic reactions involving, e.g., recombinases, ligases, polymerases, and the like. As such, the presence of the chaotropic detergent may deleteriously affect subsequent library preparation reactions and/or amplification reactions. In this example method, a chelator of the chaotropic detergent may be added to sequester the chaotropic detergent. The chelator that is used depends upon the chaotropic detergent that is used. In an example, the chaotropic detergent is sodium dodecyl sulfate, and the chelator of the chaotropic detergent is a cyclodextrin selected from the group consisting of alphacyclodextrin, betacyclodextrin, and methyl betacyclodextrin. An aqueous solution containing from about 1 wt% to about 4 wt% of the cyclodextrin may be used. In another example, the aqueous solution may include from about 1 wt% to about 2 wt% of the cyclodextrin. Cyclodextrins are cup-shaped structures that have a hydrophilic exterior and a hydrophobic core. These compounds have the ability to form complexes with hydrophilic molecules, such as SDS or another chaotropic detergent that is used to remove the transposase 14, 14′. In particular, the long hydrophobic tail of SDS is drawn into the cup of the cyclodextrin, which effectively locks the SDS following transposase 14, 14′ removal and prevents it from denaturing enzymes, including those used in downstream library preparation and/or amplification.

Whether heat or the chaotropic detergent is used, the removal of the transposases 14, 14′ liberates the partially adapted DNA fragments, which include the transferred strands 18, 18′ and the DNA fragments 35, 37 respectively attached thereto. Following this purification step to remove the transposases 14, 14′, the non-transferred strands 20, 20′ are dehybridized and additional sequences (adapters) are added to the 3′ ends of the partially adapted fragments by an extension reaction using exclusion amplification reagents (e.g., the ExAMP reagents available from Illumina Inc.). It is to be understood that in examples where SDS or another chaotropic detergent has been chelated with a cyclodextrin, the extension reaction may take place without having to perform one or more wash cycles to remove the SDS or other chaotropic detergent from the flow cell. The extension reaction involves the addition of nucleotides in a template dependent fashion from the 3′ ends of the DNA fragments 35, 37 using the respective transferred strands 18′, 18 as the template. As such, the DNA fragment 35 is extended along sections 30′, 28′, and 26′ to generate complementary sections 30′C, 28′C, and 26′C attached to the DNA fragment 35; and the DNA fragment 37 is extended along sections 30, 28, and 26 to generate complementary sections 30C, 28C, and 26C attached to the DNA fragment 37.

In short, one example of the methods shown in FIG. 1C and FIG. 1D includes: introducing a DNA sample to a flow cell including depressions 12 separated by interstitial regions 38; first and second primers 36, 36′ immobilized within each of the depressions 12; first transposome complexes 10 immobilized within each of the depressions 12 or on the interstitial regions 38, the first transposome complexes 10 including a first amplification domain 26; and a second transposome complex 10′ immobilized within each of the depressions 12 or on the interstitial regions 38, the second transposome complexes 10′ including a second amplification domain 26′, whereby tagmentation of the DNA sample takes place at some of the first transposome complexes 10 and the second transposome complexes 10′ to generate a plurality of partially adapted DNA fragments; introducing a chaotropic detergent to the flow cell to remove a transposase 14 of the first transposome complexes 10 and of the second transposome complexes 10′; introducing a chelator of the chaotropic detergent to sequester the chaotropic detergent; and performing an extension reaction to add additional adapters to the partially adapted DNA fragments.

The introduction of the chaotropic detergent and the chelator may also be used in methods where the transposome complexes 10, 10′ are attached to another substrate surface, such as a bead (e.g., a magnetic bead). This example method my more generally involve performing a tagmentation reaction with a DNA sample and a surface bound transposome complex 10, 10′, thereby generating a plurality of partially adapted DNA fragments; introducing a chaotropic detergent to remove a transposase 14, 14′ of the transposome complex 10, 10′; introducing a chelator of the chaotropic detergent to sequester the chaotropic detergent; and performing an extension reaction to add additional adapters to the partially adapted DNA fragments. This method may be performed off of the flow cell, and then the beads (with fully adapted DNA fragments bound thereto) may be added to a flow cell including capture sites (for attaching the beads) and primers 36, 36′. The beads can attach to the capture sites and then the fully adapted DNA fragments may be released from the beads and exposed to amplification using the primers on the flow cell surface.

Referring back to FIG. 1C, the sequences resulting from the extension reaction render the partially adapted fragments fully adapted and ready for further amplification and cluster generation.

In one example of cluster generation, the fully adapted fragments shown in FIG. 1D are denatured from one another, and loop over to hybridize to an adjacent, complementary primer 36, 36′, and a polymerase copies the copied templates to form double stranded bridges, which are denatured to form two single stranded strands. These two strands loop over and hybridize to adjacent, complementary primers 36, 36′ and are extended again to form two new double stranded loops. The process is repeated on each template copy by cycles of isothermal denaturation and amplification to create dense clonal clusters. Each cluster of double stranded bridges is denatured. In an example, the reverse strands are removed by a cleaving agent suitable for the cleavage site of the primers 36 or 36′ to which the reverse strands are attached (e.g., specific base cleavage), leaving forward template strands/amplicons. In another example, the forward strands are removed by a cleaving agent suitable for the cleavage site of the primers 36 or 36′ to which the forward strands are attached (e.g., specific base cleavage), leaving reverse template strands/amplicons. Clustering results in the formation of several template strands immobilized in the depressions 12.

Because several of each of the transposome complexes 10, 10′ may be present in a single depression 12 of the flow cell, multiple fully adapted fragments may be present in a single depression 12 after tagmentation and extension. Amplification of multiple fully adapted fragments leads to polyclonality within the depression 12 (i.e., multiple amplicons of each fully adapted fragment), which may render signal resolution of any one type of amplicon more difficult. In some of the examples disclosed herein, the transposome complex 10 and/or 10′ is modified to reduce the tagmentation efficiency of the complex 10 and/or 10′. These modifications can render some of the transposome complexes 10 and/or 10′ within one or more depressions 12 across the flow cell incapable of fully participating in tagmentation, thus increasing the insert size of the DNA fragments that are generated and also decreasing the number of fully adapted fragments within a given depression 12.

Some of the transposome complex 10 and/or 10′ modifications are implemented at the 3′ end of the non-transferred strand 20, 20′.

In one example, the modification to reduce tagmentation efficiency is a fluorophore attached at a 3′ end of the non-transferred strand 20, 20′ of i) some of the first transposome complexes 10, or ii) some of the second transposome complexes 10′, or iii) some of both of the first and second transposome complexes 10, 10′. Examples of suitable fluorophores include those in the ALEXA FLUOR® family of dyes available from Molecular Probes, Inc. (e.g., ALEXA FLUOR® 647, etc.), TET™ Dye Phosphoramidite available from Thermo Fisher Scientific, or the like. The fluorophore may be added to the 3′ end of the non-transferred strand 20, 20′ during its synthesis, and before the transposome complex 10 and/or 10′ is attached to the flow cell surface. This modification partially inactivates the transposome complex 10 or 10′, which leads to fewer tagmentation events or to DNA sample binding but no tagmenting.

Other of the transposome complex 10 and/or 10′ modifications are implemented at the 5′ end of the non-transferred strand 20, 20′.

In one example, the modification to reduce tagmentation efficiency is the exclusion of a phosphate group at a 5′ end of the non-transferred strand 20, 20′ of i) some of the first transposome complexes 10, or ii) some of the second transposome complexes 10′, or iii) some of both of the first and second transposome complexes 10, 10′. The non-transferred strands 20, 20′ may be synthesized without the 5′ phosphate. This modification partially inactivates the transposome complex 10 or 10′, which leads to fewer tagmentation events or to DNA sample binding but no tagmenting.

In another example, the modification to reduce tagmentation efficiency is a fluorophore attached at a 5′ end of the non-transferred strand 20, 20′ of i) some of the first transposome complexes 10, or ii) some of the second transposome complexes 10′, or iii) some of both of the first and second transposome complexes 10, 10′. The fluorophore may be added to the 5′ end of the non-transferred strand 20, 20′ during fabrication of the transposome complex 10, 10′.

In still another example, the modification to reduce tagmentation efficiency involves changing or removing a last base or bases from a 5′ end of the non-transferred strand 20, 20′ of i) some of the first transposome complexes 10, or ii) some of the second transposome complexes 10′, or iii) some of both of the first and second transposome complexes 10, 10′. As examples, the 5′ end bases CAT or CAC may reduce tagmentation efficiency compared to, for example, CAG.

Still other of the transposome complex 10 and/or 10′ modifications are implemented at the 3′ end of the transferred strand 18, 18′. One example of this modification to reduce tagmentation efficiency is a dideoxycytosine, a thymine, or a cytosine attached at the 3′ end of a transferred strand 18, 18′ of i) some of the first transposome complexes 10, or ii) some of the second transposome complexes 10′, or iii) some of both of the first and second transposome complexes 10, 10′. The presence of dideoxycytosine at the 3′ end of the transferred strand 18 and/or 18′ fully inactivates the transposome complex 10 or 10′, which leads to DNA sample binding but not tagmenting. The inclusion of thymine or cytosine at the 3′ end of the transferred strand 18 and/or 18′ leads to reducing the efficiency of tagmentation.

Additionally, any of the modifications that can be made at the 5′ end of the non-transferred strand(s) 20, 20′ can be combined with any of the modifications that can be made at the 3′ end of the non-transferred strand(s) 20, 20′ or any of the modifications that can be made at the 3′ end of the transferred strand(s) 18, 18′. Moreover, any of the any of the modifications that can be made at the 3′ end of the non-transferred strand(s) 20, 20′ can be combined with any of the modifications that can be made at the 3′ end of the transferred strand(s) 18, 18′.

FIG. 13A, FIG. 13B, FIG. 13C, and FIG. 13D illustrate other examples of the transposome complexes 10A, 10B disclosed herein immobilized within a flow cell depression 12.

An example of each of the transposome complexes 10A, 10B is shown, respectively, in more detail in FIG. 14A and FIG. 14B. As depicted, each complex 10A, 10B includes a transposase enzyme 14A, 14B non-covalently bound to a transposon end 16A, 16B. Each transposon end 16A, 16B is a double-stranded nucleic acid strand, one strand 30A, 30B of which is part of the transferred strand 18A, 18B and the other strand 32A, 32B of which is part of the non-transferred strand 20A, 20B. In other words, the transposon end 16A, 16B includes a portion (strand 30A, 30B) of the transferred strand 18A, 18B that is hybridized to a portion (e.g., strand 32A, 32B) of the non-transferred strand 20A, 20B.

In this example of the transposome complex 10A, the transferred strand 18A includes a first amplification domain 26A and a sequencing primer sequence 28A that is attached to one strand 30A of the transposon end 16A. The strand 30A of the transposon end 16A is positioned at the 3′ end of the transferred strand 18A.

Similar to the transferred strand 18A, the transferred strand 18B of the transposome complex 10B includes a second amplification domain 26B and a sequencing primer sequence 28B that is attached to one strand 30B of the transposon end 16B. The strand 30B of the transposon end 16B is positioned at the 3′ end of the transferred strand 18B.

Similar to the first and second amplification domains 26, 26′ of the transposome complexes 10, 10′, the first and second amplification domains 26A, 26B of the transposome complexes 10A, 10B have different sequences from each other, but have the same sequence, respectively, as first and second primers 36A, 36B attached to the polymer hydrogel 24, 24′ (which, as described below, may be biotinylated). The first amplification domain 26A and the primer 36A together with the second amplification domain 26B and the primer 36B enable the amplification of the DNA sample fragments generated during tagmentation. Any of the sequences set forth herein for the first amplification domain 26/primer 36 and for the second amplification domain 26′/primer 36′ may be used, respectively, for the first amplification domain 26A/primer 36A and for the second amplification domain 26B/primer 36B.

Similar to the sequencing primer sequences 28, 28′, the sequencing primer sequences 28A, 28B have different sequences from each other that respectively bind to sequencing primers introduced into the flow cell after tagmentation and amplification.

The transposon ends 16A, 16B of each transposome complex 10A, 10B include the strands 30A, 30B respectively hybridized to the strands 32A, 32B. As such, the strands 30A and 32A are complementary and the strands 30B, 32B are complementary. The double stranded transposon ends 16A, 16B are respectively capable of complexing with the transposases 14A, 14B. As examples, the strands 30A, 32A and 30B, 32B of the transposon ends 16A, 16B may be the related but non-identical 19-base pair (bp) outer end (e.g., strands 30, 30′) and inner end (e.g., strands 32, 32′) sequences that serve as the substrate for the activity of the Tn5 transposase, or the mosaic ends recognized by a wild-type or mutant Tn5 transposase, or the R1 end (e.g., strands 30, 30′) and the R2 end (strands 32, 32′) recognized by the MuA transposase.

The transposome complexes 10A, 10B are configured for asymmetric attachment to the flow cell surface (as shown in FIG. 13A). As such, one of the complexes, e.g., complex 10A, includes 3′ end group 22A for attachment to the flow cell surface, and the other of the complexes, e.g., complex 10B, includes a 5′ end group 22B for attachment to the flow cell surface. Thus, as depicted in FIG. 13A, FIG. 14A, and FIG. 14B, the non-transferred strand 20A of the complex 10A includes the 3′ end group 22A and the transferred strand 18B of the complex 10B includes the 5′ end group 22B. The 3′ end group 22A and the 5′ end group 22B may be any functional group that is capable of covalently or non-covalently attaching, directly or indirectly, to surface functional groups of the polymeric hydrogel 24, 24′, and thus will depend upon the surface functional groups of the polymer hydrogel 24, 24′. In one example, the polymeric hydrogel 24 includes azide or tetrazine surface groups, and the 3′ end group 22A and the 5′ end group 22B each include a terminal alkyne (e.g., hexynyl) or an internal alkyne, where the alkyne is part of a cyclic compound (e.g., bicyclo[6.1.0]nonyne (BCN)). In another example, the biotinylated polymeric hydrogel 24′ is present in the depressions 12 of the flow cell, and each of the 3′ end group 22A and the 5′ end group 22B is biotin. In these examples, additional streptavidin or avidin is added to indirectly attach the biotin groups to one another.

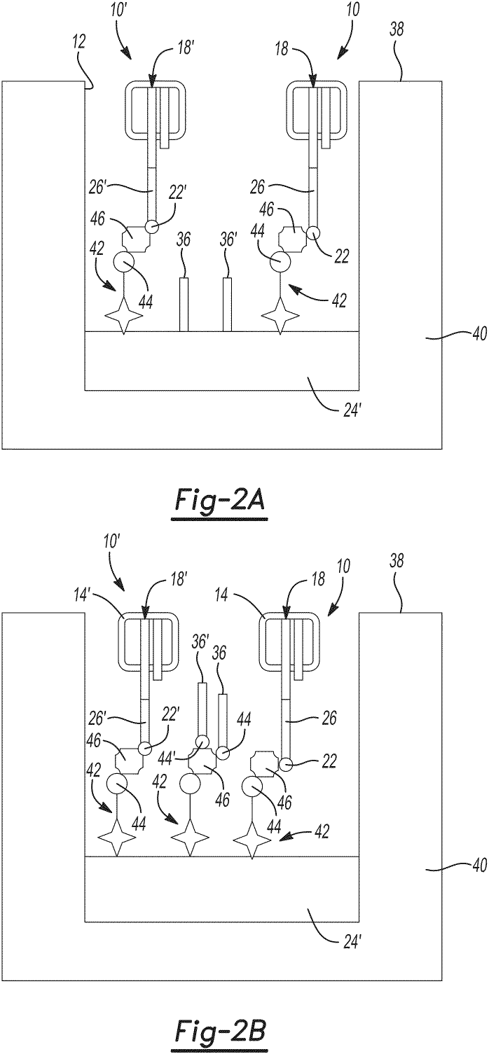

Referring back to FIG. 13A, the transposome complexes 10A, 10B may be introduced to the flow cell, which includes the depressions 12 separated by the interstitial regions 38 and an example of the polymeric hydrogel 24 or 24′ (the latter of which is described in detail in reference to FIG. 2A and FIG. 2B) in the depressions 12.

The transposome complexes 10A, 10B may also be included in a carrier liquid in a concentration ranging from about 0.1 µM to about 1 µM. The carrier liquid of this example of the transposome complex fluid may be water. When the polymeric hydrogel 24 is used, a buffer and/or salt may be added to the carrier liquid for grafting the transposome complexes 10A, 10B to suitable functional groups of the polymeric hydrogel 24. The buffer has a pH ranging from 5 to 12. Any of the neutral buffers and/or salts set forth herein may be added to this example of the transposome complex fluid.

For grafting, the transposome complex fluid is introduced into the flow cell. The transposome complex fluid may be introduced using flow through deposition. Grafting may be performed at a temperature ranging from about 35° C. to about 45° C. for a time ranging from about 30 minutes to about 120 minutes. During grafting, the transposome complexes 10A, 10B attach to at least some of the azide or tetrazine groups of the polymeric hydrogel 24 and have no affinity for the interstitial regions 38 or edge portions of the flow cell.

When the biotinylated polymeric hydrogel 24′ is used, the attachment of the transposome complexes 10A, 10B to the flow cell surface may take place using any of the examples described herein in more detail in reference to FIG. 2A through FIG. 5. Briefly, it is to be understood that avidin or streptavidin may be used to attach the 3′ and 5′ ends 22A, 22B (which in this example are biotinylated) with the biotinylated polymeric hydrogel 24′ as described herein in the section “Flow Cells”. As examples, the avidin or streptavidin may be pre-attached to the 3′ and 5′ biotinylated ends 22A, 22B before the complexes 10A, 10B are introduced into the flow cell and allowed to incubate; or the avidin or streptavidin may be introduced into the flow cell with the 3′ and 5′ biotinylated transposome complexes 10A, 10B and allowed to incubate; or the avidin or streptavidin may be introduced into the flow cell and allowed to incubate with the polymeric hydrogel 24, and then the 3′ and 5′ biotinylated transposome complexes 10A, 10B may be added and allowed to incubate; or the avidin or streptavidin and biotin are pre-attached to one another and then introduced into the flow cell where the biotin attaches to the surface of the polymeric hydrogel 24 through some of the R^{A} groups (directly or through a linker), and then the biotinylated transposome complexes 10A, 10B may be introduced into the flow cell containing the streptavidin-biotin bound pair as part of the polymeric hydrogel 24′. As mentioned, more details of each of these methods are set forth herein in reference to FIG. 2 through FIG. 5.

The flow cell shown in FIG. 13A also includes the primers 36A, 36B. Any of the primers 36, 36′ and suitable attachment mechanisms of the primers 36, 36′ to the polymeric hydrogel 24 or 24′ described herein may be used for the primers 36A, 36B.

In this example, the plurality of first transposome complexes (e.g., the transposome complexes 10A, which may be referred to herein as the 3′ attached transposome complexes 10A) and the plurality of second transposome complexes (e.g., transposome complexes 10B, which may be referred to herein as the 5′ attached transposome complexes 10B) are present in the flow cell at a ratio ranging from greater than 1:1 to 5:1. In another example, the 3′ biotinylated transposome complexes 10A and the 5′ biotinylated transposome complexes 10B are introduced into the flow cell at a ratio of 4:1. As will be described in more detail in reference to FIG. 13C and FIG. 13D, the asymmetrically attached complexes 10A, 10B introduced into, and thus present on the flow cell surface at a ratio within the given range, may improve the pass filter percentages.

FIG. 13B illustrates the depression 12 after a DNA sample has been introduced. The DNA sample may be introduced with a tagmentation buffer, which may include water, an optional co-solvent (e.g., dimethylformamide), a metal co-factor for the transposase (e.g., magnesium acetate), and a buffer salt (e.g., Tris acetate salt, pH 7.6). In an example, the optional co-solvent may be present in an amount up to about 11%, the metal co-factor may be present in a concentration ranging from about 3 mM to about 5.5 mM, and the buffer salt may be present in a concentration ranging from about 7 mM to about 12 mM.

When the DNA sample (shown fragmented at reference numeral 34) is introduced into the flow cell including the 3′ and 5′ attached transposome complexes 10A, 10B, the DNA sample is fragmented and the 5′ ends of both strands 35, 37 of the duplex fragments 34 are ligated to respective 3′ ends of the transferred strands 18A, 18B of the transposome complexes 10A, 10B. Fragmentation and ligation may take place at a temperature at or above 30° C. In one example, the temperature may range from 30° C. to about 55° C. In another example, the temperature may range from 35° C. to about 45° C. The 3′ ends of the duplex fragments 34 are not ligated to the 5′ ends of the non-transferred strands 20A, 20B. As such, a gap 39A exists between the 3′ end of the DNA fragment strand 37 and the 5′ end of the non-transferred strand 20A, and a gap 39B exists between the 3′ end of the DNA fragment strand 35 and the 5′ end of the non-transferred strand 20B. In one example, each gap 39A, 39B is nine (9) base pairs long.

The transposases 14A, 14B are then removed from the complexes 10A, 10B, which are now attached to the fragments 35, 37 via the transferred strands 18A, 18B. Transposase removal 14A, 14B may be accomplished by heating the flow cell. In this example, a washing solution is introduced into the flow cell; the flow cell containing the washing solution is heated to about 60° C.; and then the extension amplification mix is introduced into the flow cell. Prior to the introduction of the extension amplification mix, the temperature may be lowered to about 38° C.

The extension mix is then added to form the fully extended fragment with adapters at both ends. Two examples of the extension reaction are illustrated in FIG. 13C and FIG. 13D. In both examples, the extension of the fragments 35, 37 involves the addition of nucleotides in a template dependent fashion from the 3′ ends of the DNA fragments 35, 37. FIG. 13C illustrates extension when at least some of the strands 18A, 20A and 18B, 20B remain hybridized after transposase removal 14A, 14B. In this example, the extension reaction may displace the non-transferred strands 20A, 20B and allow the transferred strands 18A, 18B to be copied, thus forming fully extended (fully adapted) fragments (similar to the example shown in FIG. 1D). FIG. 13D illustrates extension when at least some of the strands 18A, 20A and 18B, 20B dehybridize during transposase removal 14A, 14B. Because the non-transferred strands 20A, 20B are relatively short, their melting temperature may be low (e.g., from about 40° C. to about 50° C.); as such, the transposase removal temperature may be sufficient to dehybridize at least some of the transposon ends. In contrast, the longer fragments 35, 37 may remain hybridized. It is to be understood that the longer fragments 35, 37 may dehybridize at regions (e.g., AT rich regions) that have a lower melting temperature, but the overall insert size includes regions with higher melting temperature that do not dehybridize. These regions keep the longer double stranded fragments 35, 37 from falling apart. The insert size may be shifted if the transposase removal temperature is increased above 60° C., which can lead to dehybridization of some of the double stranded fragments 35, 37.

In another example, transposase removal 14A, 14B is accomplished using sodium dodecyl sulfate (SDS) or proteinase, followed by the introduction of the wash solution, dehybridization of the non-transferred strands 20B, and the extension amplification reaction.

In any of the examples disclosed herein, it is to be understood that random tagmentation can take place during the tagmentation process. By “random tagmentation,” it is meant that the 5′ ends of both strands of a duplex fragment are ligated to respective 3′ ends of the same type of transposome complex 10A or 10B (or 10 or 10′). An example of random tagmentation is shown in FIG. 13B, where each of the strands 35′, 37′ is respectively ligated to a 3′ end of a transferred strand 18A (of complex 10A). After the extension amplification reaction, these fully adapted fragments have the same amplification domain 26A at both ends (e.g., in the original transferred strand 18A and in the complementary section). While not shown, it is to be understood that both strands of a duplex fragment may alternatively be ligated to respective 3′ ends of the transferred strand 18B (of complex 10B). After the extension reaction, these fully adapted fragments would have the same amplification domain 26B (e.g., in the original transferred strand 18B and in the complementary section), at both ends.

Fully adapted fragments with the same amplification domain 26A or 26B at both ends can reduce the percentage of reads that are able to pass filter. However, the asymmetrical attachment of the transposome complexes 10A, 10B and the ratio of 3′ attached transposome complexes 10A to 5′ attached transposome complexes 10B generates a higher yield of fully adapted fragments with different amplification domains 26A, 26B at the ends.

### Flow Cells

The transposome complexes 10, 10′, 10A, 10B, 10C, 10D disclosed herein, whether modified or non-modified, are immobilized on a flow cell surface. As shown in FIG. 1A through FIG. 1D, in one example, the transposome complexes 10, 10′ are attached to the polymeric hydrogel 24 within the depression 12 of the flow cell. As shown in FIG. 13A through FIG. 13D, in another example, the transposome complexes 10A, 10B are asymmetrically attached to the polymeric hydrogel 24, 24′ within the depression 12 of the flow cell. While a single depression 12 is shown in each of these examples, it is to be understood that a plurality of depressions 12 may be formed across the substrate surface in an array as described herein.

Each example of the flow cell disclosed herein includes a substrate 40 having depressions 12 separated by interstitial regions 38 (see FIG. 1A through FIG. 1D), and a polymeric hydrogel 24 within the depressions 12. This type of substrate may be referred to as a patterned substrate, and the flow cell may include two patterned substrates bonded together, or may include a single patterned substrate bonded to a lid.

A flow channel is defined between the substrates or the substrate and the lid. The depth of the flow channel can be as small as a monolayer thick when microcontact, aerosol, or inkjet printing is used to deposit a material to bond the substrates or the substrate and the lid. In other examples, the depth of the flow channel can be about 1 µm, about 10 µm, about 50 µm, about 100 µm, or more. In an example, the depth may range from about 10 µm to about 100 µm. In another example, the depth may range from about 10 µm to about 30 µm. In still another example, the depth is about 5 µm or less. It is to be understood that the depth of the flow channel may be greater than, less than or between the values specified above.

Each flow channel is in fluid communication with an inlet and an outlet (not shown). The inlet and outlet may be positioned at opposed ends of the flow cell. The inlets and outlets of the respective flow channels may alternatively be positioned anywhere along the length and width of the flow channel that enables desirable fluid flow.

The inlet allows fluids to be introduced into the flow channel, and the outlet allows fluid to be extracted from the flow channel. Each of the inlets and outlets is fluidly connected to a fluidic control system (including, e.g., reservoirs, pumps, valves, waste containers, and the like) which controls fluid introduction and expulsion.

The substrate 40 may be a single layer base support having the depressions 12 defined therein, or a multi-layer structure including a base support with another layer positioned thereon and having the depressions 12 defined therein.

Examples of suitable single layer base supports include epoxy siloxane, glass, modified or functionalized glass, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon (polyamides), ceramics/ceramic oxides, silica, fused silica, or silica-based materials, aluminum silicate, silicon and modified silicon (e.g., boron doped p+ silicon), silicon nitride (Si₃N₄), silicon oxide (SiO₂), tantalum pentoxide (Ta₂O₅) or other tantalum oxide(s) (TaOₓ), hafnium oxide (HfO₂), carbon, metals, inorganic glasses, or the like.

Examples of the multi-layered structure include any example of the base support and at least one other layer on the base support.

The other layer may be an inorganic oxide, such as tantalum oxide (e.g., Ta₂O₅), aluminum oxide (e.g., Al₂O₃), silicon oxide (e.g., SiO₂), hafnium oxide (e.g., HfO₂), etc. Inorganic oxides could be selectively applied, e.g., via vapor deposition, aerosol printing, or inkjet printing, to form the depressions 12 and interstitial regions 38.

The other layer may alternatively be a polymeric resin that can be applied to the base support and then patterned. Some examples of suitable polymeric resins include a polyhedral oligomeric silsesquioxane based resin, an epoxy resin not based on a polyhedral oligomeric silsesquioxane, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof. Suitable deposition techniques include chemical vapor deposition, dip coating, dunk coating, spin coating, spray coating, puddle dispensing, ultrasonic spray coating, doctor blade coating, aerosol printing, screen printing, microcontact printing, etc. Suitable patterning techniques include photolithography, nanoimprint lithography (NIL), stamping techniques, embossing techniques, molding techniques, microetching techniques, etc.

In an example, the substrate 40 may be a circular sheet, a panel, a wafer, a die etc. having a diameter ranging from about 2 mm to about 300 mm, e.g., from about 200 mm to about 300 mm, or may be a rectangular sheet, panel, wafer, die etc. having its largest dimension up to about 10 feet (~ 3 meters). While example dimensions have been provided, it is to be understood that a substrate 40 with any suitable dimensions may be used.

The depressions 12 may be formed in the substrate 40 using any suitable patterning techniques, such as photolithography, nanoimprint lithography (NIL), stamping techniques, embossing techniques, molding techniques, microetching techniques, etc.

Many different layouts of the depressions 12 and interstitial regions 38 may be envisaged, including regular, repeating, and non-regular patterns. In an example, the plurality depressions 12 and interstitial regions 38 are disposed to create a hexagonal grid for close packing and improved density. Other layouts may include, for example, rectangular layouts, triangular layouts, and so forth. In some examples, the layout or pattern can be an x-y format in rows and columns. In some other examples, the layout or pattern can be a repeating arrangement of the depressions 12 and the interstitial regions 38. In still other examples, the layout or pattern can be a random arrangement of the plurality of depressions 12 and the interstitial regions 38.

The layout or pattern may be characterized with respect to the density (number) of the plurality depressions 12 within a defined area. For example, the depressions 12 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high density array may be characterized as having the plurality of depressions 12 separated by less than about 100 nm, a medium density array may be characterized as having the plurality of depressions 12 separated by about 400 nm to about 1 µm, and a low density array may be characterized as having the plurality of depressions 12 separated by greater than about 1 µm.

The layout or pattern of the plurality of depressions 12 may also or alternatively be characterized in terms of the average pitch, or the spacing from the center of one depression 12 to the center of an adjacent depression 12 (center-to-center spacing) or from the right edge of one depressions 12 to the left edge of an adjacent depression 12 (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.15 µm, about 0.5 µm, about 1 µm, about 5 µm, about 10 µm, about 100 µm, or more or less. The average pitch for a particular pattern of can be between one of the lower values and one of the upper values selected from the ranges above. In an example, the depressions 12 have a pitch (center-to-center spacing) of about 1.5 µm. While example average pitch values have been provided, it is to be understood that other average pitch values may be used.

The size of each depression 12 may be characterized by its volume, opening area, depth, and/or diameter (when the depression 12 is circular) and/or length and width. For example, the volume can range from about 1×10⁻³ µm³ to about 100 µm³, e.g., about 1×10⁻² µm³, about 0.1 µm³, about 1 µm³, about 10 µm³, or more, or less. For another example, the opening area can range from about 1×10⁻³ µm² to about 100 µm², e.g., about 1×10⁻² µm², about 0.1 µm², about 1 µm², at least about 10 µm², or more, or less. For still another example, the depth can range from about 0.1 µm to about 100 µm, e.g., about 0.5 µm, about 1 µm, about 10 µm, or more, or less. For another example, the depth can range from about 0.1 µm to about 100 µm, e.g., about 0.5 µm, about 1 µm, about 10 µm, or more, or less. For yet another example, the diameter or each of the length and width can range from about 0.1 µm to about 100 µm, e.g., about 0.5 µm, about 1 µm, about 10 µm, or more, or less.

The substrate 40 may include edge regions that define interstitial like regions that extend the length of the flow channels and separate one flow channel from an adjacent flow channel. The edge regions provide bonding regions where two substrates 40 can be attached to one another or where one substrate 40 can be attached to the lid.

The depressions 12 provide a designated area for the polymeric hydrogel 24. In the examples disclosed herein, the polymeric hydrogel 24 includes an acrylamide copolymer. In this example, the acrylamide copolymer has a structure:

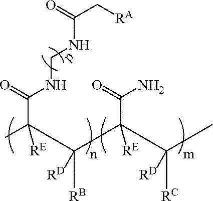

wherein:
R^{A} is an azide or a tetrazine or any other functional group that can attach to an alkyne;
R^{B} is H or optionally substituted alkyl;
R^{C}, R^{D}, and R^{E} are each independently selected from the group consisting of H and optionally substituted alkyl;
each of the —(CH₂)ₚ— can be optionally substituted;
p is an integer in the range of 1 to 50;
n is an integer in the range of 1 to 50,000; and
m is an integer in the range of 1 to 100,000.

One specific example of the acrylamide copolymer represented by structure (I) is poly(N-(5-azidoacetamidylpentyl)acrylamide-co-acrylamide, PAZAM.

One of ordinary skill in the art will recognize that the arrangement of the recurring “n” and “m” features in structure (I) are representative, and the monomeric subunits may be present in any order in the polymer structure (e.g., random, block, patterned, or a combination thereof).

The molecular weight of the acrylamide copolymer may range from about 5 kDa to about 1500 kDa or from about 10 kDa to about 1000 kDa, or may be, in a specific example, about 312 kDa.

In some examples, the acrylamide copolymer is a linear polymer. In some other examples, the acrylamide copolymer is a lightly cross-linked polymer.

In some examples, the gel material may be a variation of structure (I). In one example, the acrylamide unit may be replaced with N,N-dimethylacrylamide

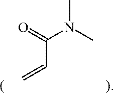

In another example, the acrylamide unit in structure (I) may bereplaced with,

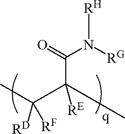

where R^{D}, R^{E}, and R^{F} are each H or a C1-C6 alkyl, and R^{G} and R^{H} are each a C1-C6 alkyl (instead of H as is the case with the acrylamide). In this example, q may be an integer in the range of 1 to 100,000. In another example, the N,N-dimethylacrylamide may be used in addition to the acrylamide unit. In this example, structure (I) may include

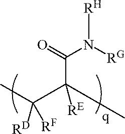

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As another example of the polymeric hydrogel, the recurring “n” feature in structure (I) may be replaced with a monomer including a heterocyclic azido group having structure (II):

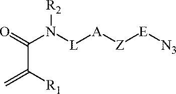

wherein R¹ is H or a C1-C6 alkyl; R₂ is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl. As still another example, the gel material may include a recurring unit of each of structure (III) and (IV):

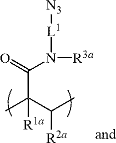

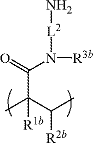

wherein each of R^{1a}, R^{2a}, R^{1b} and R^{2b} is independently selected from hydrogen, an optionally substituted alkyl or optionally substituted phenyl; each of R^{3a} and R^{3b} is independently selected from hydrogen, an optionally substituted alkyl, an optionally substituted phenyl, or an optionally substituted C7-C14 aralkyl; and each L¹ and L² is independently selected from an optionally substituted alkylene linker or an optionally substituted heteroalkylene linker.

In some examples, the polymeric hydrogel 24 is biotinylated. In these examples, biotin is attached to the surface of the polymeric hydrogel 24 through some of the R^{A} groups (i.e., the azide, tetrazine, or other functional group that can attach to an alkyne). The biotin is attached to a linker, such as bicyclo[6.1.0]nonyne (BCN), which can covalently attach to some of the R^{A} groups. The combination of the biotin and the linker is one example of a biotin-containing linker, which is shown at reference numeral 42 at least in FIG. 2A and FIG. 2B.

In other examples, streptavidin and biotin are attached to one another, and the biotin is attached to the surface of the polymeric hydrogel 24 through some of the R^{A} groups (i.e., the azide, tetrazine, or other functional group that can attach to an alkyne). The biotin may be attached to a linker, such as bicyclo[6.1.0]nonyne (BCN), which can covalently attach to some of the R^{A} groups. In this example, the biotin-containing linker includes the linker, the biotin, and the streptavidin.

In still other examples, the bicyclo[6.1.0]nonyne (BCN) linkers can be attached to some of the R^{A} groups of the polymeric hydrogel 24, and the biotin is not attached. In these examples, biotin can added after the polymeric hydrogel 24 is applied to the depressions 12.

To introduce the polymeric hydrogel 24 into the depressions 12, a mixture of the polymeric hydrogel 24 may be generated and then applied to the substrate 40. In one example, the polymeric hydrogel 24 may be present in a mixture (e.g., with water or with ethanol and water). The mixture may then be applied to the substrate surface using spin coating, or dipping or dip coating, or flow of the material under positive or negative pressure, or another suitable technique. These types of techniques blanketly deposit the polymeric hydrogel 24 in the depressions 12 and on the interstitial regions 38. Other selective deposition techniques (e.g., involving a mask, controlled printing techniques, etc.) may be used to specifically deposit the polymeric hydrogel 24 in the depressions 12 and not on the interstitial regions 38.

In some examples, the substrate 40 may be activated, and then the mixture (including the polymeric hydrogel 24) may be applied thereto. In one example, a silane or silane derivative (e.g., norbornene silane) may be deposited on the surface of the substrate 40 using vapor deposition, spin coating, or other deposition methods. In another example, the substrate 40 may be exposed to plasma ashing to generate surface-activating agent(s) (e.g., —OH groups) that can adhere to the polymeric hydrogel 24.

The applied mixture may be exposed to a curing process to cure the polymeric hydrogel 24. In an example, curing may take place at a temperature ranging from room temperature (e.g., about 25° C.) to about 95° C. for a time ranging from about 1 millisecond to about several days.

Polishing may then be performed in order to remove the polymeric hydrogel 24 from the interstitial regions 38, while leaving the polymeric hydrogel 24 on the surface in the depressions 12 at least substantially intact. The polishing process may be performed with a chemical slurry (including, e.g., an abrasive, a buffer, a chelating agent, a surfactant, and/or a dispersant) which can remove the polymeric hydrogel 24 from the interstitial regions 38 without deleteriously affecting the underlying substrate at those regions 38. Alternatively, polishing may be performed with a solution that does not include the abrasive particles.

The chemical slurry may be used in a chemical mechanical polishing system to polish the surface of the interstitial regions 38. The polishing head(s)/pad(s) or other polishing tool(s) is/are capable of polishing the polymeric hydrogel 24 that may be present over the interstitial regions 38 while leaving the polymeric hydrogel 24 in the depressions 12 at least substantially intact. As an example, the polishing head may be a Strasbaugh ViPRR II polishing head.

Cleaning and drying processes may be performed after polishing. The cleaning process may utilize a water bath and sonication. The water bath may be maintained at a relatively low temperature ranging from about 22° C. to about 30° C. The drying process may involve spin drying, or drying via another suitable technique.

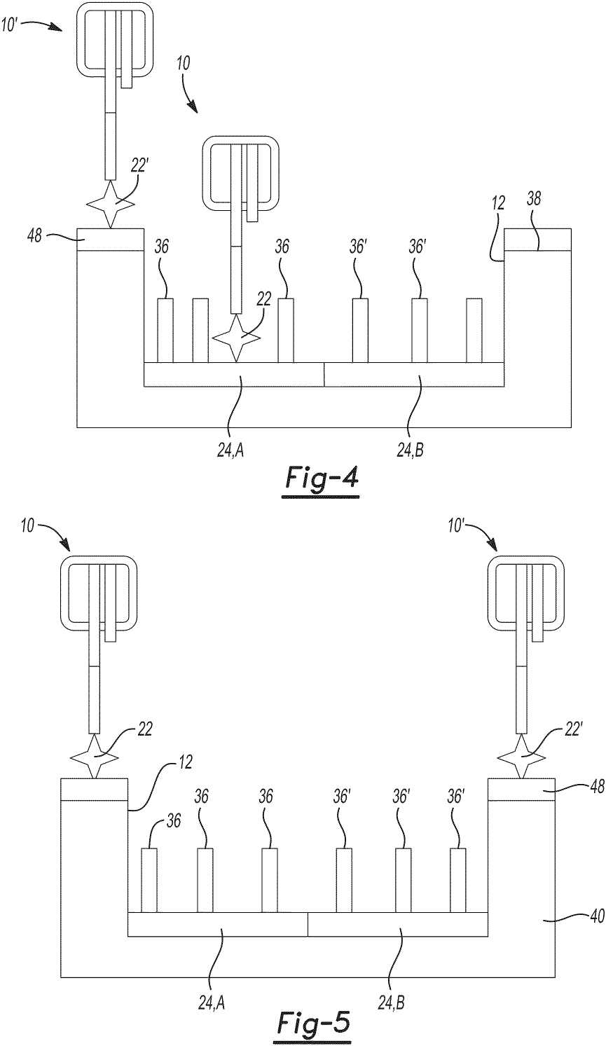

Each example of the flow cell also includes the primers 36, 36′ and the transposome complexes 10, 10′. The primers 36, 36′ include any two of the primer sequences set forth herein for the first and second amplification domains 26, 26′. The 5′ terminal end of the primers 36, 36′ will vary depending upon the chemistry of the polymeric hydrogel 24. Different examples of how the primers 36, 36′ and the transposome complexes 10, 10′ can be attached within the flow cell will be described in reference to FIG. 1A, FIG. 2A, FIG. 2B, FIG. 3A, FIG. 3B, FIG. 4, and FIG. 5.

As shown in FIG. 1A, both of the primers 36, 36′ and both of the transposome complexes 10, 10′ are attached to the polymeric hydrogel 24 in the depression 12. In one example, the 5′ ends of the primers 36, 36′ and of the transposome complexes 10, 10′ include functional groups that can covalently attach to the azide and/or tetrazine functional groups of the polymeric hydrogel 24. As examples, the 5′ end functional groups 22, 22′ may be a terminal alkyne (e.g., hexynyl) or an internal alkyne, where the alkyne is part of a cyclic compound (e.g., bicyclo[6.1.0]nonyne (BCN)).

The primers 36, 36′ may be included in a carrier liquid in a concentration ranging from about 0.5 µM to about 100 µM. In one example, the primer concentration ranges from about 5 µM to about 25 µM.

The carrier liquid of the primer fluid may be water. A buffer and/or salt may be added to the carrier liquid for grafting the primers 36, 36′ to suitable functional groups of the polymeric hydrogel 28. The buffer has a pH ranging from 5 to 12, and the buffer used will depend upon the alkyne at the 5′ end of the primers 36, 36′. A neutral buffer and/or salt may be added to the primer fluid for grafting BCN terminated primers, while an alkaline buffer may be added to the primer fluid for copper-assisted grafting methods (e.g., the click reaction). Any of the primer fluids used in copper-assisted grafting methods may also include a copper catalyst. Example of neutral buffers include Tris(hydroxymethyl) aminomethane (Tris or TRIS) buffers, such as Tris-HCI or Tris-EDTA, or a carbonate buffer (e.g., 0.25 M to 1 M). Sodium sulfate (e.g., 1 M to 2 M) is a suitable salt that may be used. Examples of alkaline buffers include Tris(hydroxymethyl) aminomethane (CHES), 3-(Cyclohexylamino)-1-propanesulphonic acid (CAPS), and alkaline buffer solution (from Sigma-Aldrich).

For grafting, the primer fluid is introduced into the flow cell. The primer fluid may be introduced using flow through deposition. Grafting may be performed at a temperature ranging from about 55° C. to about 65° C. for a time ranging from about 20 minutes to about 60 minutes. In one example, grafting is performed at 60° C. for about 30 minutes or 60 minutes. It is to be understood that a lower temperature and a longer time or a higher temperature and a shorter time may also be used. Some primer grafting techniques, such as those involving BCN grafting to tetrazine units, may be performed at room temperature (e.g., 18° C. to about 25° C.). During grafting, the primers 36, 36′ attach to at least some of the azide or tetrazine groups of the polymeric hydrogel 24 and have no affinity for the interstitial regions 38 or edge portions of the flow cell.

The transposome complexes 10, 10′ may also be included in a carrier liquid in a concentration ranging from about 0.1 µM to about 1 µM.

The carrier liquid of the transposome complex fluid may be water. A buffer and/or salt may be added to the carrier liquid for grafting the transposome complexes 10, 10′ to suitable functional groups of the polymeric hydrogel 24. The buffer has a pH ranging from 5 to 12. Any of the neutral buffers and/or salts set forth herein may be added to the transposome complex fluid.

For grafting, the transposome complex fluid is introduced into the flow cell. The transposome complex fluid may be introduced using flow through deposition. Grafting may be performed at a temperature ranging from about 35° C. to about 45° C. for a time ranging from about 30 minutes to about 120 minutes. In one example, grafting is performed at 37° C. for about 90 minutes or 120 minutes. During grafting, the transposome complexes 10, 10′ attach to at least some of the azide or tetrazine groups of the polymeric hydrogel 24 and have no affinity for the interstitial regions 38 or edge portions of the flow cell.

As shown in FIG. 1A, the separate transposome complexes 10, 10′ form dimers in solution, and these dimers attach to the polymeric hydrogel 24. It is to be understood that FIG. 1B through FIG. 1C illustrate one transposome complex 10 from one dimer and one transposome complex 10′ from another dimer for simplicity and ease of illustration.

Transposome complex 10, 10′ grafting may take place during flow cell manufacturing. Alternatively, the transposome complex fluid may be contained in a reagent cartridge, and transposome complex 10, 10′ grafting may take place on the sequencing instrument prior to sample preparation.

Similar to FIG. 1A, FIG. 2A and FIG. 2B illustrate the both of the primers 36, 36′ and both of the transposome complexes 10, 10′ attached to the polymeric hydrogel 24 in the depression 12. The attachment mechanisms in FIG. 2A and FIG. 2B utilize the non-covalent interaction between biotin and streptavidin.

In the examples shown in FIG. 2A and FIG. 2B, the polymeric hydrogel 24 is biotinylated. The biotinylated polymeric hydrogel 24′ contains biotin-containing linkers 42 grafted to the surface of the polymeric hydrogel 24. The biotinylated polymer hydrogel 24′ may be prepared by grafting bicyclononyne-biotin (BCN-biotin) to some of terminal azide or tetrazine groups of the polymeric hydrogel 24. The biotin-containing linkers 42 may be grafted to the polymeric hydrogel 24 prior to the polymeric hydrogel 24 being added to the flow cell surface, or after the polymeric hydrogel 24 has been added to the flow cell surface.

In one example method, the polymeric hydrogel 24 is applied to the depressions 12 as described in reference to FIG. 1A, and then the biotin-containing linkers 42 grafted to the surface of the polymeric hydrogel 24. In this example, the biotin-containing linkers 42 may be added to a carrier fluid (e.g., water including a neutral buffer and/or salt as described herein), and the fluid may be introduced to the flow cell and allowed to incubate. Grafting may be performed at a temperature ranging from about 50° C. to about 70° C. for a time ranging from about 30 minutes to about 120 minutes. In one example, biotin-containing linker 42 grafting is performed at 60° C. for about 120 minutes. During grafting, the biotin-containing linkers 42 attach to at least some of the azide or tetrazine groups of the polymeric hydrogel 24 and have no affinity for the interstitial regions 38 or edge portions of the flow cell. Once grafted, the biotin 44 of the biotin-containing linkers 42 is at the surface, and thus is available for interaction with subsequently introduced streptavidin 46.

In the example shown in FIG. 2A, the primers 36, 36′ are grafted to some other of the terminal azide or tetrazine groups of the biotinylated polymer hydrogel 24′. Primer grafting may be performed as described in reference to FIG. 1A.

It is to be understood that the transposome complexes 10, 10′ used in the example shown in FIG. 2A include biotin as the 5′ end functional groups 22, 22′. Thus, the transferred strands 18, 18′ of these example transposome complexes 10, 10′ include a 5′ biotinylated end.

In one example, these biotinylated transposome complexes 10, 10′ are introduced to the flow cell, which includes the depressions 12 separated by the interstitial regions 38 and the biotinylated polymeric hydrogel 24′ in the depressions 12. This example method also includes introducing streptavidin 46 to the flow cell, and incubating the transposome complexes 10, 10′ and the streptavidin 46 in the flow cell, whereby the streptavidin 46 respectively attaches the 5′ biotinylated end 22, 22′ of at least some of the transposome complexes 10, 10′ to the biotinylated polymeric hydrogel 24′. Incubation of the transposome complexes 10, 10′ and the streptavidin 46 in the flow cell may take place at room temperature (e.g., from about 18° C. to about 25° C.) for a time ranging from about 15 minutes to about 60 minutes. In one example, this incubation takes place for about 30 minutes at about 22° C.

In this example, the transposome complexes 10, 10′ and the streptavidin 46 may be added at a weight ratio ranging from about 1:1 to about 1:2 so that an ample amount of streptavidin is present to graft the transposome complexes 10, 10′ to the biotin-containing linkers 42.

As depicted in FIG. 2A, the streptavidin 46 interacts with the biotin at the surface of the biotinylated polymeric hydrogel 24′ and with the 5′ end biotin functional groups 22, 22′.

The example shown in FIG. 2B is similar to the example shown in FIG. 2A, except that the primers 36, 36′ are also biotinylated. Instead of the 5′ end of the primers 36, 36′ containing an alkyne, the 5′ end contains biotin 44′. These primers 36, 36′ are referred to as biotinylated primers.

In this example, biotinylated transposome complexes (i.e., complexes 10, 10′ with 5′ end biotin functional groups 22, 22′) and the biotinylated primers (i.e., primers 36, 36′ with 5′ end biotin) respectively attach to biotin-containing linkers 42 of the biotinylated polymeric hydrogel 24′. In this example, streptavidin 46 is introduced into the flow cell with the biotinylated transposome complexes in order to achieve the desired biotin 44-streptavidin 46-biotin 22, 22′ interaction, and streptavidin 46 is introduced into the flow cell with the biotinylated primers in order to achieve the desired biotin 44-streptavidin 46-biotin 44′ interaction. Thus, in this example, the first and second biotinylated primers (36, 36′ with 44′) are immobilized to the biotinylated polymeric hydrogel 24′ through streptavidin 46; the first biotinylated transposomes (10 with biotin 5′ end functional groups 22) are immobilized to the biotinylated polymeric hydrogel 24′ through streptavidin 46; and the second biotinylated transposome complexes (10′ with biotin 5′ end functional groups 22′) are immobilized to the biotinylated polymeric hydrogel 24′ through streptavidin 46.

Because the biotin-streptavidin interactions are reversible, the example of the flow cell shown in FIG. 2B may be reusable. For example, after DNA sample tagmentation, fragment amplification, clustering, and sequencing, a biotin streptavidin cleavage composition may be introduced into the flow cell. Examples of the biotin streptavidin cleavage composition include about 95% formamide and about 10 mM ethylenediaminetetraacetic acid (EDTA), or from about 10% by volume to about 50% by volume of a formamide reagent and a balance of a salt buffer. At suitable reaction temperatures, these cleavage compositions disrupt the biotin-streptavidin interactions, thus releasing whatever is attached (e.g., DNA fragments, nascent strands, etc.) to the biotin 22, 22′, 44′. Alternatively a hot wash with biotin and/or desthiobiotin causes the newly added biotin and/or desthiobiotin to compete with the already bound biotin.

Thus, the example shown in FIG. 2B is a reusable flow cell, comprising a substrate 40 including depressions 12 separated by interstitial regions 38; a biotinylated polymeric hydrogel 24′ in the depressions 12; first and second biotinylated primers (36, 36′ with 44′) immobilized within the depressions 12; first biotinylated transposome complexes (10 with biotin 5′ end functional groups 22) immobilized within the depressions 12, the first biotinylated transposome complexes including a first amplification domain 26; and second biotinylated transposome complexes (10′ with biotin 5′ end functional groups 22′) immobilized within the depressions 12, the second biotinylated transposome complexes including a second amplification domain 26′.

In another example, the streptavidin 46 may be introduced into the flow cell before the biotinylated transposome complexes 10, 10′ are introduced into the flow cell. The streptavidin 46 may be introduced into the flow cell at room temperature and binding of the streptavidin 46 to the biotin 44 of the polymeric hydrogel 24′ may take place in less than 30 minutes. The biotinylated transposome complexes 10, 10′ may then be introduced into the flow cell and allowed to incubate to attach to the pre-assembled streptavidin 46.

In still another example, the streptavidin 46 may be mixed with the biotinylated transposome complexes 10, 10′ before they are introduced into the flow cell. The streptavidin 46 and biotinylated transposome complexes 10, 10′ may be allowed to incubate at room temperature for about 30 minutes or less. This will pre-assemble the streptavidin onto the biotinylated transposome complexes 10, 10′. The streptavidin-biotinylated transposome complexes 10, 10′ may then be introduced into the flow cell and allowed to incubate to attach to the biotin of the polymeric hydrogel 24′.

In still another example, streptavidin 46 and biotin 44 are pre-attached to one another to form a streptavidin-biotin bound pair, and then the biotin 44 of the streptavidin-biotin bound pair is attached to the surface of the polymeric hydrogel 24 through some of the R^{A} groups (i.e., the azide, tetrazine, or other functional group that can attach to an alkyne). The biotin 44 of the streptavidin-biotin bound pair may be attached to a linker, such as bicyclo[6.1.0]nonyne (BCN), which can covalently attach to some of the R^{A} groups. In this example, biotinylated transposome complexes 10, 10′ may be introduced into the flow cell containing the streptavidin-biotin bound pair as part of the polymeric hydrogel 24′.

In still other examples, the transposome complexes 10, 10′ may be fully assembled with biotin-streptavidin-biotin, where biotin is the linker 22, 22′ attached to streptavidin 46 attached to another biotin 44. In this example, the polymeric hydrogel 24 is used, which includes surface groups or surface bound linkers, such as bicyclo[6.1.0]nonyne (BCN), that can attach to the biotin 44 of the fully assembled transposome complexes 10, 10′.

One specific example of the flow cell shown in FIG. 1A, FIG. 2A, and FIG. 2B comprises a substrate 40 having depressions 12 separated by interstitial regions 38; first and second primers 36, 36′ immobilized within the depressions 12; first transposome complexes 10 immobilized within the depressions 12, the first transposome complexes including a first amplification domain 26; and second transposome complexes 10′ immobilized within the depressions 12, the second transposome complexes 10′ including a second amplification domain 26′; wherein i) some of the first transposome complexes 10, or ii) some of the second transposome complexes 10′, or iii) some of both of the first and second transposome complexes 10, 10′ include a modification to reduce tagmentation efficiency. Any of the transposome complex modifications described herein may be used, and any of the attachment mechanisms described in reference to FIG. 1A, FIG. 2A, and FIG. 2B may be used.

Other examples of the flow cell include the transposome complexes 10, 10′ attached to the substrate surface in spatially separated arrangements. Some examples are shown in FIG. 3A, FIG. 3B, FIG. 4, and FIG. 5. These examples of the flow cell include a substrate 40 having depressions 12 separated by interstitial regions 38; first and second primers 36, 36′ immobilized within the depressions 12; first transposome complexes 10 including a first amplification domain 26; second transposome complexes 10′ including a second amplification domain 26′; wherein one of: i) the first and second transposome complexes 10, 10′ are respectively immobilized at different regions A, B of the depressions 12 (FIG. 3A and FIG. 3B); or ii) the first and second transposome complexes 10, 10′ are respectively immobilized within the depressions 12 and on the interstitial regions 38 (FIG. 4); or iii) the first and second transposome complexes 10, 10′ are respectively immobilized on different areas C, D of the interstitial regions 38 (FIG. 5).

In the example shown in FIG. 3A, the polymeric hydrogel 24 includes two regions A, B. One region A has the first primers 36 and the first transposome complexes 10 attached thereto, and the other region B has the second primers 36′ and the second transposome complexes 10′ attached thereto.

In one example, the polymeric hydrogel 24 is chemically the same throughout the regions A, B, and suitable techniques may be used to immobilize the primers 36, 36′ and transposome complexes 10, 10′ to the respective regions A, B. For example, the polymeric hydrogel 24 across both regions A, B may include azides and/or tetrazines, which can attach to alkynes (e.g., BCN). One example of a suitable technique may include the use of a photoresist. In this example, the photoresist is developed to mask one region A while the surface chemistry (e.g., primers 36, 36′ and transposome complexes 10, 10′) is added to the other region B. The photoresist is then removed, and the surface chemistry to the region A under conditions that will not deleteriously affect the region B or its surface chemistry. Another example of a suitable technique may include the use of a sacrificial layer, such as aluminum. In this example, the sacrificial layer is applied to the substrate 40 so that it masks a portion (e.g., one half) of the depression 12. The other portion (e.g., other half) of the depression 12 remains exposed. The exposed portion of the depression may be activated, e.g., by depositing a suitable silane (e.g., depositing norbornene silane using CVD). In this example, the polymeric hydrogel 24, A is applied over the sacrificial layer and over the exposed and activated portion of the depression 12. The sacrificial layer is then lifted off, which removes the overlying polymeric hydrogel 24 and exposes the previously covered portion of the depression 12. The polymeric hydrogel 24, A on the other portion of the depression 12 remains intact. The primers 36 and transposome complexes 10 may then be grafted to the polymeric hydrogel 24, A. In one example, primer 36 grafting involves azide reduction of the polymeric hydrogel 24, A, followed by application of an NHS-tetrazine agent, followed by grafting of BCN-terminated transposome complexes 10 and BCN-terminated primers 36. The polymeric hydrogel 24, A may be exposed to a capping reagent to deactivate any unreacted tetrazine groups. Because the substrate 40 has no affinity for the primers 36 and complexes 10, the exposed portion of the depression 12 is not affected. The substrate 40 may then be activated, e.g., by exposure to a solution of norbornene silane. The polymeric hydrogel 24, B may then be applied under conditions (e.g., under high ionic strength (e.g., in the presence of 10x PBS, NaCl, KCI, etc.)) that will not deleteriously affect the region A or its surface chemistry. The primers 36′ and transposome complexes 10′ may then be grafted to the polymeric hydrogel 24, B. Still another example of a suitable technique may include pre-grafting the primers 36, 36′ and transposome complexes 10, 10′ and sequentially and selectively applying the pre-grafted polymeric materials to the regions A, B. It is to be understood that while several example methods have been provided, any other methods may be used to immobilize the primers 36, 36′ and transposome complexes 10, 10′ to the respective regions A, B.

In still another example, the regions A, B of the polymeric hydrogel 24 may be chemically different (i.e., orthogonal so that one region A can graft primers 36 and the other region B can graft primers 36′). In one example, the biotinylated polymeric hydrogel 24 may be applied as region A and the azide or tetrazine terminated polymeric hydrogel 24 may be applied as region B for respective primer 36, 36′ and transposome complex 10, 10′ attachment. In another example, the polymeric hydrogel 24 in region A has tetrazine terminal groups and the polymeric hydrogel 24 in region B has alkyne (non-BCN) terminal groups. In this example, primers 36 and complexes 10 terminated with trans-cyclooctene (TCO) and primers 36′ and complexes 10′ terminated with picolyl azide can simultaneously and respectively be grafted to the regions A and B of the polymeric hydrogel 24. In still another example, the polymeric hydrogel 24 in region A has tetrazine terminal groups and the polymeric hydrogel 24 in region B has dibenzocyclooctyne terminal groups. In this example, primers 36 and complexes 10 terminated with trans-cyclooctene (TCO) and primers 36′ and complexes 10′ terminated with picolyl azide can simultaneously and respectively be grafted to the regions A and B of the polymeric hydrogel 24. While several examples have been provided, it is to be understood that any combination of orthogonal chemistries may be used at the regions A and B in order to respectively graft the primers 36 and 36′.

In the example shown in FIG. 4, the polymeric hydrogel 24 includes two regions A, B. One region A has the first primers 36 and the first transposome complexes 10 attached thereto, and the other region B has the second primers 36′ attached thereto. In this example, the second transposome complexes 10′ are attached to the interstitial regions 38. The methods described in reference to FIG. 3A may be used to attach the primers 36 and the first transposome complexes 10 to the region A and to attach the primers 36′ to the region B. In this example, the interstitial regions 38 may have material 48 selectively added thereto that is capable of attaching the second transposome complexes 10′ thereto. Selective deposition techniques or salt exclusion methods may be used to selectively apply the material 48 on the interstitial regions 38.

In the example shown in FIG. 5, the polymeric hydrogel 24 includes two regions A, B. One region A has the first primers 36 attached thereto, and the other region B has the second primers 36′ attached thereto. In this example, the first and second transposome complexes 10, 10′ are attached to areas of the interstitial regions 38. The methods described in reference to FIG. 3A may be used to attach the primers 36 to the region A and to attach the primers 36′ to the region B. The linkers 22, 22′ of the transposome complexes 10, 10′ may be selected so that they attach to the material 48 applied to the interstitial regions 38 or to the substrate 40 (if the material 48 is not used).

Any examples of the primers 36, 36′ and transposome complexes 10, 10′ disclosed herein may be used in the flow cells described in reference to FIG. 3A, FIG. 4, and FIG. 5.

In still another example, the spatially separated arrangement of the transposome complexes 10, 10′ is achieved using a multi-depth depression 12′. This depression 12′ is shown in FIG. 3B. In addition to enabling spatial separation, the use of this particular depression 12′ may also lead to increased insert sizes.

The multi-depth depression 12′ includes a shallow portion 84 and a deep portion 86. The depths of the shallow portion 84 and the deep portion 86 are within the ranges set forth herein for the depression 12, with the caveat that the deep portion 86 has a greater depth than the shallow portion 84.

As shown in FIG. 3B, the polymeric hydrogel 24 is applied on the surfaces within the multi-depth depression 12′. While not shown in FIG. 3B, it is to be understood that the polymeric hydrogel 24 may be conformally coated along all of the surfaces (including the bottom surfaces and the sidewalls) within the multi-depth depression 12′, as long as the interstitial regions 38 remain free of the polymeric hydrogel 24. Any example of the polymeric hydrogel 24 described herein may be used in this example.

As shown in FIG. 3B, a first primer set (including a cleavable first primer 36C₁ and an uncleavable second primer 36D₁) is immobilized on a surface in the shallow portion 84; first transposome complexes 10 are immobilized on the surface in the shallow portion 84, the first transposome complexes 10 including the first amplification domain 26 as described herein; a second primer set (an uncleavable first primer 36C₂ and a cleavable second primer 36D₂) immobilized on a surface in the deep portion 86; and second transposome complexes 10′ are immobilized on the surface in the deep portion 86, the second transposome complexes 10′ including a second amplification domain 26′ as described herein. The cleavable first primer 36C₁, uncleavable second primer 36D₁, uncleavable first primer 36C₂ and cleavable second primer 36D₂ are described in more detail in reference to FIG. 17. In these examples, the first amplification domain 26 has the same sequence, e.g., as the uncleavable first primer 36C₂ and the cleavable first primer 36C₁ and the second amplification domain 26 has the same sequence, e.g., as the cleavable second primer 36D₂ and the uncleavable second primer 36D₁.

The primers 36C₁, 36D₁, 36C₂, 36D₂ and the transposome complexes 10, 10′ include 5′ functional groups that enable them to be attached to the polymeric hydrogel 24. Any of the examples set forth herein may be used, such as a terminal alkyne (e.g., hexynyl) or an internal alkyne, where the alkyne is part of a cyclic compound (e.g., bicyclo[6.1.0]nonyne (BCN)).

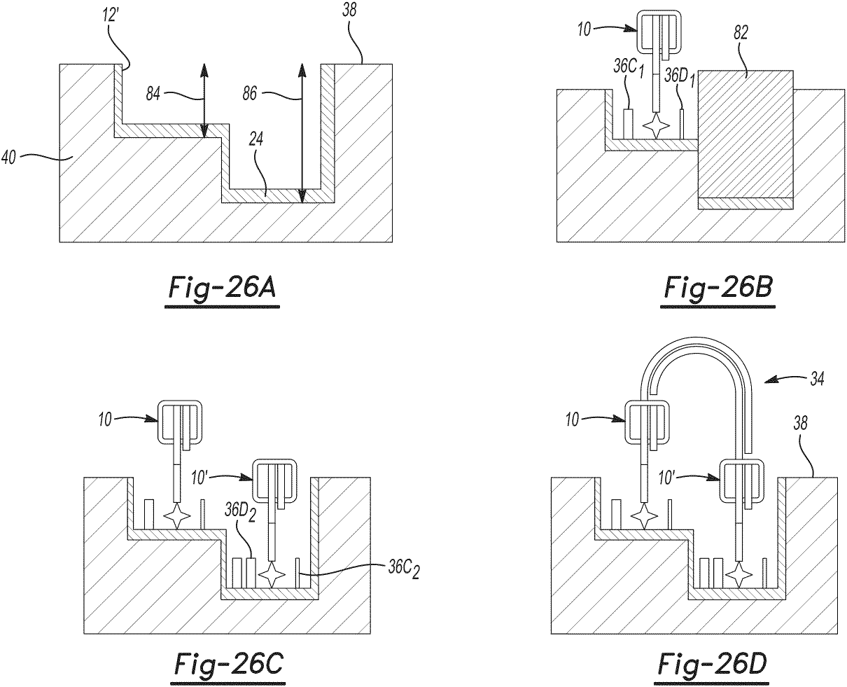

An example of method for making the configuration shown in FIG. 3B is schematically depicted in FIG. 26A through FIG. 26D. While a single multi-depth depression 12′ is shown, it is to be understood that the method may generate an array of the multi-depth depressions 12′ in the substrate 40.

The method generally includes applying the polymeric hydrogel 24 over the substrate 40 having multi-depth depressions 12′ separated by interstitial regions 38, where each multi-depth depression 12′ includes the deep portion 86 and the shallow portion 84 (FIG. 26A); removing the polymeric hydrogel 24 from the interstitial regions 38 (FIG. 26B); developing a photoresist to generate an insoluble photoresist 82 in the deep portion 86, whereby the shallow portion 84 remains exposed (FIG. 26B); while the insoluble photoresist 82 is in the deep portion 86, immobilizing a first primer set and first transposome complexes 10 on a surface in the shallow portion 84 (FIG. 26B); removing the insoluble photoresist (FIG. 26C); and immobilizing a second primer set and second transposome complexes 10′ on a surface in the shallow portion 84 (FIG. 26C).

At the outset of the method, the multi-depth depression 12′ is formed in the substrate 40. The multi-depth depression 12′ may be formed using nanoimprint lithography. As an example, a working stamp is pressed into the substrate 40 (e.g., the single layer base support or the layer positioned on a base support) while the substrate material is soft, which creates an imprint (negative replica) of the working stamp features in the substrate 40. The substrate 40 may then be cured with the working stamp in place.

Curing may be accomplished by exposure to actinic radiation, such as visible light radiation or ultraviolet (UV) radiation, when a radiation-curable resin material is used; or by exposure to heat when a thermal-curable resin material is used. Curing may promote polymerization and/or cross-linking. As an example, curing may include multiple stages, including a softbake (e.g., to drive off any liquid carrier that may be used to deposit the resin) and a hardbake. The softbake may take place at a lower temperature, ranging from about 50° C. to about 150° C., for greater than 0 seconds to about 3 minutes. The duration of the hardbake may last from about 5 seconds to about 10 minutes at a temperature ranging from about 100° C. to about 300° C. Examples of devices that can be used for softbaking and/or hardbaking include a hot plate, oven, etc.

After curing, the working stamp is released. This creates topographic features in the substrate 40.

The polymeric hydrogel 24 is then applied over the substrate 40. The polymeric hydrogel 24 may be applied using any suitable deposition technique. When first applied, the polymeric hydrogel 24 conformally coats the entire substrate 40 including the surfaces of the multi-depth depression 12′ as well as the interstitial regions 38.

The polymeric hydrogel 24 that is positioned over the interstitial regions 38 is removed, e.g., using a polishing process. The polishing process may be performed with a chemical slurry (including, e.g., an abrasive, a buffer, a chelating agent, a surfactant, and/or a dispersant) which can remove the polymeric hydrogel 24 from the interstitial regions 38 without deleteriously affecting the underlying substrate 40 at those regions 38. Alternatively, polishing may be performed with a solution that does not include the abrasive particles. The chemical slurry may be used in a chemical mechanical polishing system to polish the surface of the interstitial regions 38. The polishing head(s)/pad(s) or other polishing tool(s) is/are capable of polishing the polymeric hydrogel 24 that may be present over the interstitial regions 38 while leaving the polymeric hydrogel 24 in the multi-depth depression(s) 12′ at least substantially intact. As an example, the polishing head may be a Strasbaugh ViPRR II polishing head. Cleaning and drying processes may be performed after polishing. The cleaning process may utilize a water bath and sonication. The water bath may be maintained at a relatively low temperature ranging from about 22° C. to about 30° C. The drying process may involve spin drying, or drying via another suitable technique.

The polymeric hydrogel 24 in the multi-depth depression 12′ is shown in FIG. 26A.

A photoresist is then applied over the polymeric hydrogel 24. The photoresist may be a positive photoresist or a negative photoresist. Examples of suitable positive photoresists include the MICROPOSIT® S1800 series or the AZ® 1500 series, both of which are available from Kayaku Advanced Materials, Inc. Another example of a suitable positive photoresist is SPR™-220 (from DuPont). Examples of suitable negative photoresists include the SU-8 Series and the KMPR® Series (both of which are available from Kayaku Advanced Materials, Inc.), the UVN™ Series (available from DuPont), or the NR® series photoresist (available from Futurrex). The positive or negative photoresist may be applied using any suitable deposition technique disclosed herein.

When the positive photoresist is used, the portion of the positive photoresist in the deep portion 86 is not exposed to certain wavelengths of light to form the insoluble photoresist 82. Those portions that are exposed to the light remain soluble and can be removed with a suitable developer. Examples of suitable developers for the positive photoresist 38 include aqueous-alkaline solutions, such as diluted sodium hydroxide, diluted potassium hydroxide, or an aqueous solution of the metal ion free organic TMAH (tetramethylammoniumhydroxide).

When the negative photoresist is used, the portion of the negative photoresist in the deep portion 86 is exposed to certain wavelengths of light to form the insoluble photoresist 82. Those portions that are not exposed to the light remain soluble and can be removed with a suitable developer. Examples of suitable developers for the negative photoresist include aqueous-alkaline solutions, such as diluted sodium hydroxide, diluted potassium hydroxide, or an aqueous solution of the metal ion free organic TMAH (tetramethylammoniumhydroxide).

In either example, a mask may be used to selectively expose the photoresist to the light in the desired manner. When the negative photoresist is used, backside light exposure (e.g., through the substrate 40) may be used as long as light is transmitted through the substrate 40 to reach the deep portion 86 and is blocked from transmitting through the substrate 40 to reach the shallow portion 84. In this particular example, the thicknesses of the substrate 40 at the deep portion 86 and at the shallow portion 84 may be selected to be light transmissive and light blocking, respectively.

Upon removal of the soluble photoresist, the interstitial regions 38 and the polymeric hydrogel 24 in the shallow portion 84 are exposed and the insoluble photoresist 82 remains in the deep portion, as shown in FIG. 26B.

While the insoluble photoresist 82 is in the deep portion 86, the first primer set and the first transposome complexes 10 are immobilized on the surface of the polymeric hydrogel 24 in the shallow portion 84 (FIG. 26B). For immobilization, the transposome complexes 10 and the primers 36C₁, 36D₁ may be included in a carrier liquid in a concentration ranging from about 0.1 µM to about 1 µM. The carrier liquid may be water. A buffer and/or salt may also be added to the carrier liquid for grafting the transposome complexes 10 and the primers 36C₁, 36D₁ to suitable functional groups of the polymeric hydrogel 24. The buffer has a pH ranging from 5 to 12. Any of the neutral buffers and/or salts set forth herein may be used.

The fluid containing the transposome complexes 10 and the primers 36C₁, 36D₁ may be introduced using flow through deposition. Grafting may be performed at a temperature ranging from about 35° C. to about 45° C. for a time ranging from about 30 minutes to about 120 minutes. During grafting, the transposome complexes 10 and primers 36C₁, 36D₁ attach to at least some of the azide or tetrazine groups of the polymeric hydrogel 24 and have no affinity for the interstitial regions 38.

The washing solution may be flowed through the flow cell to remove any ungrafted transposome complexes 10 and/or primers 36C₁, 36D₁.

The insoluble photoresist 82 is then removed. The insoluble positive photoresist may be lifted off with removers, such as dimethylsulfoxide (DMSO) with sonication, an acetone wash, a propylene glycol monomethyl ether acetate wash, or an NMP (N-methyl-2-pyrrolidone) based stripper wash. The insoluble negative photoresist may be lifted off with removers, such as dimethylsulfoxide (DMSO) with sonication, or acetone, or an NMP (N-methyl-2-pyrrolidone) based stripper.

The second primer set and second transposome complexes 10′ are then immobilized on the surface in the deep portion 86, as shown in FIG. 26C. For immobilization, the transposome complexes 10′ and the primers 36C₂, 36D₂ may be included in a carrier liquid in a concentration ranging from about 0.1 µM to about 1 µM. The carrier liquid containing the transposome complexes 10′ and the primers 36C₂, 36D₂ may be water, and may include a buffer and/or salt. This carrier fluid may be introduced to the flow cell using flow through deposition, and grafting may be performed as described herein.

The second primer set and second transposome complexes 10′ will be substantially blocked from grafting to the polymeric hydrogel 24 in the shallow portion 84 due to the presence of the transposome complexes 10 and the primers 36C₁, 36D₁.

The washing solution may be flowed through the flow cell to remove any ungrafted transposome complexes 10′ and primers 36C₂, 36D₂.

A DNA sample 34 may then be introduced into the flow cell and exposed to tagmentation and extension to generate fully adapted DNA fragments. The fragments can be cleaved from the transposome complexes 10, 10′, seeded by one of the primers 36C₁, 36D₁, 36C₂, 36D₂ and exposed to amplification across both of the primer sets. Amplification generates forward and reverse strands in both the deep portion 86 and the shallow portion 84. However, the cleavage site in each of the cleavable primers 36C₁, 36D₂ (described in reference to FIG. 17) enables the forward strands to cleaved from either the deep or shallow portion 86 or 84, and reverse strands to be cleaved from the other of the shallow or deep portion 84 or 86. Thus, reverse strands remain in the portion 86 or 84 and the forward strands remain in the portion 84 or 86. This enables simultaneous paired end sequencing.

In the example shown in FIG. 3A, cluster generation may form three different types of bridged and fully extended (fully adapted) fragment amplicons. The bridged and fully adapted fragment amplicons formed on the polymeric hydrogel 24, A have the same amplification domain 26 at both ends (e.g., P5-P5) due to the same transposomes 10 and the same primers 36 being present on the polymeric hydrogel 24, A. Similarly, the fully adapted fragment amplicons formed on the polymeric hydrogel 24, B have the same amplification domain 26′ at both ends (e.g., P7-P7) due to the same transposomes 10′ and the same primers 36′ being present on the polymeric hydrogel 24, B. In contrast, the fully adapted fragment amplicons formed on both the polymeric hydrogels 24, A and 24, B (i.e., those that bridge the hydrogels 24, A and 24, B) have the different amplification domains 26, 26′ at the opposed ends (e.g., P5-P7). This is because tagmentation and amplification taking place across both polymeric hydrogels 24, A and 24, B involve both transposomes 10, 10′ and both primers 36, 36′.

Due to the orthogonal cleavage groups in the amplification domains 26, 26′ of the fully adapted fragment amplicons, when specific base cleavage is performed, one type of bridged and fully adapted fragment amplicons (e.g., those formed on the polymeric hydrogel 24, A) are removed while the other type of bridged and fully adapted fragment amplicons (e.g., those formed on the polymeric hydrogel 24, B) remain bridged. As an example, specific base cleavage targets the cleavage group in the amplification domain 26. In this example, specific base cleavage also linearizes the fully adapted fragment amplicons having different amplification domains 26, 26′ at the opposed ends, which in this example, leaves them attached to the polymeric hydrogel 24, B. These linearized fully adapted fragment amplicons are sequenced and then the sequenced products are removed.

Clustering is then performed again with the previously linearized fully adapted fragment amplicons (e.g., those formed on the polymeric hydrogel 24, B) and the primers 36 on the other polymeric hydrogel 24, A. The fully adapted fragment amplicons that remain bridged after the first round of sequencing (e.g., those formed on the polymeric hydrogel 24, B) do not participate in clustering. In this example, specific base cleavage is performed so that the remaining bridged and fully adapted fragment amplicons (e.g., those formed on the polymeric hydrogel 24, B) are removed. In this specific example, specific base cleavage targets the cleavage group in the amplification domain 26′. This specific base cleavage linearizes the newly generated and fully adapted fragment amplicons having different amplification domains 26, 26′ at the opposed ends, which in this example, leaves them attached to the polymeric hydrogel 24, A. These linearized fully adapted fragment amplicons are sequenced and then the sequenced products are removed.

In the example of FIG. 3A, the intensity during sequencing may be affected because the fully adapted fragments having different amplification domains 26, 26′ are limited to the area that spans the two polymeric hydrogels 24, A and 24, B. Another example of the flow cell and method may be used to increase the number of fully adapted fragments having different amplification domains 26, 26′ at opposed ends that are formed. This example flow cell is shown in FIG. 15A and the example method is schematically depicted from FIG. 15A to FIG. 15J.

The flow cell shown in FIG. 15A includes the substrate 40 having depressions 12 separated by interstitial regions 38; a first polymeric hydrogel 24, A positioned in a first portion of each depression 12; a second polymeric hydrogel 24, B positioned in a second portion of each depression 12; a transposome precursor 52 immobilized on the first polymeric hydrogel 24, A via an orthogonally cleavable linkage 54; a capture primer 56 immobilized on the second polymeric hydrogel 24, B; and first and second primers 36C, 36D of an amplification primer set immobilized on each of the first and second polymeric hydrogels 24A, 24B.

The substrate 40 may be any of the examples set forth herein.

The primers 36C, 36D of the amplification primer set may be any of the examples set forth herein for the primers 36, 36′, as long as they have orthogonal cleavage sites. In other examples, described further in reference to FIG. 17, each of the first and second primers of the amplification primer set includes both a cleavable primer and an uncleavable primer.

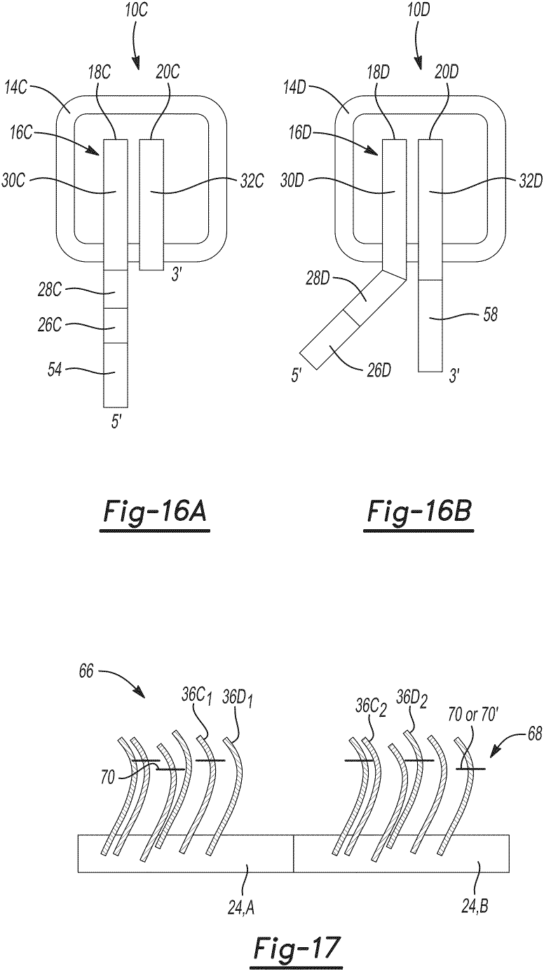

The capture primers 56 are capable of hybridizing to a pre-assembled transposome complex 10D, but that do not otherwise participate in amplification because they are orthogonal to the primers 36C, 36D in the amplification primer set. As will be discussed in further detail herein, the capture primer includes a sequence that is complementary to a non-transferred strand 20D (FIG. 15B and FIG. 16B) of a pre-assembled transposome complex 10D. As one example, the sequence of the capture primers 56 (PX) may be:

PX 5′ → 3′

AGGAGGAGGAGGAGGAGGAGGAGG (SEQ. ID. NO. 10)

The capture primers 56 may also include the polyT sequence at the 5′ end of the primer sequence. The capture primers 56 do not include a cleavage site.

The transposome precursor 52 includes, from its 5′ end to its 3′ end: an orthogonally cleavable linkage 54, an amplification domain 26C, a sequencing primer sequence 28C, and a strand 30C that is capable of forming a transposon end 16C (see FIG. 16A). As shown in FIG. 16A, the transposome precursor 52 becomes the transferred strand 18C of a transposome complex 10C that is assembled on the flow cell surface.

The orthogonally cleavable linkage 54 has orthogonal cleavage chemistry to the primers 36C, 36D of the amplification primer set. As such, the orthogonally cleavable linkage 54 is not susceptible to the cleaving agents used for either of the cleavage sites of the primers 36C, 36D, and the cleavage sites of the primers 36C, 36D are not susceptible to the cleaving agent used for orthogonally cleavable linkage 54. In addition to containing a cleavage site, the orthogonally cleavable linkage 54 is bi-functional, in that it is capable of covalently attaching, at its 3′ end, to the amplification domain 26C and is capable of covalently or non-covalently attaching, at its 5′ end, to surface functional groups of the polymeric hydrogel 24, A. The cleavage site of the orthogonally cleavable linkage 54 may be vicinal diol linkages (that can be cleaved by oxidation, such as treatment with a periodate reagent), disulfide linkages (cleavable, for example, under reducing conditions (e.g., in the presence of dithiothreitol (DTT)), or in the presence of a phosphine), ortho-nitrobenzyl groups (cleavable, for example, by photolysis), azobenzene linkages (cleavable, for example, in the presence of Na₂S₂O₄), alkyl-selenium linkages (cleavable, for example, by oxidation, such as treatment with hydrogen peroxide), silyl ether linkages (cleavable, for example, by fluoride ion, acid, or base), or allyl carbamate linkages (cleavable, for example, in the presence of a palladium complex). Some examples of the orthogonally cleavable linkage 54 include an aliphatic linker with a vicinal diol, a poly(ethylene glycol) linker with a disulfide, or the like.

The transposome precursor 52 also includes the amplification domain 26C. The amplification domain 26C of the transposome precursor 52 has the same sequence as the first primer 36C, except that the amplification domain 26C does not include the cleavage site of the first primer 36C.

The transposome precursor 52 also includes the sequencing primer sequence 28C. The sequencing primer sequence 28C is capable of binding to a sequencing primer that introduced into the flow cell after tagmentation and amplification.

The transposome precursor 52 also includes the strand 30C. The strand 30 is capable of forming a transposon end 16C with a strand 32C that is subsequently introduced into the flow cell with a transposase 14C (see FIG. 15B). As such, the strand 30C is complementary with the strand 32C. Any examples of the strand 30 may be used for the strand 30C.

Similar to the example shown in FIG. 3A, the polymeric hydrogel 24 includes two regions A, B. One region A has the transposome precursors 52 and the primers 36C, 36D attached thereto, and the other region B has the capture primers 56 and the primers 36C, 36D attached thereto.

In one example, the polymeric hydrogel 24 is chemically the same throughout the regions A, B. In this example, the polymeric hydrogel 24 may be applied as described herein in reference to FIG. 1A through FIG. 1D, e.g., using blanket deposition, and then polishing to remove the polymeric hydrogel 24 from the interstitial regions 38. In this example, blanket grafting techniques may be used to immobilize the primers 36C, 36D to both regions A, B or the primers 36C, 36D may be pre-grafted to the polymeric hydrogel 24 before it is applied to the depression 12. Techniques may then be used to selectively immobilize the transposome precursor 52 to the region A and the capture primers 56 to the region B. Suitable selective immobilization techniques are described in reference to FIG. 3A. In the examples where the polymeric hydrogel 24 is chemically the same throughout the regions A, B, the 5′ end functional groups of the primers 36C, 36D and the 5′ end functional group of the orthogonally cleavable linkage 54 may be the same, as long as the cleavage sites of the entities 36C, 36D, 54 are orthogonal.

In another example, the polymeric hydrogel 24 is chemically different at the regions A, B. In this example, one region A is configured to graft some of the primers 36C, 36D and the transposome precursor 52, and the other region B is configured to graft some other of the primers 36C, 36D and the capture primers 56. Thus, some of the primers 36C, 36D may have different 5′ end functional groups than some other of the primers 36C, 36D in order to achieve the desired attachment to the chemically different regions A, B. Any of the covalent or non-covalent attachment mechanisms set forth herein may be used, as long as some of the primers 36C, 36D and the transposome precursor 52 are attached to the polymeric hydrogel 24, A, and some other of the primers 36C, 36D and the capture primers 56 are attached to the polymeric hydrogel 24, B. In this example, some of the primers 36C, 36D and the transposome precursor 52 may be pre-grafted to the polymeric hydrogel 24, A, and the polymeric hydrogel 24, A may be selectively applied to a first portion of the depression 12; and the other of the primers 36C, 36D and the capture primers 56 may be pre-grafted to the polymeric hydrogel 24, B, and the polymeric hydrogel 24, B may be selectively applied to a second portion of the depression 12. Alternatively, the polymeric hydrogels 24, A and 24, B may be selectively applied, and then the attachment of the primers 36C, 36D, the transposome precursor 52, and the capture primers 56 may take place simultaneously, as they are configured to respectively attach to the regions A and B of the polymeric hydrogel 24.

FIG. 15B depicts the on flow cell assembly of the transposome complex 10C and the on flow cell attachment of the pre-assembled transposome complex 10D.

The on flow cell assembly of the transposome complex 10C may be accomplished by introducing a first fluid, including a plurality of transposase enzymes 14C and a plurality of non-transferred strands 20C, to the flow cell shown in FIG. 15A.

In the first fluid, the transposase enzymes 14C may be any of the examples set forth herein for transposases 14, 14′. Also in the first fluid, the non-transferred strand 20C is the strand 32C, which is capable of forming the transposon end 16C with the strand 30C that is bound to the polymeric hydrogel 24, A. As such, the non-transferred strand 20C, 32C is complementary with the strand 30C. Any examples of the strand 32 may be used for the strand 32C, 20C. The concentration of each of the transposase enzymes 14C and the non-transferred strand 20C, 32C in the first fluid may depend upon the number of the transposome precursors 52 bound to the polymeric hydrogel 24, A. An excess of each of the transposase enzymes 14C and the non-transferred strand 20C, 32C with respect to the transposome precursors 52 may be used to ensure a desired number of transposome complexes 10C are formed. Any non-reacted transposase enzymes 14C and the non-transferred strand 20C, 32C can be removed in a washing solution.

The first fluid also includes a carrier liquid, which may be water.

Once introduced into the flow cell of FIG. 15A, the first fluid is allowed to incubate in the flow cell so that at least some of the plurality of transposome precursors 52, the plurality of non-transferred strands 20C, 32C, and the plurality of transposase enzymes 14C interact to form a plurality of transposome complexes 10C immobilized on the polymeric hydrogel 24A. The first fluid is allowed to incubate at a temperature suitable for hybridization of the non-transferred strands 20C, 32C in the first fluid with the strands 30C (of the transposome precursor 52). The hybridized strands 30C, 32C form a transposon end 16C, to which the transposase enzyme 14C can non-covalently bind. The resulting transposome complex 10C is shown in both FIG. 15B and FIG. 16A.

As shown in detail in FIG. 16A, the transposome complex 10C includes a transferred strand 18C hybridized to the non-transferred strand 20C, 32C at the transposon end 16C. The transferred strand 18C includes the amplification domain 26C and the orthogonally cleavable linkage 54 (that is attached to the polymeric hydrogel 24, A). As mentioned, the amplification domain 26C has the same sequence as the first primers 36C, except for the cleavage sites. This enables the transposome complex 10C to participate in amplification using the primers 36C, 36D, and to be cleaved separately from the primers 36C, 36D.

After the on flow cell assembly of the transposome complex 10C, the pre-assembled transposome complex 10D can be attached to the polymeric hydrogel 24, B within the flow cell.

The pre-assembled transposome complex 10D is shown in FIG. 15B (after attachment to the polymeric hydrogel 24, B) and in FIG. 16B (before attachment to the polymeric hydrogel 24, B). The pre-assembled transposome complex 10D includes a transposase enzyme 14D non-covalently bound to a transposon end 16D. The transposon end 16D is a double-stranded nucleic acid strand, one strand 30D of which is part of a transferred strand 18D and the other strand 32D of which is part of a non-transferred strand 32D. Any example of the transposase enzyme 14, the strand 30, and the strand 32 may be used, respectively, for the transposase enzyme 14D, the strand 30D, and the strand 32D.

The transferred strand 18D includes a forked adapter that is attached to the strand 30D of the transposon end 16D. The forked adapter includes an amplification domain 26D and a sequencing primer sequence 28D. The forked adapter does not attach to the polymeric hydrogel 24, B. The amplification domain 26D has the same sequence as the second primers 36D, and the sequencing primer sequence 28D is capable of binding a sequencing primer during downstream sequencing.

The non-transferred strand 20D includes two strand portions, one of which is the strand 32D of the transposon end 16D and the other of which is strand 58. As depicted in FIG. 16B, the strand 58 is attached to the 3′ end of the strand 32D. The strand 58 has a sequence that is complementary to the capture primers 56, and thus provides the pre-assembled transposome complex 10D with a hybridization mechanism for attaching to the polymeric hydrogel 24, B having the capture primers 56 attached thereto.

The pre-assembled transposome complex 10D may be prepared in solution outside of the flow cell. At the outset, the strands 18D and 20D may be synthesized, and then the strand portions 30D and 32D are hybridized together to form the forked adapter. The transposase enzyme 14D is added which complexes with the transposon end 16D.

The pre-assembled transposome complexes 10D may be added to a carrier fluid to form a second fluid. The concentration of the pre-assembled transposome complexes 10D in the second fluid may depend upon the number of capture primers 56 attached to the polymeric hydrogel 24, B. The carrier liquid of the second fluid may be water. An excess of pre-assembled transposome complexes 10D with respect to the capture primers 56 may be used to ensure a desired number of transposome complexes 10D are attached. Any non-reacted pre-assembled transposome complexes 10D can be removed in a washing solution.

The on flow cell attachment of the pre-assembled transposome complex 10D may be accomplished by introducing the second fluid, including a plurality of the pre-assembled transposome complex 10D, to the flow cell after the transposome complexes 10C have been assembled. The second fluid is allowed to incubate at a temperature suitable for hybridization of the portion strands 58 (see FIG. 16B) with the capture primers 56. This attaches the transposome complexes 10D to the polymeric hydrogel 24, B as shown in FIG. 15B.

In another example method, the pre-assembled transposome complexes 10D can be attached to the polymeric hydrogel 24, B before the on flow cell assembly of the complexes 10C.

A DNA sample is then introduced into the flow cell of FIG. 15B. The DNA sample may be introduced with an example of the tagmentation buffer disclosed herein.

FIG. 15C illustrates the depression 12 after the DNA sample has been introduced. The DNA sample is fragmented into DNA fragments 35, 37, whose 5′ ends are respectively ligated to the 3′ end of one of the transferred strands 18C or 18D. More specifically, in this example, the 5′ ends of the DNA fragments 35A, 37A are ligated to respective 3′ ends of transferred strands 18C of the transposome complexes 10C; the 5′ ends of the DNA fragments 35B, 37B are ligated to respective 3′ ends of transferred strands 18D of the pre-assembled transposome complexes 10D; and the 5′ ends of the DNA fragments 35C, 37C are ligated to respective 3′ ends of each of the transferred strands 18D, 18C of the transposome complexes 10D, 10C. Thus, the DNA fragments 35A, 37A are attached to the polymeric hydrogel 24, A; the DNA fragments 35B, 37B are attached to the polymeric hydrogel 24, B; and the DNA fragments 35C, 37C bridge the polymeric hydrogels 24, A and 24, B.

Fragmentation and ligation may take place at a temperature at or above 30° C. In one example, the temperature may range from 30° C. to about 55° C.

As depicted in FIG. 15C, the 3′ ends of the DNA fragments 35A, 37A, 35B, 37B, 35C, 37C are not ligated to the 5′ ends of the non-transferred strands 20C, 20D. As such, a gap 39 exists between the 3′ end of each of the DNA fragment strands 35A, 35B, 35C and the 5′ end of the non-transferred strand 20C or 20D, and a gap 39′ exists between the 3′ end of the DNA fragment strand 37A, 37B, 37C and the 5′ end of the non-transferred strand 20D or 20C. In one example, each gap 39, 39′ is nine (9) base pairs long.

The method then includes removing the plurality of transposase enzymes 14C and the transposase enzyme 14D of each of the plurality of pre-assembled transposome complexes 10D. Transposase enzyme 14C, 14D removal may be accomplished, for example, using sodium dodecyl sulfate (SDS) or proteinase or another denaturant. FIG. 15D illustrates the flow cell depression 12 after transposase enzyme removal.

This example of the method then includes initiating a strand displacement reaction to generate complementary copies 60, 62 of the transferred strands 18C, 18D of the plurality of transposome complexes 10C and of the plurality of pre-assembled transposome complexes 10D. The flow cell after the strand displacement reaction is depicted in FIG. 15E.

For the strand displacement extension, a nucleotide mixture containing nucleotides and a strand displacing enzyme is introduced into the flow cell. The nucleotide mixture may also include a liquid carrier, such as water and/or an ionic salt buffer fluid, e.g., saline citrate at milli-molar to molar concentrations, sodium chloride, potassium chloride, phosphate buffered saline, etc., and other buffers, such as tris(hydroxymethyl)aminomethane (Tris or TRIS) or (4-(2-hydroxyethyl)-1-piperazineethanesulfonic acid) (HEPES). The liquid carrier may also include catalytic metal(s) intended for the strand displacement extension reaction, such as Mg²⁺, Mn²⁺, etc. A single catalytic metal or a combination of catalytic metals may be used, and the total amount may range from about 0.01 mM to about 100 mM.

The enzyme used in the strand displacement reaction extends the 3′ ends of the DNA fragment strands 35A, 37A, 35B, 37B, 35C, 37C with the nucleotides in the mixture, using the transferred strands 18C, 18D as the respective template strands. An example of a suitable strand displacing enzyme includes Bst DNA polymerase or Bsu DNA polymerase.

During the strand displacement reaction, the enzyme displaces the non-transferred strands 20C, 20D. As a result, the DNA sample fragments 35B, 37B, 35C, 37C become detached from the second polymeric hydrogel 24, B, as shown in FIG. 15E. This is due to the fact that the strands 58 of the non-transferred strand 20D, which had been hybridized to respective surface bound capture primers 56, have been displaced. Without the attachment to the capture primers 56, the DNA sample fragments 35B, 37B, 35C, 37C are no longer attached to the polymeric hydrogel 24, B. The non-transferred strands 20C of the transposome complexes 10C are also displaced during the strand displacement reaction.

The detached and displaced strands may then be exposed to a denaturation and wash process. In one example, the denaturation and wash is performed with a formamide solution. This process removes any strands that are not covalently attached to the polymeric hydrogel 24 from the flow cell.

While not shown in FIG. 15E, it is to be understood that the denaturation and wash process may also denature the bridged fragments 35A, 37A, 35B, 37B, 35C, 37C. Whether bridged or single stranded, after the strand displacement reaction and washing, as shown in FIG. 15E, the DNA sample fragments 35A, 37A, 37C remain attached to the polymeric hydrogel 24, A. This is due to the fact that these DNA sample fragments 35A, 37A, 37C are attached to the polymer 24 through the orthogonally cleavable linkage 54 and not through the capture primers 56. While a single remaining DNA sample fragment 37C is shown in FIG. 15E, it is to be understood that any of the DNA sample fragments 37C that bridge the regions A, B of the polymeric hydrogel 24 will remain attached to the flow cell surface through the attached transferred strands 18C. As such, at least one of the complementary copies 62 of the transferred strands 18D of the plurality of pre-assembled transposome complexes 10D remains attached to the first polymeric hydrogel 24, A through at least one of the transferred strands 18C of the plurality of transposome complexes 10C.

The method then includes hybridizing the at least one of the complementary copies 62 of the transferred strands 18D of the plurality of pre-assembled transposome complexes 10D to at least one of the second primers 36D on the first polymeric hydrogel 24, A or the second polymeric hydrogel 24, B. In FIG. 15G, the complementary copy 62 is hybridized to one of the second primers 36D on the polymeric hydrogel 24, B. Hybridization may be initiated by increasing the temperature of the flow cell, and thus its contents, to a suitable DNA hybridization temperature (e.g., 65° C.).

As shown in FIG. 15H, polymerase extension is then initiated along the DNA sample fragment 37C that is attached to the hybridized at least one of the complementary copies 62 to generate at least one amplicon 64 that is attached to the at least one of the second primers 36D on the first polymeric hydrogel 24, A, or the second polymeric hydrogel 24, B.

For polymerase extension, a nucleotide mixture containing nucleotides and a polymerase is introduced into the flow cell. Any polymerase that can accept the nucleotide, and that can successfully incorporate the base of the nucleotide at the 3′ end of the primer 36D may be used. Example polymerases include those polymerases from family A, such as Bsu Polymerase, Bst Polymerase, Taq Polymerase, T7 Polymerase, and many others; polymerases from families B and B2, such as Phi29 polymerase and other highly processive polymerases (family B2), Pfu Polymerase (family B), KOD Polymerase (family B), 9oN (family B), and many others; polymerases from family C, such as Escherichia coli DNA Pol III, and many others, polymerases from family D, such as Pyrococcus furiosus DNA Pol II, and many others; polymerases from family X, such as DNA Pol µ, DNA Pol β, DNA Pol σ, and many others. The nucleotide mixture may also include a liquid carrier, such as water and/or an ionic salt buffer fluid, e.g., saline citrate at milli-molar to molar concentrations, sodium chloride, potassium chloride, phosphate buffered saline, etc., and other buffers, such as tris(hydroxymethyl)aminomethane (Tris or TRIS) or (4-(2-hydroxyethyl)-1-piperazineethanesulfonic acid) (HEPES). The liquid carrier may also include catalytic metal(s) intended for the extension reaction, such as Mg²⁺, Mn²⁺, etc. A single catalytic metal or a combination of catalytic metals may be used, and the total amount may range from about 0.01 mM to about 100 mM.

The temperature of the flow cell may be adjusted to initiate the template extension reaction. The polymerase enables the extension of the 3′ end of the primer 36D using the complementary copy 62, the DNA fragment 37C, and the transferred strand 18C as the template.

After the amplicon(s) 64 is/are generated, a cleaving agent of the orthogonally cleavable linkage 54 is introduced into the cell. The cleaving agent may be a chemical cleaving agent or an enzymatic cleaving agent depending on the cleavage site of the orthogonally cleavable linkage 54. Cleavage of the orthogonally cleavable linkage 54 severs the transferred strands 18C, and thus detaches the DNA fragments 35A, 37A, 37C from the polymeric hydrogel 24, A. In one example, a diol cleaving agent is introduced to detach the DNA sample fragments 35A, 37A, 37C bound to the first polymeric hydrogel 24, A via a diol cleavage site of the linkage 54.

The detached strands 35A, 37A may then be washed from the flow cell using an example of the washing solution set forth herein.

After the cleavage of the orthogonally cleavable linkage 54 and washing, as shown in FIG. 15I, the DNA sample fragment 37C and its amplicon 64 remain attached to the polymeric hydrogel 24, B. This is due to the fact that the DNA sample fragment 37C is attached to the polymer 24 (at region A or B) through hybridization of the complementary portion 62 to the primer 36D, and not through the transferred strand 18C. While a single remaining DNA sample fragment 37C and amplicon 64 are shown in FIG. 15I, it is to be understood that any of the DNA sample fragments 37C and amplicons 64 hybridized to the primers 36D will remain after cleavage of the orthogonally cleavable linkage 54.

The amplicon 64 is a fully adapted fragment (including amplification domains 26C, 26D or complements thereof at opposed ends) that can undergo amplification across the depression 12 using the primers 36C, 36D. The fully adapted DNA sample fragment 37C shown in FIG. 15I (which also includes amplification domains 26C, 26D or complements thereof at opposed ends) can be removed via denaturation. The amplicon 64 is then exposed to clustering. In particular, the single stranded amplicon 64 loops over to hybridize to an adjacent, complementary primer 36C, and a polymerase copies the amplicon 64 to form another double stranded bridge, which is denatured to form two single stranded strands that are attached to the polymeric hydrogel 24 (A or B). These two strands loop over and hybridize to adjacent, complementary primers 36C, 36D and are extended again to form two new double stranded loops. The process is repeated on each template copy by cycles of isothermal denaturation and amplification to create dense clonal clusters, as shown in FIG. 15J. Each cluster of double stranded bridges is denatured. In an example, the reverse strands are removed by a cleaving agent suitable for the cleavage site of the primers 36C or 36D to which the reverse strands are attached (e.g., specific base cleavage), leaving forward template strands/amplicons. In another example, the forward strands are removed by a cleaving agent suitable for the cleavage site of the primers 36C or 36D to which the forward strands are attached (e.g., specific base cleavage), leaving reverse template strands/amplicons. Clustering results in the formation of several template strands immobilized in the depressions 12.

In the example shown in FIG. 15A through FIG. 15J, a gap exists between the polymeric hydrogel regions 24, A, B. When the gap is included, it imposes a lower limit on the size of the fragment amplicons that can be generated because at least one of the DNA fragments 35C, 37C bridges the gap. In other examples, the regions A, B of the polymeric hydrogel 24 may abut one another in the depression 12, and thus do not impose a lower limit on the size of the fragment amplicons that can be generated.

The amplification primer set described in reference to FIG. 15A through FIG. 15J enables sequential paired end sequencing. With sequential paired end sequencing, forward strands/fragment amplicons that are generated across both regions A, B of the polymeric hydrogel 24 can be sequenced and removed, and then reverse strands strands/fragment amplicons can be generated across both regions A, B of the polymeric hydrogel 24, sequenced and removed. In alternative examples, the amplification primer set enables simultaneous paired end sequencing. Examples of this primer set are shown and described in reference to FIG. 17.

The primer set shown in FIG. 17 includes two primer sub-sets 66, 68. These primer sub-sets 66, 68 allow a single template strand (e.g., DNA fragment 35C) to be amplified and clustered across both primer sub-sets 66, 68 (as described in reference to FIG. 15A through FIG. 15J), and also enable a cluster of forward strands to be generated in one region A of the polymeric hydrogel 24 and a cluster of reverse strands to be generated in another region B of the polymeric hydrogel 24. To enable the latter, the primer sub-sets 66, 68 are controlled so that the cleaving (linearization) chemistry is orthogonal at the different polymeric hydrogel regions 24, A and 24, B.

More specifically, the primer sub-sets 66, 68 are related in that one set 66 includes a cleavable first primer 36C₁ and an uncleavable second primer 36D₁ and the other set 68 includes an uncleavable first primer 36C₂ and a cleavable second primer 36D₂. As such, the un-cleavable first and second primers 36C₂, 36D₁ of the primer sets 68, 66 are uncleavable during linearization of the clusters.

The primer sub-set 66 includes a cleavable first primer 36C₁ and an uncleavable second primer 36D₁; and the primer sub-set 68 includes an uncleavable first primer 36C₂ and a cleavable second primer 36D₂.

The cleavable first primer 36C₁ and the uncleavable second primer 36D₁ are oligonucleotide pairs, e.g., where the cleavable first primer 36C₁ is a forward amplification primer and the uncleavable second primer 36D₁ is a reverse amplification primer or where the uncleavable second primer 36D₁ is the forward amplification primer and the cleavable first primer 36C₁ is the reverse amplification primer. In the primer sub-set 66, the cleavable first primer 36C₁ includes a cleavage site 70, while the uncleavable second primer 36D₁ does not include a cleavage site.

The uncleavable first primer 36C₂ and the cleavable second primer 36D₂ are also oligonucleotide pairs, e.g., where the uncleavable first primer 36C₂ is a forward amplification primer and the cleavable second primer 36D₂ is a reverse amplification primer or where the cleavable second primer 36D₂ is the forward amplification primer and the uncleavable first primer 36C₂ is the reverse amplification primer. In the primer set 68, the cleavable second primer 36D₂ includes a cleavage site 70 or 70′, while the uncleavable first primer 36C₂ does not include a cleavage site.

It is to be understood that the uncleavable first primer 36C₂ and the cleavable first primer 36C₁ have the same nucleotide sequence (e.g., both are forward amplification primers), except that the cleavable first primer 36C₁ includes the cleavage site 70 integrated into the primer sequence or into a linking molecule attached to the primer sequence. Similarly, the cleavable second primer 36D₂ and the uncleavable second primer 36D₁ have the same nucleotide sequence (e.g., both are reverse amplification primers), except that the cleavable second primer 36D₂ includes the cleavage site 70 or 70′ integrated into the primer sequence or into the linking molecule attached to the primer sequence.

It is to be understood that when the first primers 36C₁ and 36C₂ are forward amplification primers, the second primers 36D₁ and 36D₂ are reverse primers, and vice versa.

The uncleavable primers 36D₁, 36C₂ may be any primer sequence with a universal sequence for amplification purposes, such as the P5 and P7 primers, P15 and P7 primers, or any combination of the PA, PD, PC, PD primers (e.g., PA and PB or PA and PD, etc.), without the cleavage site 70, 70′ (e.g., U or “n” in some of the sequences set forth herein).

In contrast, the cleavable primers 36C₁, 36D₂ may be any of the primer sequences set forth herein, e.g., the P5 and P7 primers, the P15 and P7 primers, or other universal sequence primers (e.g., the PA, PB, PC, PD primers) with the respective cleavage sites 70, 70′ (e.g., U or “n” in some of the sequences set forth herein) incorporated into the primer sequence or into the linking molecule attached to the primer sequence. The cleavage site 70 may be same in each of the cleavable primers 36C₁, 36D₂, or the cleavage sites 70, 70′ may be different in each of the cleavable primers 36C₁, 36D₂, as long as the cleavage sites 70 or 70 and 70′ are orthogonal to the cleavage site of the orthogonally cleavable linker 54.

Additionally, the flow cell shown in FIG. 15A through FIG. 15J may utilize different attachment mechanisms for the pre-assembled transposomes 10D. In this example, the flow cell would not include the capture primers 56, but rather, the polymeric hydrogel 24, B would include a transposome capture functional group that the first polymeric hydrogel 24, A is free of (i.e., that the first polymeric hydrogel 24, A does not include. In one example, the polymeric hydrogel 24, B includes functional groups that are capable of covalently attaching to the 5′ end of the transposome complex 10D, and the polymeric hydrogel 24, A does not include these particular functional groups. In another example, the polymeric hydrogel 24, B is functionalized with biotin surface groups (i.e., is the biotinylated polymeric hydrogel 24′), and the polymeric hydrogel 24, A does not include biotin surface groups. The 5′ end functional of the transposome complex 10D may be any of the examples set forth herein (e.g., streptavidin, streptavidin-biotin, etc.) that can attach to the biotin surface groups.

### Methods for Increasing Insert Size

As mentioned herein, examples of the modified transposome complexes 10, 10′ (e.g., with modifications at the 3′ or 5′ non-transferred strands 32, 32′ or at the 3′ end of the transferred strands 18, 18′) may be used to increase the insert size of the fragmented products. The following description provides several other methods that can be used to increase the insert size. These methods may be used in conjunction with any of the transposome complexes 10, 10′ disclosed herein, modified or non-modified.

In one example, a method for increasing an insert size of a deoxyribonucleic acid (DNA) sample, includes introducing the DNA sample to any example of the flow cell disclosed herein; introducing a condensation agent or a tagmentation inhibitor with the DNA sample, the condensation agent being selected from the group consisting of a polycationic amine (e.g. spermidine, chitosan, protamine, etc.), polyethylene glycol, and a histone or the tagmentation inhibitor being selected from the group consisting a cation (Zn²+), a pH adjustor (e.g., glutamic acid), and a chromatin assembly kit; and incubating the DNA sample in the flow cell in the presence of the condensation agent or the tagmentation inhibitor, whereby tagmentation of the DNA sample takes place at some of the first transposome complexes 10 and the second transposome complexes 10′.

In these examples, the percentage of transposome complexes 10, 10′ that are involved with tagmentation is less than the percentage of transposome complexes 10, 10′ that are involved with tagmentation with the condensation agent or the tagmentation inhibitor is not present.

Spermidine, chitosan, and protamine are examples of the DNA condensation agents. In an example, from about 2 mM to about 10 mM of spermidine or chitosan or protamine may be added with the DNA sample. Spermidine, chitosan, and protamine are cationic polymers that create a compact, round structure when incorporated in with the DNA sample. The compact, round structure is effective in blocking some of the transposases 14, 14′ from the DNA sample. Thus, fewer transposases 14, 14′ can access the DNA sample for tagmentation. This increases the size of the DNA fragments resulting from tagmentation.

Polyethylene glycol, in the presence of sodium chloride, is another example of a DNA condensation agent. In an example, polyethylene glycol may be added to the DNA sample in an amount greater than 2% (by volume), with greater than 25 mM NaCl. In some examples, the polyethylene glycol may be added to the DNA sample in an amount greater than 5% (by volume), with greater than 25 mM NaCl. Polyethylene glycol acts as a crowding agent when incorporated in with the DNA sample, and thus is effective in blocking some of the transposases 14, 14′ from the DNA sample. Thus, fewer transposases 14, 14′ can access the DNA sample for tagmentation. This increases the size of the DNA fragments resulting from tagmentation.

Cations, such as Zn²⁺, or a pH adjuster, such as glutamic acid, may be added as a tagmentation inhibitor. These agents may be added to reduce the efficiency of tagmentation. In an example, the Zn²⁺ cations may be present in an aqueous solution at a concentration ranging from about 0.02 mM to about 5 mM. In another example, glutamic acid may be present in an aqueous solution at a concentration ranging from about 0.25 mM to about 100 mM.

A chromatin assembly kit includes reagents that can generate assembled chromatin in the presence of the DNA sample. The chromatin packs some of the DNA, thus preventing the transposases 14, 14′ from accessing the DNA sample for tagmentation. This increases the size of the DNA fragments resulting from tagmentation.

### Sample Preparation

As described herein, a DNA sample is introduced into the flow cell where tagmentation takes place. Several example methods disclosed herein involve the preparation of the DNA sample. One example method includes: at an active temperature of a protease in an inorganic salt free lysis buffer including a serine protease, less than 2 mM ethylenediaminetetraacetic acid, a chaotropic detergent, and water, exposing a sample selected from the group consisting of whole blood, a tissue sample, blood spots, and saliva to the inorganic salt free lysis buffer, thereby extracting deoxyribonucleic acids from the whole blood sample and generating a crude lysate; inactivating the protease in the crude lysate using heat or an inhibitor; and adding a chelator of the chaotropic detergent to the crude lysate to generate a complexed crude lysate.

In this example method, the sample is first exposed to the inorganic salt free lysis buffer at the temperature at which the protease in the inorganic salt free lysis buffer is active. During exposure to the lysis buffer gDNA is extracted from the white blood cells in the sample. In one example of the method, the volume of the sample ranges from about 20 µL to about 50 µL.

The inorganic salt free lysis buffer includes, and in some examples consists of, a protease (e.g., a serine protease, such as thermolabile proteinase K, trypsin, etc.), less than 2 mM ethylenediaminetetraacetic acid (EDTA), a chaotropic detergent, and water.

The protease (e.g., serine protease, such as proteinase K, thermolabile proteinase K or trypsin) is included in the inorganic salt free lysis buffer to digest proteins that may deleteriously affect the gDNA during extraction and to protect the extracted gDNA from nucleases. The temperature at which the protease is active will depend upon the protease that is used. Proteinase K is active at temperatures ranging from about 25° C. to about 65° C., and may be fully inactivated at about 80° C. Proteinase K can alternatively be inactivated when exposed to a protease inhibitor, such as tetrapeptidyl chloromethyl ketone (TCK). Thermolabile proteinase K is active at low temperatures ranging from about 20° C. to about 40° C., and may be fully inactivated at about 55° C. Trypsin is active at temperatures ranging from about 4° C. to about 65° C., and can be inactivated when exposed to a protease inhibitor, such as 4-(2-aminoethy)-benzenesulfonyl fluoride. This type of protease inhibitor does not contain DNA contaminants, and thus can remain in the lysate during library preparation. The ability to inactivate the protease, e.g., proteinase K, thermolabile proteinase K, or the trypsin, helps to minimize damage to the enzymes used in downstream processes that take place at higher temperatures, e.g., subsequent library preparation processes that take place at higher temperatures. The inorganic salt free lysis buffer includes 1 unit to 5 units of the protease, i.e., a concentration ranging from about 5 units/mL to about 20 units/mL. In one example, the inorganic salt free lysis buffer includes 3.6 units of the thermolabile proteinase K. In another example, the inorganic salt free lysis buffer includes about 0.8 mg/mL of the proteinase K.

The inorganic salt free lysis buffer includes less than 2 mM of ethylenediaminetetraacetic acid (EDTA). For the DNA extraction from the whole blood sample, the EDTA may be used as a chelating agent to chelate metal ions that are present in enzymes, which work as co-factors to increase the enzyme’s catalytic activity. While EDTA readily deactivates DNase enzymes (which digest DNA), some of the metal ions chelated by EDTA may be desirable for the enzyme activity that is to take place in the subsequent library preparation technique. The relatively low EDTA concentration in the inorganic salt free lysis buffer enables the desirable metal ion chelation during the lysis procedure, without inhibiting the activity of these enzymes during library preparation. In one example, the concentration of EDTA ranges from about 0.5 mM to 1.5 mM. In another example, the concentration of EDTA is about 1 mM.

The inorganic salt free lysis buffer includes a chaotropic detergent. The chaotropic detergent may be useful in DNA extraction, but may also act as an inhibitor for enzymatic reactions occurring during library prep. One example of a chaotropic detergent is sodium dodecyl sulfate (SDS), which inhibits the transposase Tn5 used during the tagmentation reaction. Another example of a chaotropic detergent is guanidine hydrochloride. In an example, the chaotropic detergent is present in the inorganic salt free lysis buffer in an amount of about 0.1% w/v to about 0.5% w/v. As one specific example, the chaotropic detergent is present in the inorganic salt free lysis buffer in an amount of about 0.2% w/v.

The balance of the inorganic salt free lysis buffer is water, such as deionized water or another form of purified water.

In some examples, the inorganic salt free lysis buffer also includes suitable lysis buffer additives, such as a neutral buffer, e.g., Tris-HCI (pH 8), and a non-ionic detergent, e.g., TWEEN™ 20 (a polyoxyethylene sorbitol ester commercially available from Croda, Inc.). The neutral buffer may be present in a concentration ranging from about 25 mM to about 100 mM, and the non-ionic detergent may be present in an amount ranging from about 0.5% active w/v to about 0.75% active w/v. In one example, the inorganic salt free lysis buffer includes about 50 mM Tris-HCI (pH 8) and about 0.5% active w/v TWEEN™ 20.

As described herein, the lysis buffer is free of inorganic salt(s). While inorganic salt(s), including sodium chloride, may be beneficial during DNA extraction, it/they can also function as an inhibitor of one or more enzyme(s) used in library preparation processes. The exclusion of the inorganic salt contributes to the fact that the complexed crude lysate disclosed herein does not have to undergo purification before being used in library preparation.

The exposure of the whole blood sample to the inorganic salt free lysis buffer at the lysis temperature generates a crude lysate. The protease in the crude lysate is then inactivated using heat or an inhibitor. The inactivating agent used will depend upon the protease present.

In one example, the temperature of the crude lysate (and the contents contained therein) can be raised to about 55° C., which inactivates thermolabile proteinase K. In another example, the temperature of the crude lysate (and the contents contained therein) can be raised to about 80° C., which inactivates proteinase K. It is to be understood that the inactivation temperature may vary depending upon the protease that is used.

In another example, an inhibitor may be added to the crude lysate. As one specific example, when the inorganic salt free lysis buffer includes trypsin, inactivating the trypsin in the crude lysate involves exposing the crude lysate to the inhibitor, and the inhibitor is 4-(2-aminoethyl)-benzenesulfonyl fluoride. As another specific example, when the inorganic salt free lysis buffer includes proteinase K, inactivating the proteinase K in the crude lysate involves exposing the crude lysate to the inhibitor, and the inhibitor is tetrapeptidyl chloromethyl ketone (TPK). In an example, the ratio of inhibitor : serine protease ranges from 1:1: to 50:1. In one example, the inhibitor (e.g., TPK) that is introduced into the flow cell is present in an example of the tagmentation buffer in an amount ranging from about 0.04 mg/mL to about 0.1 mg/ mL. Any inhibitor that is selected should be free of DNA contaminants so that the crude lysate containing the inhibitor can be used in library preparation without first being exposed to purification.

After or as the protease, e.g., proteinase K, thermolabile proteinase K, or the trypsin, in the crude lysate is inactivated, the chelator of the chaotropic detergent is added to the crude lysate to generate a complexed crude lysate. The chelator that is used depends upon the chaotropic detergent that is used. As examples, the chelator of the chaotropic detergent is a cyclodextrin selected from the group consisting of alphacyclodextrin, betacyclodextrin, and methyl betacyclodextrin. As mentioned above, cyclodextrins are cup-shaped structures that have a hydrophilic exterior and a hydrophobic core. These compounds have the ability to form complexes with hydrophilic molecules, such as SDS or another chaotropic detergent in the lysis buffer. In particular, the long hydrophobic tail of SDS is drawn into the cup of the cyclodextrin, which effectively locks the SDS following lysis and prevents it from denaturing enzymes, including those used in downstream library preparation. As such, the complexed crude lysate can be used directly in library preparation without first being exposed to purification.

In some examples, the chelator is in dry form, the dry form of the chelator is part of an encapsulated complex including a coating material surrounding the dry form of the chelator. The chelator may be lyophilized or otherwise dried to form microspheres, which are then encapsulated by the coating material. In these examples, adding the chelator to the crude lysate involves adding the encapsulated complex to the crude lysate, then triggering release of the dry form of the chelator from the encapsulated complex, and rehydrating the dry form of the chelator. Rehydration of the chelator will precede any activity of the chelator. The release mechanism used will depend upon the type of coating material that surrounds the dry form of the chelator.

In one example, the coating material is a temperature sensitive wax; and triggering release of the dry form of the chelator involves heating the crude lysate to above a melting temperature of the temperature sensitive wax. Some examples of suitable temperature sensitive waxes include paraffin wax or soy wax, each of which melts at a temperature ranging from about 40° C. to about 50° C. Other examples of suitable temperature sensitive waxes include beeswax, microcrystalline polyethylene wax, or carnauba wax, each of which melts at a temperature ranging from about 60° C. to about 80° C. In these examples, the crude lysate is heated to the desired temperature to melt the wax and release the dry form of the chelator. The chelator is rehydrated and is able to sequester the chaotropic detergent to form the complexed crude lysate. Rehydration may be accomplished with water or another aqueous rehydration solution.

In another example, the coating material is a temperature sensitive polymer; and triggering release of the dry form of the chelator involves heating the crude lysate above solubilization temperature of the temperature sensitive polymer. Examples of suitable temperature sensitive polymers include poly(acrylamide-co-acrylonitrile), agarose, or gelatin, each of which solubilizes at a temperature greater than 30° C. Other examples of suitable temperature sensitive polymers include poly(N-isopropylacrylamide), methylcellulose, or poloxamer, each of which solubilizes at a temperature less than 30° C. In these examples, the crude lysate is heated to the desired temperature to solubilize the polymer and release the dry form of the chelator. The chelator is rehydrated (e.g., with water or an aqueous solution) and is able to sequester the chaotropic detergent to form the complexed crude lysate.

In still another example, the coating material is a pH sensitive polymer; and triggering release of the dry form of the chelator involves adjusting a pH of the crude lysate. Examples of suitable pH sensitive polymers include EUDRAGIT® L100 (methacrylic acid copolymer, type B from Evonik, Inc.) or KOLLICOAT® MAE-100 (methacrylic acid copolymer, type A from BASF Corp.), each of which is soluble at pH greater than 6. Other examples of suitable pH sensitive polymers include EUDRAGIT® RL/RS 100 (ammonio methacrylate from Evonik, Inc.) or carboxymethyl cellulose, each of which is soluble at pH greater than 8. Still other examples of suitable pH sensitive polymers include EUDRAGIT® E (amino dimethyl methacryate copolymer from Evonik, Inc.) or chitosan, each of which is soluble at pH less than 3. In these examples, the pH of the crude lysate is adjusted to be lower than the pH at which the polymer is sensitive (e.g., if solubility is pH X or lower) or to be higher than the pH at which the polymer is sensitive (e.g., if solubility is pH X or higher), and the polymer solubilizes. Solubilization of the polymer releases the dry form of the chelator. The chelator is rehydrated as described herein, and is able to sequester the chaotropic detergent to form the complexed crude lysate.

In yet a further example, the coating material is a time sensitive polymer; and triggering release of the dry form of the chelator involves allowing the encapsulated complex to incubate in the crude lysate for a predetermined amount of time. Examples of suitable time sensitive polymers include hydroxypropyl methylcellulose (a commercially available example of which is METHOCEL™ from DuPont), ethylcellulose (a commercially available example of which is ETHOCEL™ from DuPont), cellulose acetate (a commercially available example of which is OPADRY® CA from Colorcon), and poly(lactic-co-glycolic acid). In these examples, the crude lysate having the encapsulated complex therein is allowed to incubate for a predetermined time, which depends upon the type of polymer, the thickness of the polymer coating, and the temperature and makeup of the reaction taking place (e.g., pH). Over time, the polymer degrades and releases the dry form of the chelator. The chelator is rehydrated and is able to sequester the chaotropic detergent to form the complexed crude lysate.

The encapsulated complex may be formed by generating a dispersion of the coating material, spray coating the dispersion in a fluidized bed onto the dry form of the chelator, and allowing the complex to dry. Other suitable coating techniques, such a pan coating, spray drying, etc., may be used that effectively form a film of the coating material over the dry form of the chelator.

The chelator (whether encapsulated or not) may be mixed with a tagmentation buffer, such as a combination of Tris-acetate, magnesium acetate, and water. The use of the tagmentation buffer may be particularly desirable when the complexed crude lysate is to be used in a tagmentation library preparation process. It is to be understood that the tagmentation buffer may initiate the release of the dry form of the chelator, and thus incorporation of the encapsulated chelator and the tagmentation buffer may depend upon the coating of the encapsulated chelator, its dissolution characteristics, and the desired timing for achieving complexation.

In some examples, the inhibitor of the protease and the chelator (whether encapsulated or not) of the chaotropic agent may also be mixed with the tagmentation buffer. In this example, inactivation of the protease and generation of the complexed crude lysate may take place at the same time.

When incorporated into the tagmentation buffer, the amount of the chelator may range from about 2% w/v to about 5% w/v.

The complexed crude lysate may then be exposed to a library preparation technique. The extracted DNA sample in the complexed crude lysate may be exposed to tagmentation, or any other library preparation technique that fragments the longer piece(s) of genetic material and incorporates the desired adapters to the ends of the fragments.

In one example, the complexed crude lysate disclosed herein may be introduced into any example of the flow cell disclosed herein, where on flow cell tagmentation may be performed as described herein. As such, some examples of the method involve introducing the complexed crude lysate to a flow cell including transposome complexes 10, 10′ and primers 36, 36′ immobilized to a surface within the flow cell. In this example, the extracted deoxyribonucleic acids (in the complexed crude lysate) are exposed to tagmentation by: introducing a tagmentation buffer to the flow cell as the complexed crude lysate is introduced, and bringing the flow cell to a temperature of 30° C. or higher (as described herein in reference to FIG. 1C). In this example, the transposase 14, 14′ may be removed and amplification may be performed by: introducing a washing solution into the flow cell; heating the flow cell, containing the washing solution, to about 60° C.; and then introducing an extension amplification mix into the flow cell (as described in reference to FIG. 1D).

In one example, the DNA sample preparation method disclosed herein, i.e., the exposing, the inactivating, and the adding, are performed in a lysis tube or chamber off board the flow cell.

When the lysis tube is used, the complexed crude lysate may be dumped from the lysis tube into a sample input port of the sequencing instrument. Then, the fluid delivery system of the sequencing instrument delivers the complexed crude lysate to the flow cell for tagmentation and subsequent processing.

The chamber may be a DNA sample preparation chamber that is a component of the sequencing instrument. Thus, when the chamber is used, the complexed crude lysate may be transported from the chamber to the flow cell for tagmentation and subsequent processing.

Another example sample preparation method includes: at a temperature ranging from about 18° C. to about 25° C., exposing a cell sample to zinc oxide nanomaterials, thereby initiating detergent free extraction of deoxyribonucleic acids or ribonucleic acids from the cell sample and generating a crude lysate; and exposing the extracted deoxyribonucleic acids or ribonucleic acids in the crude lysate to tagmentation. In an example, the zinc oxide nanomaterials are added to the cell sample. In another example, the zinc oxide nanomaterials are dispersed in water and the dispersion is added to the cell sample. In the latter example, the cell sample may be in the form of a cell-pellet that is added to a zinc oxide nanomaterial dispersion.

The cell sample may be from any eukaryotic or prokaryotic cells, as well as from fungi cells. Some examples of the cell samples include whole blood, bone marrow aspirate, serum, plasma, tissue, blood spots, saliva, and other bodily fluids.

The zinc oxide nanomaterials may be in the form of nanoparticles, nanotubes, nanowires, or the like. The nanoparticles may have an average particle size ranging from about 1 nm to about 1000 nm. The nanotubes or nanowires may have an average diameter ranging from about 1 nm to about 1000 nm. In one example, the zinc oxide nanoparticles have an average particle size ranging from about 10 nm to about 950 nm. In one example, the zinc oxide nanoparticles have an average particle size of about 300 nm. The average particle size may represent the mean value for a distribution of particles, which may be associated with the basis of the distribution calculation (number, surface, or volume). As such, in some examples, the average particle size may represent a volume mean diameter, a number mean diameter, or a surface mean diameter. The average particle size may be determined using a particle size analyzer, or using X-ray diffraction (XRD), scanning electron microscopy (SEM), transmission electron microscopy (TEM), etc. The use of the zinc oxide nanomaterials enables this DNA or RNA extraction to be detergent free.

At the outset of this example method, the cell sample and the zinc oxide nanomaterials are mixed together at a 1.5:1 to 1:1.5 volume:volume ratio or volume:weight ratio. In one example, the cell sample and the zinc oxide nanomaterials are mixed together at a 1:1 volume:volume ratio or volume:weight ratio. In one example of the method, the volume of each of the cell sample and the zinc oxide nanoparticles ranges from about 20 µL to about 100 µL. The cell sample and zinc oxide nanomaterials are mixed together and are allowed to incubate for a time ranging from about 2 minutes to about 30 minutes. During incubation, the cells are ruptured, which leads to DNA or RNA extraction. The mechanism by which rupture occurs may be chemical, where excessive Zn²⁺ released from the zinc oxide nanomaterials adsorbs on the cell membrane surface and then penetrates the cell wall causing rupture, and/or biological, where the zinc oxide nanomaterials trigger a reactive oxygen species (ROS) reaction that leads to apoptotic cell death and collapse of the cellular structure.

The DNA (e.g., gDNA) or RNA extraction using the zinc oxide nanomaterials can take place at room temperature, e.g., from about 18° C. to about 25° C., and thus additional heating and heating equipment is not utilized.

In one example, the crude lysate is generated in a sample collection device (e.g., a lysis tube, MITRA® tips, dried blood spot cards, etc.), and the zinc oxide nanomaterials are dried on a surface of the sample collection device. In this example, the addition of the cell sample activates lysis.

In some examples, the zinc oxide nanomaterials are mixed with a protease. The protease (e.g., serine protease, such as thermolabile proteinase K) may be included to digest proteins that may deleteriously affect the DNA or RNA during extraction and to protect the extracted DNA or RNA from nucleases. The protease may be added in an amount ranging from 1 unit to 5 units, i.e., a concentration ranging from about 5 units/mL to about 20 units/mL. The temperature at which the protease is active will depend upon the protease that is used. Thermolabile proteinase K is active at low temperatures ranging from about 20° C. to about 40° C., and may be fully inactivated at about 55° C. The ability to inactivate the protease, e.g., thermolabile proteinase K, helps to minimize damage to the enzymes used in downstream processes that take place at higher temperatures, e.g., subsequent library preparation processes that take place at higher temperatures.

When the cell sample is a blood sample, hemoglobin interference may take place. To avoid hemoglobin interference, the zinc oxide nanomaterials may be mixed with hemoglobin capture agents, such as HemogloBind™ (from Biotech Support Group) and EasySep™ Direct RapidSpheres™ (from StemCell Technologies). Each of these hemoglobin capture agents may be used as directed by the manufacturer.

As noted herein, the zinc oxide nanomaterial can induce the generation of a reactive oxygen species. This species can potentially damage the extracted DNA or RNA, and thus it may be desirable to add an ROS-scavenger to reduce the likelihood of extracted DNA or RNA damage. The amount of ROS-scavenger may depend on how much of the zinc is present. In an example, the ROS-scavenger is added in an amount ranging from about 2 mM to about 50 mM.

The exposure of the cell sample to the zinc oxide nanomaterials generates a crude lysate. The crude lysate may then be exposed to a library preparation technique.

In some examples of this method, the zinc oxide nanomaterials are combined with the protease; and prior to exposing the extracted deoxyribonucleic acids in the crude lysate to tagmentation, the method further comprises inactivating the protease using heat. For example, the temperature of the crude lysate (and the contents contained therein) can be raised to about 55° C., which inactivates thermolabile proteinase K. It is to be understood that the inactivation temperature may vary depending upon the protease that is used.

For library preparation, the extracted DNA or RNA sample in the crude lysate may be exposed to tagmentation, or to any other library preparation technique that fragments the longer piece(s) of genetic material and incorporates the desired adapters to the ends of the fragments.

In one example, the crude lysate generated via this example of the method may be introduced into any example of the flow cell disclosed herein, where on flow cell tagmentation may be performed as described herein. As such, some examples of the method involve introducing the crude lysate to a flow cell including transposome complexes 10, 10′ and primers 36, 36′ immobilized to a surface within the flow cell. In this example, the extracted deoxyribonucleic acids or ribonucleic acids (in the crude lysate) are exposed to tagmentation by: introducing a tagmentation buffer to the flow cell as the crude lysate is introduced, and bringing the flow cell to a temperature of 30° C. or higher (as described herein in reference to FIG. 1B). In this example, the tagmentation buffer includes water, an optional co-solvent (e.g., dimethylformamide), and a buffer salt (e.g., tris acetate salt, pH 7.6). Some examples of this tagmentation buffer also include the metal co-factor (e.g., magnesium acetate). Because the crude lysate includes Zn²⁺, this example of the tagmentation buffer may alternatively be free of the metal co-factor (e.g., magnesium acetate) for the transposase of the transposome complex. In this particular example, the zinc oxide materials may be used in excess. After tagmentation, the transposase may be removed and amplification performed by heating the flow cell to about 60° C.; flowing a washing solution through the flow cell; and introducing an amplification mix into the flow cell (as described in reference to FIG. 1C).

In one example, the DNA sample preparation method utilizing the zinc oxide nanomaterials takes place in a chamber off board the flow cell. The chamber may be a DNA sample preparation chamber that is a component of the sequencing instrument. Thus, when the chamber is used, the crude lysate may be transported from the chamber to the flow cell for on flow cell tagmentation and subsequent processing.

In another example, the DNA sample preparation method utilizing the zinc oxide nanomaterials and the tagmentation are performed in a lysis tube. When the lysis tube is used, bead based tagmentation or solution based tagmentation may be used.

For bead based tagmentation, a solid support (i.e., the bead) has surface bound transposome complexes (similar to 10, 10′) and surface bound primers (similar to 36, 36′). Tagmentation takes place on the surface of the solid support, so that the resulting DNA fragments are attached to the surface. A tagmentation buffer and the solid support including the surface bound transposome complexes and the surface bound primers are introduced into the lysis tube, where tagmentation, ligation, and extension take place (e.g., as described in FIG. 1A through FIG. 1C). In this example, the tagmentation buffer includes water, an optional co-solvent (e.g., dimethylformamide), a buffer salt (e.g., tris acetate salt, pH 7.6), a recombinase, a polymerase, accessory proteins, and an optional metal co-factor.

For solution based tagmentation, tagmentation takes place in solution with free floating transposomes and primers. A tagmentation buffer is introduced into the lysis tube, where tagmentation, ligation, and extension take place (e.g., as described in FIG. 1A through FIG. 1C). In this example, the tagmentation buffer includes transposomes, primers, water, an optional co-solvent (e.g., dimethylformamide), a buffer salt (e.g., tris acetate salt, pH 7.6), a recombinase, a polymerase, accessory proteins, and an optional metal co-factor.

### Dried Reagents

In any of the examples set forth herein, one or more of the reagents for flow cell preparation and/or for sample preparation may be dried and stored in a cartridge before being introduced onto the flow cell. Drying may be performed using lyophilization, spray drying, vacuum drying, or microwave drying. Each of the dried reagents can be reconstituted in water before being used to prepare the flow cell and/or the sample.

Lyophilization is a freeze drying process that removes water from the reagent after it is frozen and placed under a vacuum. Lyophilization may be performed at a temperature ranging from about -30° C. to about -50° C., and at a pressure ranging from about 30 mPa to about 90 mPa. This process generates solid microspheres of the reagent components, and thus is more condense (than the corresponding liquid) and more stable for storage and shipping.

In one example, a transposome buffer is dried. The transposome buffer is used to introduce the transposome complexes 10, 10′, 10A, 10B, 10C, 10D to the flow cell. The transposome buffer contains any example of the transposomes 10, 10′, 10A, 10B, 10C, 10D in a buffer solution. Pre-drying, the transposome buffer solution may include a buffer agent (e.g., Tris), a salt (e.g., sodium chloride, sodium citrate, etc.), a surfactant (e.g., TWEEN polysorbates), in some instances a lyophilization excipient (e.g., Trehalose), and water.

In another example, streptavidin is dried. In accordance with some of the methods disclosed herein, the streptavidin is used to attach transposome complexes 10, 10′, 10A, 10B, 10C, 10D to the biotinylated polymeric hydrogel. Pre-drying, streptavidin may be contained in a buffer containing the buffer agent (e.g., Tris), the salt (e.g., sodium chloride, sodium citrate, etc.), the surfactant (e.g., TWEEN polysorbates), in some instances the lyophilization excipient (e.g., Trehalose), and water.

In still another example, the inorganic salt free lysis buffer disclosed herein may be dried. Any example of the inorganic salt free lysis buffer may be dried. When lyophilization is used, any example of the inorganic salt free lysis buffer disclosed herein is mixed with the lyophilization excipient before lyophilization is performed.

In yet another example, the tagmentation buffer disclosed herein may be dried. Any example of the tagmentation buffer may be dried. When lyophilization is used, any example of the tagmentation buffer disclosed herein is mixed with the lyophilization excipient before lyophilization is performed.

In any of the reagents that are to be lyophilized, the lyophilization excipient may be present at a concentration ranging from about 200 mM to about 700 mM.

### Sequencing Instrument

Any example of the flow cell may be introduced into a sequencing instrument which includes a flow cell receptacle; a fluidic control system including delivery fluidics to respectively deliver the DNA sample (e.g., the complexed crude lysate), wash solutions, extension amplification mixtures, and other reagents to the flow cell positioned in the flow cell receptacle; an illumination system positioned to illuminate the flow cell positioned in the flow cell receptacle; a detection system positioned to capture an image of the flow cell positioned in the flow cell receptacle; and a controller in operative communication with the fluidic control system, the illumination system, and the detection system. When in position, the flow cell is in fluid communication with the fluidic control system (e.g., pumps, valves, and the like) and is in optical communication with the illumination system and the detection system.

The sequencing instrument may also include a DNA sample preparation chamber, where, e.g., a whole blood sample (e.g., the complexed crude lysate) or other like sample is introduced and exposed to lysis or another technique before it is introduced into the flow cell for tagmentation, amplification, and sequencing. The DNA sample preparation chamber may have a heater in a position to heat the sample introduced thereto to a desirable temperature. A fluid delivery line may connect the DNA sample preparation chamber and the flow cell, and a valve may be positioned at the input port of the flow cell to open and close the flow cell.

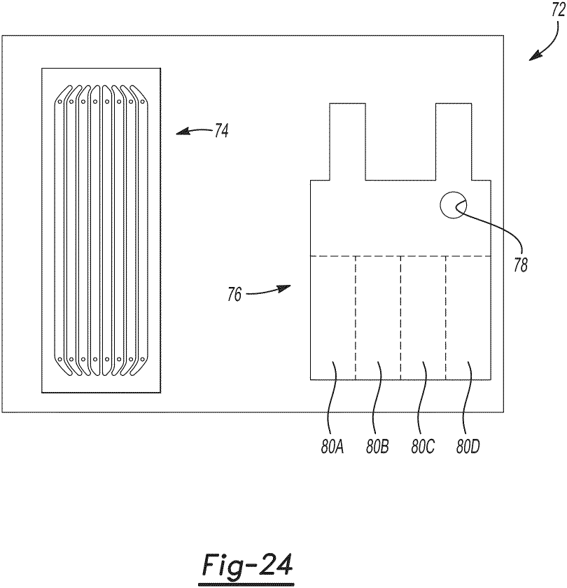

The fluidic system of the sequencing instrument may also include a receptacle for receiving a reagent cartridge (an example of which is shown schematically in FIG. 24 at reference numeral 76). The reagent cartridge 76 includes a fluid input port 78 that can receive a sample (e.g., a blood sample, bone marrow aspirate, etc.); and fluid receptacles 80A, 80B, 80C, 80D that are isolated from one another for storing different reagents (in liquid or dried form). While not shown in FIG. 24, each fluid receptacle 80A, 80B, 80C, 80D is selectively fluidly connected to a fluid line that can deliver the reagent contained therein to a mixing chamber (not shown) for reconstitution before being delivered to a flow cell (reference numeral 74 of FIG. 24), or to the flow cell 74 when both the reagent cartridge 76 and the flow cell 72 are inserted into their respective positions within the sequencing instrument.

### Kits

Any of the flow cells and reagents disclosed herein may be included in a tagmentation kit. An example kit is shown in FIG. 24. In an example, the kit 72 includes a flow cell 74 and a reagent cartridge 76. The flow cell 74 may be any of the examples described herein.

In some example kits 72, the flow cell 74 includes the immobilized transposome complexes 10, 10′ or 10A, 10B described herein, and the kit 72 further includes the tagmentation buffer, the inorganic salt free lysis buffer, and/or an inhibitor of the chaotropic agent. In one example, the kit 72 includes a combined tagmentation buffer and inhibitor of the chaotropic agent. Each of these fluids may be included in a separate receptacle 80A, 80B, 80C, 80D of the reagent cartridge 76.

In other example kits 72, the flow cell 74 includes the different chemistry for assembling the transposome complex 10C and for attaching the transposome complex 10D (as described herein), and the kit 72 further includes a transposome buffer with the components for forming the transposome complex 10C, a second transposome buffer with the transposome complexes 10D, and a tagmentation buffer. Each of these fluids may be included in a separate receptacle 80A, 80B, 80C of the reagent cartridge 76. This example kit 72 may also include any of the lysis buffers, inhibitors, or other fluids disclosed herein.

In still other example kits 72, the flow cell 74 does not initially include any of the transposomes disclosed herein.

In one example of this type of kit 72, the reagent cartridge 76 is loaded with reagent(s) to introduce the transposomes 10, 10′. For example, the flow cell 74 may include the substrate 40 having depressions 12 separated by interstitial regions 38, first and second primers 36, 36′ immobilized within each of the depressions 12, and the biotin-containing linker 42 immobilized within each of the depressions 12 (e.g., attached to the polymeric hydrogel 24); and the reagent cartridge 76 may include the fluid input port 78, and receptacles 80A, 80B, 80C, 80D respectively containing: the transposome buffer including first transposome complexes 10, each having a first amplification domain 26 and a 5′ biotinylated end 22, and second transposome complexes 10′, each having a second amplification domain 26′ and a 5′ biotinylated end 22′; and a tagmentation buffer.

In another example of this type of kit 72, the reagent cartridge 76 is loaded with reagent(s) to prepare the flow cell surface for transposome introduction and to introduce the transposomes 10, 10′. For example, the flow cell 74 may include the substrate 40 having depressions 12 separated by interstitial regions 38, and first and second primers 36, 36′ immobilized within each of the depressions 12; and the reagent cartridge 76 may include the fluid input port 78, and receptacles 80A, 80B, 80C, 80D respectively containing: a biotin-containing linker 42; and a transposome buffer including first transposome complexes 10, each having a first amplification domain 26 and a 5′ biotinylated end 22, and second transposome complexes 10′, each having a second amplification domain 26′ and a 5′ biotinylated end 22′.

In either of these example kits 72, when the sample to be introduced into the reagent cartridge 76 is selected from the group consisting of whole blood, a tissue sample, blood spots, and saliva, the reagent cartridge 76 may further include any example of the inorganic salt free lysis buffer in one of the receptacles 80A, 80B, 80C, 80D. In these examples, i) the inorganic salt free lysis buffer is a liquid; and the inorganic salt free lysis buffer further includes water; or ii) the inorganic salt free buffer is a dried solid; and the reagent cartridge 76 further includes a reconstitution fluid (e.g., water) in another one of the receptacles 80A, 80B, 80C, 80D. Also in these examples, the reagent cartridge 76 may further include an inhibitor of the chaotropic detergent in another one of the receptacles 80A, 80B, 80C, 80D.

### Methods With One Transposome Type at the Outset

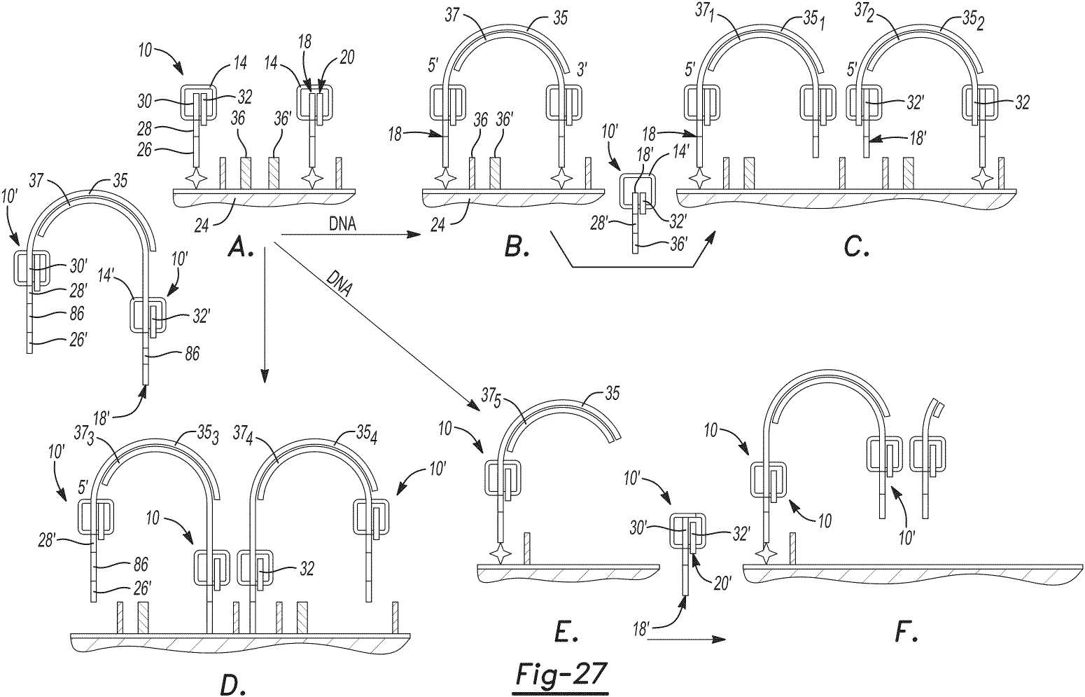

Several of the examples disclosed herein utilize two different transposome complexes 10, 10′ or 10A, 10B, etc. The example methods shown in FIG. 27 begin with one type of transposome complex 10 or 10′ (not both) in the depressions 12 of the flow cell, and the other type of the transposome complex 10′ or 10 is subsequently introduced. As such, the transposome complexes 10, 10′ are loaded sequentially. One example of the methods in FIG. 27 moves from A to B to C. Another example of the methods moves from A to D. Still another example of the methods moves from A to E to F.

The example method in FIG. 27 that includes A, B, and C will now be described. At A in FIG. 27, a portion of the bottom surface of the depression 12 is depicted. The depression 12 includes the polymeric hydrogel 24, the one type of transposome complex 10 immobilized to the polymeric hydrogel 24, and the primers 36, 36′ immobilized to the polymeric hydrogel 24. Immobilization may be accomplished using any of the functional groups described herein. In one example, the polymeric hydrogel 24 includes azide or tetrazine surface groups, and the 5′ end groups of the complexes 10 and of the primers 36, 36′ each include a terminal alkyne (e.g., hexynyl) or an internal alkyne, where the alkyne is part of a cyclic compound (e.g., bicyclo[6.1.0]nonyne (BCN)).

As described herein, the one (first) type of transposome complex 10 includes the transposon end 16 with a portion 30 of the transferred strand 18 hybridized to the non-transferred strand 20, 32, and the transferred strand 18 includes the first amplification domain 26.

This example method involves generating double stranded DNA fragments 35, 37 on the flow cell surface using a plurality of the first type of transposome complexes 10 attached to the surface, wherein the double stranded DNA fragments 35, 37 are respectively attached at their 5′ ends to 3′ ends of the first transferred strands 18 (see B in FIG. 27); and introducing a plurality of a second type of transposome complexes 10′ to the flow cell, thereby tagmenting the double stranded DNA fragments 35, 37 to generate new double stranded DNA fragments 35₁, 37₁ and 35₂, 37₂ respectively attached at their 5′ ends to 3′ ends of the second transferred strands 18′ (see C in FIG. 27).

In this example, generating the double stranded DNA fragments 35, 37 includes introducing the DNA sample and the tagmentation buffer to the flow cell; and performing tagmentation of the DNA sample using the first type of transposome complex 10. The specifics of tagmentation are described herein in reference to FIG. 1C. After tagmentation, the flow cell may be flushed with an example of the washing solution disclosed herein.

The second type of transposome complexes 10′ are then introduced into the flow cell. The already fragmented DNA fragments 35, 37 are again fragmented, and the 5′ ends of newly fragmented strands 37₁, 35₂ are ligated to respective 3′ ends of the transferred strands 18′ of the transposome complexes 10′. The second tagmentation is shown at C in FIG. 27. In this example, the second occurrence of tagmentation may take place at a temperature at or above 30° C. In one example, the temperature may range from 30° C. to about 55° C.

The second tagmentation results in fragmented new DNA fragments 35₁, 37₁ and 35₂, 37₂. The 5′ ends of 35₁, 37₁ are respectively attached to the transferred strands 18, 18′ of the different transposome complexes 10, 10′ and the 5′ ends of 35₂, 37₂ are respectively attached to the transferred strands 18′, 18 of the different transposome complexes 10′, 10. As depicted at C, the 3′ ends of the newly fragmented strands 35₁, 37₂ are not ligated to the 5′ ends of the non-transferred strands 32′ (from the complexes 10′). As such, a gap exists between the 3′ ends of the DNA fragment strand 35₁, 37₂ and the 5′ end of the corresponding non-transferred strands 32′.

The method then includes removing the transposase enzymes 14, 14′ of each of the first and second type of transposome complexes 10, 10′; and performing an extension reaction to add additional adapters to the new double stranded DNA fragments.

The transposase enzymes 14, 14′ may be removed using SDS or another chaotropic detergent or heat as described herein. When SDS or another chaotropic detergent has been used for transposase removal, the washing solution may be flushed through the flow channel prior to initiating the extension reaction. This removes the chaotropic detergent, which may interfere with downstream enzyme activity. Alternatively, a chelator of the chaotropic agent may be introduced as described herein.

To initiate the extension reaction, an extension amplification mix is into the flow cell. The example extension amplification mix includes a recombinase, a polymerase, and accessory proteins. The flow cell may be at about 38° C. when the extension amplification mix is introduced.

Prior to the extension reaction, the non-transferred strands 32, 32′ may first be dehybridized using heat.

Additional sequences (adapters) are added to the 3′ ends of the partially adapted fragments by an extension reaction using exclusion amplification reagents (e.g., the ExAMP reagents available from Illumina Inc.). The extension reaction involves the addition of nucleotides in a template dependent fashion from the 3′ ends of the DNA fragments 35₁, 37₁ and 35₂, 37₂ using the respective transferred strands 18, 18′ as the template. The sequences resulting from the extension reaction render the partially adapted DNA fragments 35₁, 37₁ and 35₂, 37₂ fully adapted and ready for further amplification and cluster generation. Amplification and cluster generation may be performed as described in reference to FIG. 1C.

In this example, the density of the transposome complexes 10 introduced to the flow cell surface is controlled at a low level (e.g., < 0.05 µM) so that (desirably) one transposome complex 10 is loaded into one depression 12.

The example method in FIG. 27 that includes A and D will now be described. In this example, one tagmentation process is performed off of the flow cell, and a second tagmentation process is performed on the flow cell. This method includes tagmentating a DNA sample in solution using a plurality of transposome complexes 10′, each including a transposon end 16′ with a portion of the transferred strand 18′ hybridized to the non-transferred strand 20′, wherein the transferred strand 18′ includes an amplification domain 26′ and an index sequence 86, thereby generating double stranded DNA fragments 35, 37 respectively attached at their 5′ ends to 3′ ends of the transferred strands 18′; and introducing the solution, including the double stranded DNA fragments 35, 37, to a flow cell including a plurality of a another type of transposome complexes 10 immobilized to a surface thereof, the other type of transposome complexes 10 including the transposon end 16 with the portion of the transferred strand 18 hybridized to the non-transferred strand 20, wherein the transferred strand 18 includes the amplification domain 26 and is free of an index sequence, thereby tagmenting the double stranded DNA fragments 35, 37 to generate new double stranded DNA fragments 35₃, 37₃, 35₄, 37₄ respectively attached at their 5′ ends to 3′ ends of the transferred strands 18.

The tagmentation process that is performed off of the flow cell is depicted between A and D in FIG. 27. In this example, the DNA sample and the transposome complexes 10′ are added to a tagmentation buffer in a reaction vessel. In solution, the DNA sample is tagmented. While the transposome complexes 10′ as shown, it is to be understood that the complexes 10 could be used for the in solution tagmentation. Additionally, the complexes 10 attached to the flow cell surface could also include the index/barcode sequence.

The tagmented double stranded DNA fragments 35, 37, with the transposome complex 10′ attached thereto are then introduced into the flow cell shown in A. On the flow cell, the already fragmented DNA fragments 35, 37 are again fragmented, and the 5′ ends of newly fragmented strands 37₃, 35₄ are tagmented to respective 3′ ends of the transferred strands 18 of the surface bound transposome complexes 10. The second tagmentation is shown at D in FIG. 27. In this example, the second occurrence of fragmentation and ligation may take place at a temperature at or above 30° C. In one example, the temperature may range from 30° C. to about 55° C.

The second tagmentation results in fragmented new DNA fragments 35₃, 37₃ and 35₄, 37₄. The 5′ ends of 35₃, 37 are respectively attached to the transferred strands 18′, 18 of the different transposome complexes 10′, 10 and the 5′ ends of 35₄, 37₄ are respectively attached to the transferred strands 18, 18′ of the different transposome complexes 10, 10′. As depicted at E, the 3′ ends of the newly fragmented strands 35₃, 37₄ are not ligated to the 5′ ends of the non-transferred strands 32 (from the complexes 10). As such, a gap exists between the 3′ ends of the DNA fragment strand 35₃, 37₄ and the 5′ end of the corresponding non-transferred strands 32.

The method then includes removing the transposase enzymes 14, 14′ of each of the first and second type of transposome complexes 10, 10′; and performing an extension reaction to add additional adapters to the new double stranded DNA fragments.

Transposase enzyme 14, 14′ removal and the extension reaction may be performed as described herein. The sequences resulting from the extension reaction render the partially adapted DNA fragments 35₃, 37₃ and 35₄, 37₄ fully adapted and ready for further amplification and cluster generation. Amplification and cluster generation may be performed as described in reference to FIG. 1C.

The example method in FIG. 27 that includes A, E and F will now be described. This method includes introducing one transposome complex 10 of a single type into the depression 12 of a flow cell, the transposome complex 10 including the transposon end 16 with the portion 30 of the transferred strand 18 hybridized to the non-transferred strand 20, wherein the transferred strand 18 includes a first amplification domain 26; introducing a DNA sample into the flow cell, whereby the one transposome complex tagments the DNA sample to generate a double stranded DNA fragment 35, 37₅ having a first strand 35 attached at its 5′ end to a 3′ end of the transferred strand 18 and unattached at its 3′ end, and having a second strand 37₅ unattached at both its 3′ and 5′ ends (see E in FIG. 27); and introducing a second type of transposome complex 10′ to the flow cell, the second type of transposome complex 10′ including the second transposon end 16′ with the portion 30′ of the second transferred strand 18′ hybridized to the second non-transferred strand 20′, wherein the second transferred strand 18′ includes a second amplification domain 26′, whereby the 5′ end of the second strand 37₅ of the double stranded DNA fragment attaches to the second transferred strand 18′.

In this example, the density of the transposome complexes 10 introduced to the flow cell surface is controlled at a low level (e.g., < 0.05 µM) so that (desirably) one transposome complex 10 is loaded into one depression 12. Thus, during tagmentation, the DNA sample would be loosely attach so that one end is attached to the transposome complex 10 in the depression 12 and the other end is suspended in the bulk tagmentation fluid (as shown at E).

The flow cell may be washed with the washing solution, and then the other transposome complexes 10′ are introduced into the flow cell. The 3′ end of the transferred strand 18′ of the newly introduced transposome complexes 10′ will attach to the 5′ end of the second strand 37₅. This renders the second strand 37₅ partially adapted.

The method then includes removing the transposase enzymes 14, 14′ of each of the first and second type of transposome complexes 10, 10′; and performing an extension reaction to add additional adapters to the new double stranded DNA fragments.

Transposase enzyme 14, 14′ removal and the extension reaction may be performed as described herein. The sequences resulting from the extension reaction render the partially adapted DNA fragments 35, 37₅ fully adapted and ready for further amplification and cluster generation. Amplification and cluster generation may be performed as described in reference to FIG. 1C.

The example shown in FIG. 27 at A, E, and F may take placed in combination with the method shown at A, B, and C, and the tagmentation(s) shown in E and F may occur at or near the end of a DNA sample (e.g., that is not long enough to reach the next immobilized transposome complex 10.

To further illustrate the present disclosure, examples are given herein. It is to be understood that these examples are provided for illustrative purposes and are not to be construed as limiting the scope of the present disclosure.

### NON-LIMITING WORKING EXAMPLES

### Example 1

Flow cells including PAZAM and grafted P5 and P7 primers in the depressions were used.

P5 primers having a 3′ mosaic end and a 5′ BCN linker were mixed with an equivalent amount of a complementary mosaic end (including a 5′ phosphate). The mixture was incubated for about 2 minutes at about 95° C. and then was cooled to 10° C. at a rate of -0.1° C./s. This formed transposon P5. The transposon P5 was mixed with double the amount of Tn5 transposase in a buffer solution, and the mixture was allowed to incubate at 37° C. for 12-24 hours. This formed BCN-P5 transposomes. The same procedure was performed with P7 primers having a 3′ mosaic end to form BCN-P7 transposomes.

The BCN-P5 and BCN-P7 transposome complexes were grafted to the PAZAM in two different flow cells. The BCN-P5 and BCN-P7 transposome complexes were diluted in a sodium sulfate grafting buffer to form a transposome mixture. The flow cells were washed with deionized water, flushed with a sodium sulfate solution, and heated up to 37° C. The transposome mixture was added to each flow cell and incubated for about 2 hours. After the desired incubation period, the flow cells were flushed with a buffer, and were cooled to room temperature.

A DNA sample was prepared in water so that the total input was between 300 ng and 800 ng. Additional water was added along with a tagmentation buffer.

As a control, the DNA sample (about 150 µL) was introduced into one of the flow cells and tagmentation was allowed to occur. The control was performed twice.

In an example, the DNA sample (about 150 µL) was introduced with 5 mM spermidine and tagmentation was allowed to occur. The example was performed twice.

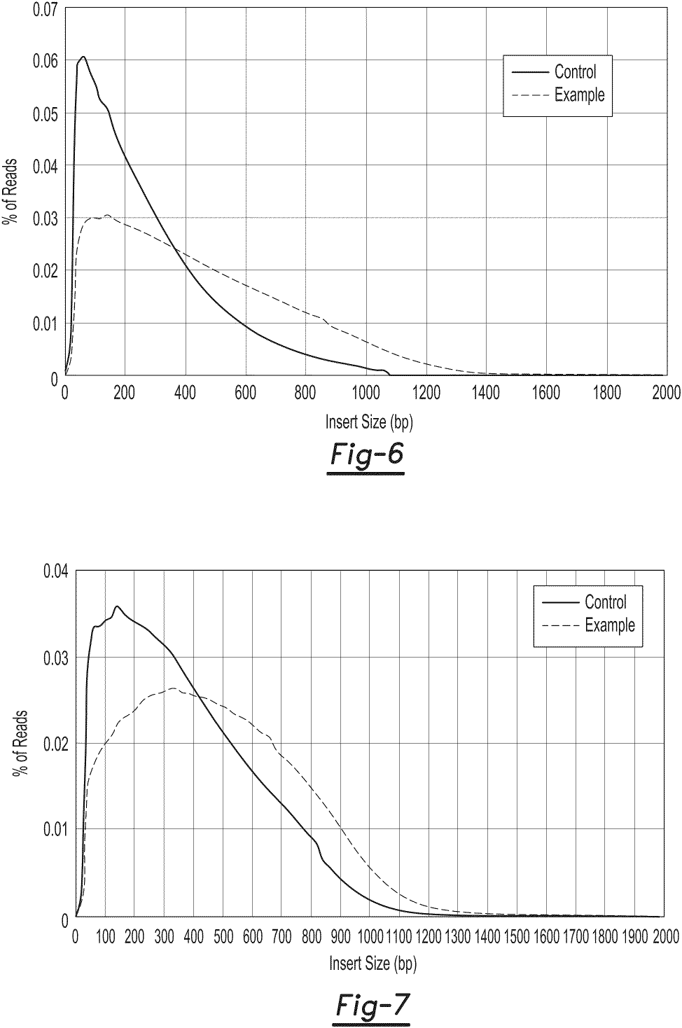

The insert size of the tagmented control DNA samples and the tagmented example DNA samples were determined by sequencing and aligning the data using Illumina’s Dynamic Read Analysis for GENomics (DRAGEN) software. The results are shown in FIG. 6. As depicted, the presence of spermidine increased the insert size.

### Example 2

Flow cells including PAZAM and grafted P5 and P7 primers in the depressions were used.

As a control, the BCN-P5 and BCN-P7 transposome complexes from Example 1 were used. They were introduced into the control flow cell in the same manner described in Example 1.

As an example, BCN-P5 and BCN-P7 transposome complexes without 5′ phosphates on the non-transferred strands were prepared as described in Example 1, except that the complementary mosaic ends did not include a 5′ phosphate. The BCN-P5 and BCN-P7 transposome complexes without 5′ phosphates on the non-transferred strands were introduced into the example flow cell in the same manner described in Example 1.

The DNA sample was prepared as described in Example 1 and was introduced into each of the control flow cell and the example flow cell and tagmentation was allowed to occur.

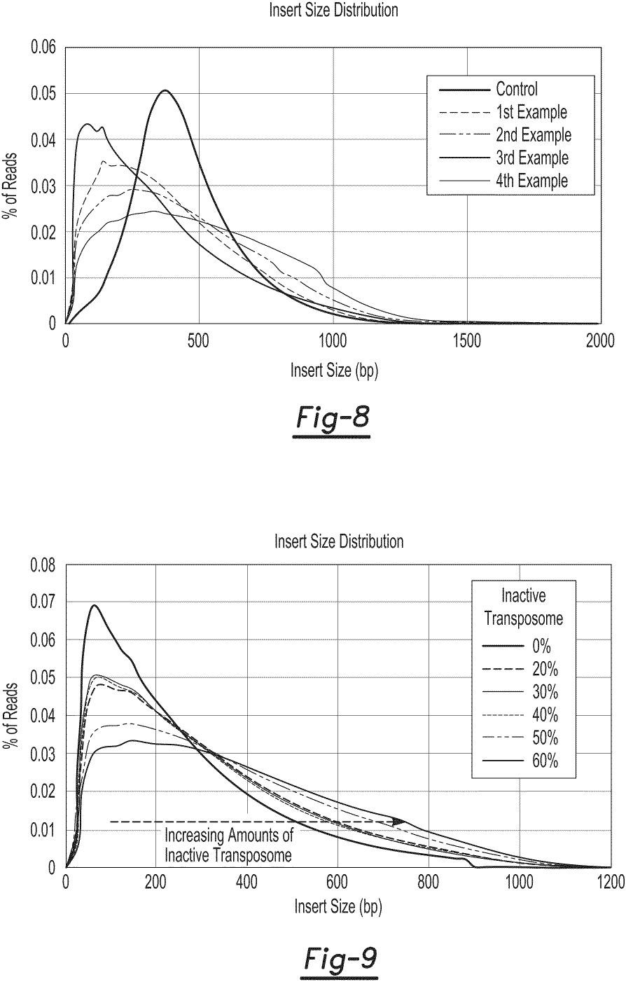

The insert size of the tagmented DNA sample in the control flow cell and the tagmented DNA sample in the example flow cell were determined by sequencing and aligning the data using Illumina’s Dynamic Read Analysis for GENomics (DRAGEN) software. The results are shown in FIG. 7. As depicted, the exclusion of the 5′ phosphate increased the insert size. These results indicate that modifications to the transposon can affect the insert size.

### Example 3

Flow cells including PAZAM and grafted P5 and P7 primers in the depressions were used.

As a first example, the BCN-P5 and BCN-P7 transposome complexes (with 5′ phosphate non-transferred strands) from Example 1 were used. They were introduced into the first example flow cell in the same manner described in Example 1.

As a second example, BCN-P5 and BCN-P7 transposome complexes without a 5′ phosphate on the non-transferred strands and with a 3′ ALEXA FLUOR® 647 dye on the non-transferred strands were prepared as described in Example 1, except that the complementary mosaic ends did not include the 5′ phosphate and did include the 3′ ALEXA FLUOR® 647 dye. The BCN-P5 and BCN-P7 transposome complexes without 5′ phosphates and with 3′ ALEXA FLUOR® 647 dyes on the non-transferred strands were introduced into the second example flow cell in the same manner described in Example 1.

As a third example, BCN-P5 and BCN-P7 transposome complexes with a 5′ phosphate on the non-transferred strands and with a 3′ ALEXA FLUOR® 647 dye on the non-transferred strands were prepared as described in Example 1, except that the complementary mosaic ends included the 3′ ALEXA FLUOR® 647 dye. The BCN-P5 and BCN-P7 transposome complexes with 5′ phosphates and with 3′ ALEXA FLUOR® 647 dyes on the non-transferred strands were introduced into the third example flow cell in the same manner described in Example 1.

As a fourth example, BCN-P5 and BCN-P7 transposome complexes without a 5′ phosphate on the non-transferred strands were prepared as described in Example 1, except that the complementary mosaic ends did not include the 5′ phosphate. The BCN-P5 and BCN-P7 transposome complexes without 5′ phosphates on the non-transferred strands were introduced into the fourth example flow cell in the same manner described in Example 1.

As the control, Illumina’s PCR-free DNA preparation was performed in a tube. The control sample was introduced into one of the flow cells and tagmentation was allowed to occur.

The DNA sample was prepared as described in Example 1 and was introduced into each of the first, second, third, and fourth flow cells and tagmentation was allowed to occur.

The insert size of the tagmented control DNA sample and the tagmented DNA samples using the first, second, third and fourth example flow cells were determined by sequencing and aligning the data using Illumina’s Dynamic Read Analysis for GENomics (DRAGEN) software. The results are shown in FIG. 8. As depicted, the removal of the 5′ phosphate from the non-transferred strands, alone or in combination with the addition of the 3′ fluorophore on the non-transferred strands, increased the insert size.

### Example 4

Flow cells including PAZAM and grafted P5 and P7 primers in the depressions were used.

The BCN-P5 and BCN-P7 transposome complexes (with 5′ phosphate non-transferred strands) from Example 1 were used as active transposome complexes.

Inactive BCN-P5 and BCN-P7 transposome complexes with 3′ dideoxycytosine on the transferred strands were also prepared as described in Example 1, except that the P5 primers had 3′ dideoxycytosine attached to the mosaic end.

The active BCN-P5 and BCN-P7 transposome complexes were introduced into different flow cells with different percentages of the inactive BCN-P5 and BCN-P7 transposome complexes. The percentage of the inactive BCN-P5 and BCN-P7 transposome complexes ranged from 20% to 60% at 10% increments.

The DNA sample was prepared as described in Example 1 and was introduced into each of the flow cells and tagmentation was allowed to occur.

The insert size of the DNA samples in the various flow cells were determined by sequencing and aligning the data using Illumina’s Dynamic Read Analysis for GENomics (DRAGEN) software. The results are shown in FIG. 9, where the samples are identified by the percentage of the inactive BCN-P5 and BCN-P7 transposome complexes in the flow cell. As depicted, the inclusion of the 3′ dideoxycytosine on the transferred strands increased the insert size. Moreover, the insert size increased with an increasing percentage of the inactive BCN-P5 and BCN-P7 transposome complexes.

### Example 5

25 µl of whole blood was added to a lysis tube containing an example of the inorganic salt free lysis buffer. The inorganic salt free lysis buffer consisted of 3.6 units of thermolabile Proteinase K (TLPK), 0.2% sodium dodecyl sulfate, 50 mM Tris HCI (pH 8), 1 mM EDTA, 0.5% TWEEN™ 20, and deionized water. Lysis was performed at 37° C. (using a heat block) for about 15 minutes to generate a crude lysate.

The temperature of the crude lysate was then raised to 55° C. for about 10 minutes to inactivate the TLPK.

A mixture of alphacyclodextrin and a tagmentation buffer (consisting of water, 5 mM magnesium acetate, and 10 mM Tris acetate salt, pH 7.6) were added to the lysis tube to generate a complexed crude lysate.

The complexed crude lysate was exposed to tagmentation on a flow cell including PAZAM, P5 and P7 primers grafted to the PAZAM, and BCN-P5 and BCN-P7 transposome complexes (with 5′ phosphate non-transferred strands) from Example 1 grafted to the PAZAM. The fragments resulting from tagmentation were amplified using the primers. The reverse strands were cleaved, and the forward strands were sequenced.

As a first control, Illumina’s PCR-free DNA preparation was performed in a tube to produce a DNA library. The DNA library was denatured using NaOH and Tris HCI, and was added to a flow cell including PAZAM and P5 and P7 primers grafted to the PAZAM. The DNA library was amplified to generate clusters in the depressions of the flow cell. The reverse strands were cleaved, and the forward strands were sequenced.

As a second control, DNA was used. The DNA was prepared in the same manner as the complexed crude lysate.

The insert size of the tagmented control DNA samples and the tagmented complexed crude lysate samples were determined by sequencing and aligning the data using Illumina’s Dynamic Read Analysis for GENomics (DRAGEN) software. The results are shown in FIG. 10. As depicted, the insert size of the tagmented complexed crude lysate was smaller with a larger bias for smaller fragments.

The sequencing metrics that are presented in FIG. 10 include the percentage of Q30 bases, the percentage of mapped reads, the coverage, and the percentage of reads passing filter.

Q30 is equivalent to the probability of an incorrect base call 1 in 1000 times. This means that the base call accuracy (i.e., the probability of a correct base call) is 99.9%. A lower base call accuracy of 99% (Q20) will have an incorrect base call probability of 1 in 100, meaning that every 100 base pair sequencing reads will likely contain an error. When sequencing quality reaches Q30, virtually all of the reads will be perfect, having zero errors and ambiguities.

Mapped read percentage refers to the percentage of reads that are aligned to the reference genome.

Coverage depth refers to the average number of sequencing reads that align to, or “cover,” each base in the sequenced sample. The Lander/Waterman equation is a method for calculating coverage (C) based on your read length (L), number of reads (N), and haploid genome length (G): C = LN / G.

A higher mapped read percentage and a higher coverage depth are indicative of the accuracy of the sequencing.

Passing filter (PF) is the metric used to describe clusters which pass a chastity threshold and are used for further processing and analysis of sequencing data. The %PF calculation involves the application of a chastity filter to each cluster. “Chastity” is defined as the ratio of the brightest base intensity divided by the sum of the brightest and second brightest base intensities. Clusters “pass filter” if no more than 1 base call has a chastity value below 0.6 in the first 25 cycles. This filtration process removes the least reliable clusters from the image analysis results. As such, a higher %passing filter (%PF) result indicates an increased yield of usable sequencing data.

The Q30 score, the mapped reads percentage, and the coverage for the complexed crude lysate samples were comparable to both of the control samples (although it is noted that all of the samples were downsampled). The %PF and the GC bias for the complexed crude lysate samples were comparable to the second control sample, while the first control sample was higher.

This example illustrates that a 30x human genome can be obtained from crude lysate on a flow cell using on board tagmentation, amplification, and sequencing.

### Example 6

This example was performed to assess the efficiency of betacyclodextrin in protecting two different polymerases from the effects of SDS during a polymerase chain reaction.

PCR was performed on a 350 base pair Lambda DNA target. The polymerases used were Q5® High-Fidelity DNA polymerase (from NEB) and a low fidelity polymerase. For each of the polymerases, sixteen samples were tested. Eight samples were tested without any SDS and with different concentrations (from 0% to 8%) of betacyclodextrin and eight samples were tested with 5 µL of 1% SDS (0.05% final concentration) and different concentrations (from 0% to 8%) of betacyclodextrin.

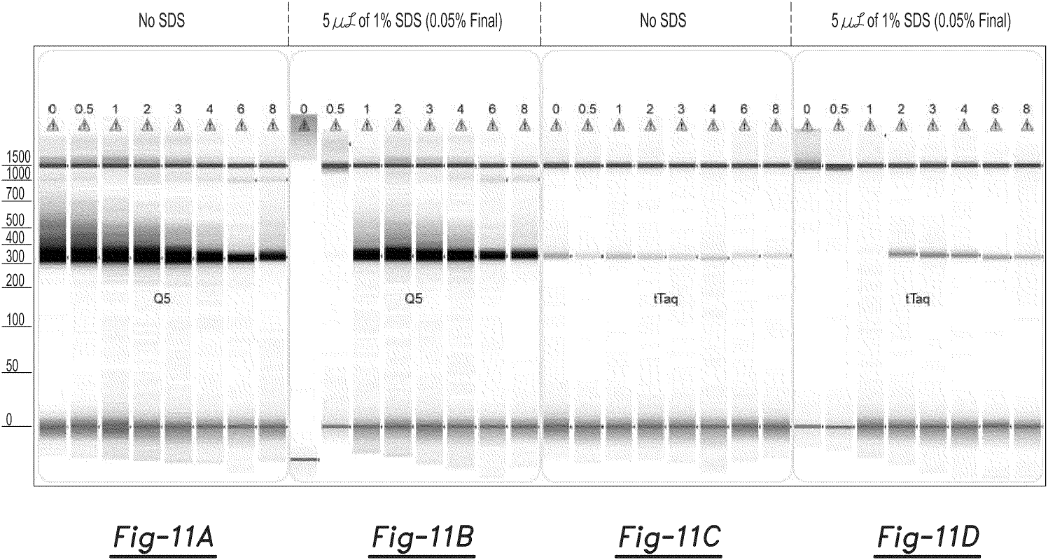

Sizing analysis of the PCR products was performed using an Agilent TapeStation® system and the D1000 ScreenTape® assay. Black and white reproductions of the gel images for i) the samples including the Q5® High-Fidelity DNA polymerase without any SDS, ii) the samples including the Q5® High-Fidelity DNA polymerase with SDS, iii) the samples including the low fidelity polymerase without any SDS, and iv) the samples including the low fidelity polymerase with SDS, are shown, respectively, in FIG. 11A, FIG. 11B, FIG. 11C, and FIG. 11D.

As depicted in FIG. 11A and FIG. 11C, amplification products are shown in all lanes when SDS was not used. The results in FIG. 11B and FIG. 11D indicate that when SDS was present at 0.05%, each of the polymerases was unable to amplify the DNA in the absence of betacyclodextrin or when a low level (0.5%) of betacyclodextrin was included. These results illustrate that SDS can deleteriously affect the polymerase activity. The results in FIG. 11B and FIG. 11D also indicate that when the level of betacyclodextrin was increased to about 1% (for the Q5® High-Fidelity DNA polymerase samples) and to about 2% (for the low fidelity polymerase), the polymerase activity was recovered. This clearly shows the sequestering effect that betacyclodextrin has on SDS. At higher betacyclodextrin concentrations (e.g., from about 6% to about 8%), amplification was somewhat inhibited. As such, an upper limit for this chaotropic detergent chelator may be 6% or 8%.

### Example 7

This example was performed to assess the efficiency of betacyclodextrin in protecting a gap fill ligation enzyme from the effects of SDS during Illumina’s PCR-free DNA preparation.

First control samples followed the protocol for the Illumina’s PCR-free DNA preparation. The DNA was exposed to the following workflow: 50 µL of tagmentation buffer → 10 µL 1% SDS → wash 1 time → 50 µL gap-fill ligation with a betacyclodextrin titration (concentration varied from 0% to 5%) → supernatant removed → addition of NaOH → addition of solid phase reversible immobilization (SPRI) beads to reversibly bind the DNA.

Second control samples followed a modified protocol for the Illumina’s PCR-free DNA preparation, with the reagent volumes adjusted. The DNA was exposed to the following workflow: 20 µL of tagmentation buffer → 5 µL 0.6% SDS → wash 1 time → 100 µL gap-fill ligation with a betacyclodextrin titration (concentration varied from 0% to 5%) → supernatant removed → addition of NaOH → addition of SPRI beads to reversibly bind the DNA.

Third samples followed a modified protocol for the Illumina’s PCR-free DNA preparation, with the wash step being completely removed. The DNA was exposed to the following workflow: 20 µL of tagmentation buffer → 5 µL 0.6% SDS → 100 µL gap-fill ligation with a betacyclodextrin titration (concentration varied from 0% to 5%) → supernatant removed → addition of NaOH → addition of SPRI beads to reversibly bind the DNA.

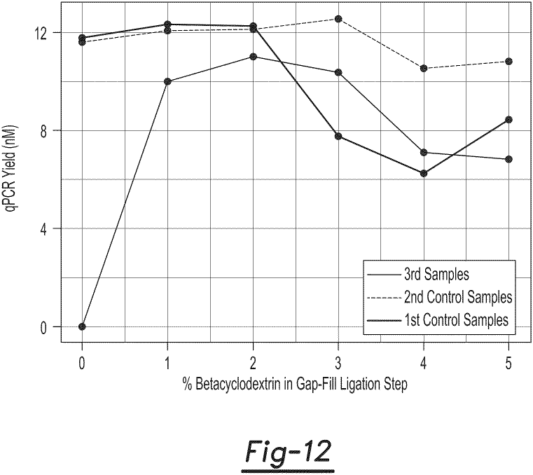

Each of the samples was exposed to quantitative PCR, and the results are shown in FIG. 12. The wash step in the first and second control samples was performed to remove both the SDS and the transposase used during tagmentation. This was performed to prevent inhibition of enzymes used in subsequent steps (e.g., the gap-fill ligation enzyme). The results for the third sample with no betacyclodextrin illustrate the strong negative impact of SDS when it is not washed away. The results for the third samples with 1%, 2%, and 3% betacyclodextrin illustrate that the betacyclodextrin counteracts the SDS. These results indicate that the addition of a small amount of betacyclodextrin enables the removal of the wash step without reintroducing the deleterious effects of the chaotropic agent and thus can help to streamline the library preparation process.

### Example 8

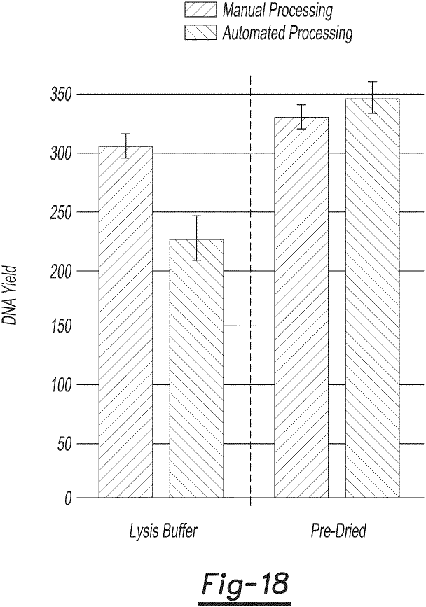

In this example, manual DNA extraction was performed and automated DNA extraction was performed using a HAMILTON® instrument. In both experiments, a Mitra tip was used for collection of a blood sample. In the first experiment, Proteinase K was added to a lysis buffer (from the Illumina DNA PCR-free kit), and in the second experiment, Proteinase K was pre-dried on the Mitra tip before blood collection. The DNA yield from each experiment was determined and the results are shown in FIG. 18. As depicted, the results for each processing type were comparable, but the pre-dried examples were particularly effective. It is believed that similar results may be achieved using zinc oxide nanoparticles in place of the Proteinase K.

### Example 9

Three different workflows were used in this example to compare the lysis of whole blood using different examples of the inorganic salt free lysis buffer and different protease inactivation techniques.

In the first workflow, 25 µl of whole blood was added to a lysis tube containing an example of the inorganic salt free lysis buffer. The inorganic salt free lysis buffer consisted of 3.6 units of thermolabile Proteinase K (TLPK), 0.2% sodium dodecyl sulfate, 50 mM Tris HCl (pH 8), 1 mM EDTA, 0.5% TWEEN™ 20, and deionized water. Lysis was performed at 37° C. (using a heat block) for about 15 minutes to generate a crude lysate. The temperature of the crude lysate was then raised to 55° C. for about 10 minutes to inactivate the TLPK. A mixture of alphacyclodextrin (2.7%) and a tagmentation buffer (consisting of water, 5 mM magnesium acetate, and 10 mM Tris acetate salt, pH 7.6) was added to the lysis tube to generate a first complexed crude lysate.

In the second workflow, 25 µl of whole blood was added to a lysis tube containing another example of the inorganic salt free lysis buffer. The inorganic salt free lysis buffer consisted of 0.8 mg/mL of Proteinase K (PK), 0.2% sodium dodecyl sulfate, 50 mM Tris HCl (pH 8), 1 mM EDTA, 0.5% TWEEN™ 20, and deionized water. Lysis was performed at 37° C. (using a heat block) for about 15 minutes to generate a crude lysate. The temperature of the crude lysate was then raised to 80° C. for about 10 minutes to about 15 minutes to inactivate the PK. A mixture of alphacyclodextrin (2.7%) and a tagmentation buffer (consisting of water, 5 mM magnesium acetate, and 10 mM Tris acetate salt, pH 7.6) was added to the lysis tube to generate a second complexed crude lysate.

In the third workflow, 25 µl of whole blood was added to a lysis tube containing another example of the inorganic salt free lysis buffer. The inorganic salt free lysis buffer consisted of 0.8 mg/mL of Proteinase K (PK), 0.2% sodium dodecyl sulfate, 50 mM Tris HCl (pH 8), 1 mM EDTA, 0.5% TWEEN™ 20, and deionized water. Lysis was performed at 37° C. (using a heat block) for about 15 minutes to generate a crude lysate. A mixture of alphacyclodextrin (2.7%), tetrapeptidyl chloromethyl ketone (0.08 mg/mL), and a tagmentation buffer (consisting of water, 5 mM magnesium acetate, and 10 mM Tris acetate salt, pH 7.6) was added to the lysis tube to inactivate the PK and to generate a third complexed crude lysate.

Each of the first, second and third complexed crude lysates was respectively exposed to tagmentation on a flow cell including PAZAM, P5 and P7 primers grafted to the PAZAM, and BCN-P5 and BCN-P7 transposome complexes (with 5′ phosphate non-transferred strands) from Example 1 grafted to the PAZAM. The fragments resulting from tagmentation were amplified using the primers. The reverse strands were cleaved, and the forward strands were sequenced.

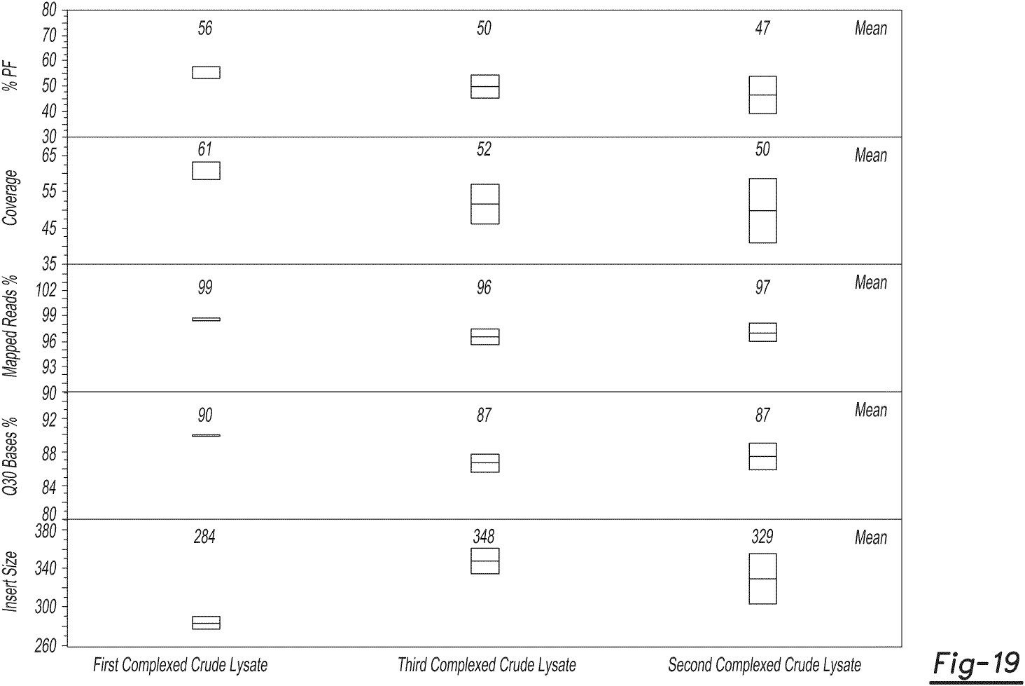

The insert size and sequencing metrics (i.e., percentage of Q30 bases, the percentage of mapped reads, the coverage, and the percentage of reads passing filter) were determined as described in Example 5 (although the samples were not downsampled). The results are shown in FIG. 19. As depicted in FIG. 19, the insert size of the first complexed crude lysate was slightly less than the second and third complexed crude lysates. Overall, each of the workflows, and its lysis buffer and inactivation technique, e.g., heat or TCK as an inhibitor, generated suitable insert sizes and yielded similar sequencing metrics. This example again illustrates that a greater than 30x human genome can be obtained from crude lysate on a flow cell using on board tagmentation, amplification, and sequencing.

### Example 10

Some of the reagents (streptavidin, lysis buffer, tagmentation buffer, and transposome buffer) described herein were lyophilized in order to determine the effects of lyophilization relative to reagents that were not lyophilized.

The streptavidin reagent was prepared with 2 µM streptavidin, 100 mM Tris (pH 7.5), 60 mM NaCl, 0.2% TWEEN™ 20, 540 mM Trehalose, and deionized water.

The inorganic salt free lysis buffer was prepared with 0.056 mM of Proteinase K (PK), 0.4% sodium dodecyl sulfate, 100 mM Tris HCl (pH 8), 2 mM EDTA, 1% TWEEN™ 20, 440 mM Trehalose, and deionized water.

The tagmentation buffer was prepared with 10 mM magnesium acetate, 20 mM Tris acetate salt (pH 7.6), 571 mM Trehalose, and deionized water.

Transposome buffers were prepared with the asymmetric transposome complexes. P7 transposome complexes included biotin at the 3′ end of the non-transferred strand and P5 tranposome complexes included biotin at the 5′ end of the transferred strand. The final transposome concentrations were 0.4 µM and 0.5 µM (with 80% of the P7 transposome complexes and 20% of the P5 transposome complexes), respectively. The 0.4 µM transposome buffer included 0.64% glycerol, 63.36 mM NaCl, 101.68 mM Tris (pH 7.5), 0.034 mM DTT, 0.003% TRITON® X-100, 0.003 mM EDTA, 0.2% TWEEN™ 20, and 18% Trehalose. The 0.5 µM transposome buffer included 0.8% glycerol, 64.2 mM NaCl, 102.1 mM Tris (pH 7.5), 0.042 mM DTT, 0.004% TRITON® X-100, 0.004 mM EDTA, 0.2% TWEEN™ 20, and 18% Treehalose.

For each reagent, a liquid sample was prepared and a lyophilized sample was prepared. Lyophilization was performed at a temperature ranging from about -25° C. to about -50° C. and at a pressure ranging from about 30 mPa to about 90 mPa. The weight average molecular weight and/or concentration of the reagent components in the resulting microspheres or single block (cake format) are shown in Tables 1-4.

TABLE 1

| Reagent Component | Streptavidin Microspheres | |
|---|---|---|
| | MW | Mg/mL |
| Streptavidin | 66000 | 0.13 |
| Tris (pH 7.5) | 121.14 | 12.11 |
| NaCl | 58.44 | 3.51 |
| TWEEN™ 20 | 122.54 | 0.002 |
| Trehalose | 342.3 | 185 |
| Total Mass | | 200.75 |
| Total Solute Content | | 20.10% |

TABLE 2

| Reagent Component | Lysis Block | |
|---|---|---|
| | MW | Mg/mL |
| Proteinase K | 28907 | 1.62 |
| Tris HCl (pH 7.5) | 121.14 | 12.11 |
| SDS | 288.38 | 10.00 |
| EDTA | 292.24 | 0.59 |
| TWEEN™ 20 | 1227.54 | 25.00 |
| Trehalose | 342.30 | 150.61 |
| Total Mass | | 199.93 |
| Total Solute Content | | 19.99% |

TABLE 3

| Reagent Component | Tagmentation Microspheres | |
|---|---|---|
| | MW | Mg/mL |
| Tris Acetate | 181.19 | 2.43 |
| Magnesium Acetate | 142.39 | 2.67 |
| Trehalose | 342.30 | 196 |
| Total Mass | | 201 |
| Total Solute Content | | 20.1 |

TABLE 4

| Reagent Component | 0.4 µM Transposome Block | 0.5 µM Transposome Block |
|---|---|---|
| | Mg/200 mL | Mg/200 mL |
| Glycerol | 1.28 | 1.6 |
| NaCl | 0.74055168 | 0.7503696 |
| Tris (pH 7.5) | 2.46350304 | 2.4736788 |
| DTT | 0.002382173 | 0.002977716 |
| TRITON® X-100 | 0.00672 | 0.0084 |
| EDTA | 0.000196385 | 0.000245482 |
| TWEEN™ 20 | 0.4 | 0.4 |
| Trehalose | 35 | 35 |

Each of the lyophilized samples (microspheres or blocks) were reconstituted with water.

### Streptavidin Results

The liquid and reconstituted lyophilized streptavidin samples were loaded onto respective flow cells including biotinylated PAZAM (where biotin was attached to PAZAM through BCN linkers) and P5 and P7 primers grafted to the PAZAM with biotin-P5 and biotin-P7 transposome complexes. Wash solutions were flowed through the flow cells to remove unbound streptavidin and transposome complexes. DNA samples (500 ng) in a tagmentation buffer were then introduced into the respective flow cells. The fragments resulting from tagmentation were amplified using the primers. The reverse strands were cleaved, and the forward strands were sequenced.

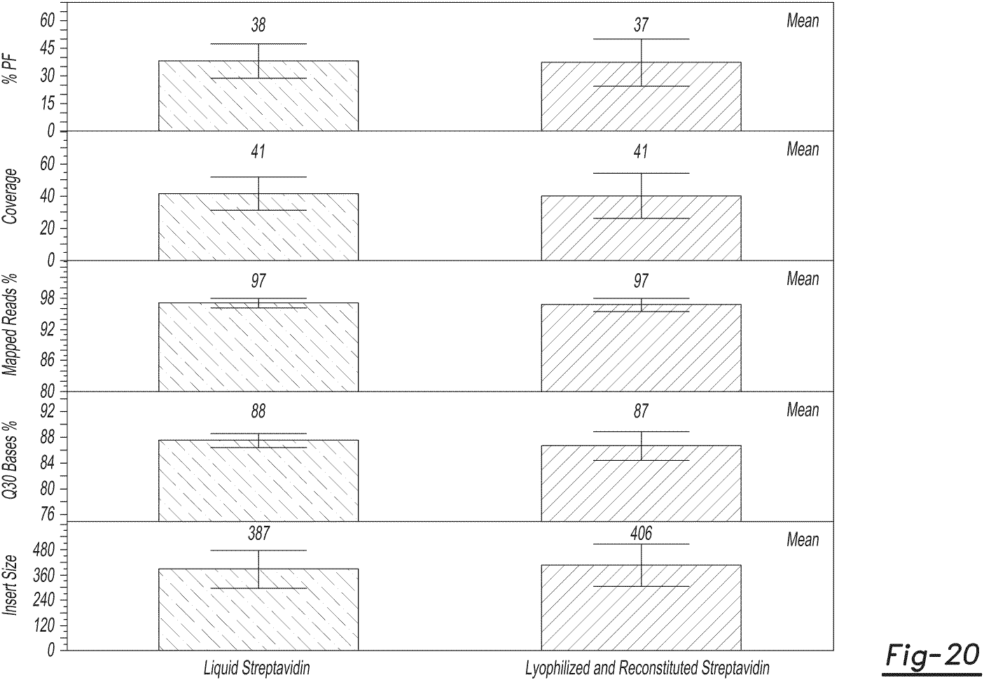

The insert size and sequencing metrics (i.e., percentage of Q30 bases, the percentage of mapped reads, the coverage, and the percentage of reads passing filter) were determined as described in Example 5 (although the samples were not downsampled). The insert size and sequencing metrics for the DNA analyzed on the flow cells prepared with the liquid and reconstituted lyophilized streptavidin samples are shown in FIG. 20. As depicted, the use of lyophilized and reconstituted streptavidin to attach the transposome complexes had no negative impact on the downstream DNA processing in terms of insert size or sequencing metrics.

### Lysis Buffer Results

25 µl of whole blood was added to each of the liquid and reconstituted lyophilized lysis buffer samples. Lysis was performed at 37° C. (using a heat block) for about 15 minutes to generate respective crude lysates. The temperature of the crude lysates was then raised to 80° C. for about 10 minutes to about 15 minutes to inactivate the PK. A mixture of alphacyclodextrin (2.7%) and a tagmentation buffer (consisting of water, 5 mM magnesium acetate, and 10 mM Tris acetate salt, pH 7.6) was added to the lysis tube to generate complexed crude lysates. Each of the complexed crude lysates (respectively formed with the liquid lysis buffer and the reconstituted lyophilized lysis buffer) was respectively exposed to tagmentation on a flow cell including PAZAM, P5 and P7 primers grafted to the PAZAM, and BCN-P5 and BCN-P7 transposome complexes (with 5′ phosphate non-transferred strands) from Example 1 grafted to the PAZAM. The fragments resulting from tagmentation were amplified using the primers. The reverse strands were cleaved, and the forward strands were sequenced.

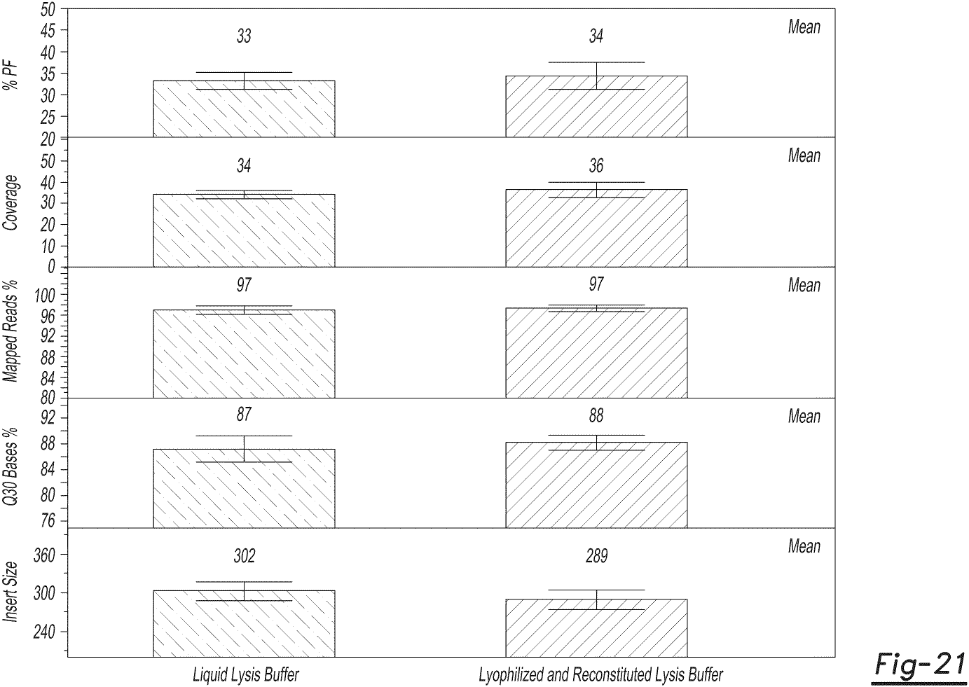

The insert size and sequencing metrics (i.e., percentage of Q30 bases, the percentage of mapped reads, the coverage, and the percentage of reads passing filter) were determined as described in Example 5 (although the samples were not downsampled). The results are shown in FIG. 21. As depicted, the use of lyophilized and reconstituted lysis buffer to generate crude lysate from whole blood samples had no negative impact on the downstream DNA processing in terms of insert size or sequencing metrics.

### Tagmentation Buffer Results

DNA samples (500 ng) were respectively introduced into the liquid and reconstituted lyophilized tagmentation buffers. These samples were then introduced into respective flow cells including PAZAM, P5 and P7 primers grafted to the PAZAM, and BCN-P5 and BCN-P7 transposome complexes (with 5′ phosphate non-transferred strands) from Example 1 grafted to the PAZAM. The fragments resulting from tagmentation were amplified using the primers. The reverse strands were cleaved, and the forward strands were sequenced.

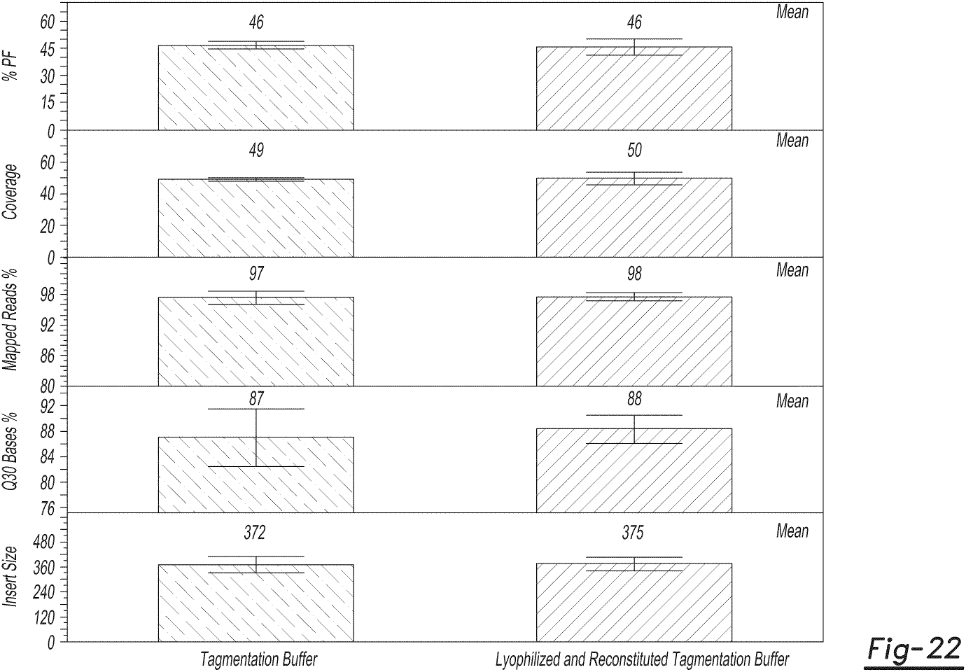

The insert size and sequencing metrics (i.e., percentage of Q30 bases, the percentage of mapped reads, the coverage, and the percentage of reads passing filter) were determined as described in Example 5 (although the samples were not downsampled). The results are shown in FIG. 22. As depicted, the use of a lyophilized and reconstituted tagmentation buffer during tagmentation had no negative impact on the downstream DNA processing in terms of insert size or sequencing metrics.

The results in this example illustrate that lyophilization may be used for various reagents used in flow cell preparation and sample preparation without deleteriously affecting downstream sequencing.

### Transposome Results

The liquid and reconstituted lyophilized transposome buffer samples were loaded onto respective flow cells including biotinylated PAZAM (where biotin was attached to PAZAM through BCN linkers) and P5 and P7 primers grafted to the PAZAM. Wash solutions were flowed through the flow cells to remove unbound streptavidin and transposome complexes. DNA samples (500 ng) in a tagmentation buffer were then introduced into the respective flow cells. The fragments resulting from tagmentation were amplified using the primers. The reverse strands were cleaved, and the forward strands were sequenced.

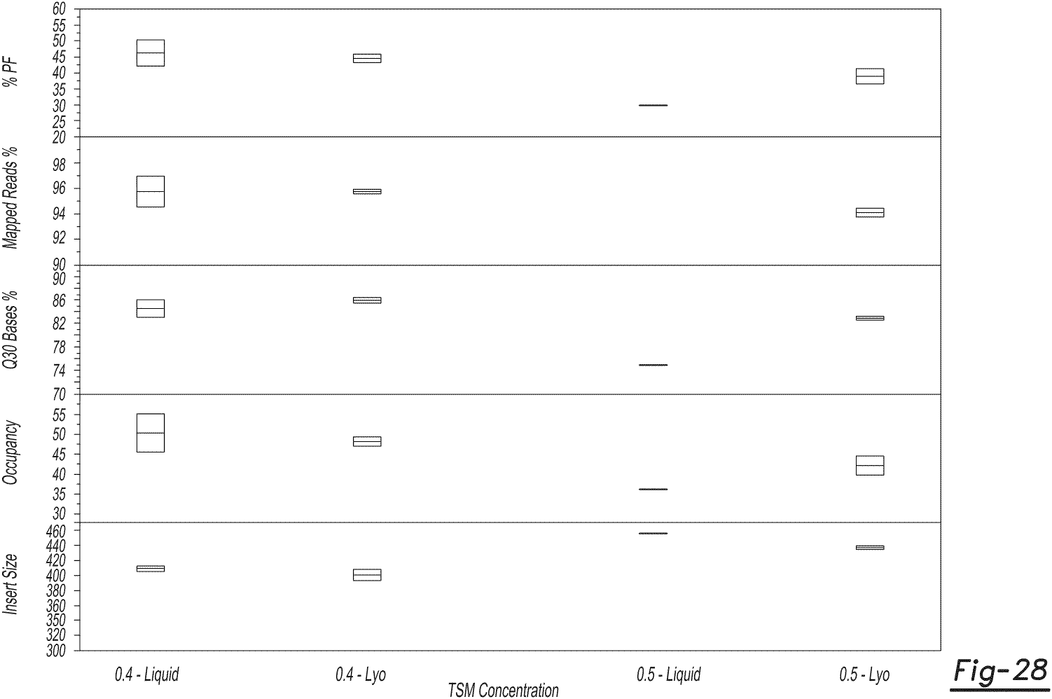

The insert size and some of the sequencing metrics (i.e., percentage of Q30 bases, the percentage of mapped reads, and the percentage of reads passing filter) were determined as described in Example 5 (although the samples were not downsampled). In this example, occupancy or %occupied was also determined. As used herein, “occupany or %occupied” refers to the percentage of depressions that contain a cluster versus those that are empty. The insert size and sequencing metrics for the DNA analyzed on the flow cells prepared with the liquid and reconstituted lyophilized streptavidin samples are shown in FIG. 28. As depicted, the use of lyophilized and reconstituted transposomes at different concentrationss had no negative impact on the downstream DNA processing in terms of insert size or sequencing metrics.

### Example 11

As a control, Illumina’s PCR-free DNA preparation (NEXTSEQ™ 2 K protocol) was performed in a tube to produce a gDNA library. The gDNA library was denatured using NaOH and Tris HCl and was added to a flow cell including PAZAM and P5 and P7 primers grafted to the PAZAM. The gDNA library was amplified to generate clusters in the depressions of the flow cell. The reverse strands were cleaved, and the forward strands were sequenced.

For the examples, flow cells including biotinylated PAZAM (where streptavidin was attached to biotin, and the biotin was attached to PAZAM through BCN linkers) and grafted P15 and P7 primers in the depressions were used. Biotin-P15 and biotin-P7 transposomes were incorporated into a buffer (50 mM Tris (pH 7.5), 30 mM NaCl, 0.1% Tween-20, and water), and were grafted to the biotinylated PAZAM through streptavidin. The flow cells were washed with a high salt buffer. The transposome mixture was added to each flow cell and incubated for about 30 minutes at room temperature. After the desired incubation period, the control flow cells were flushed with a buffer.

For one example, 500 ng gDNA was incorporated into a tagmentation buffer (consisting of water, 5 mM magnesium acetate, and 10 mM Tris acetate salt, pH 7.6). The gDNA was exposed to tagmentation on one of the flow cells. The fragments resulting from tagmentation were amplified using the primers. The reverse strands were cleaved, and the forward strands were sequenced.

For another example, 300 µL of a complexed crude lysate was used. Whole blood was added to a lysis tube containing an example of the inorganic salt free lysis buffer. The inorganic salt free lysis buffer consisted of 3.6 units of thermolabile Proteinase K (TLPK), 0.2% sodium dodecyl sulfate, 50 mM Tris HCl (pH 8), 1 mM EDTA, 0.5% TWEEN™ 20, and deionized water. Lysis was performed at 37° C. (using a heat block) for about 15 minutes to generate a crude lysate. The temperature of the crude lysate was then raised to 55° C. for about 10 minutes to inactivate the TLPK. A mixture of alphacyclodextrin (2.7%) and a tagmentation buffer (consisting of water, 5 mM magnesium acetate, and 10 mM Tris acetate salt, pH 7.6) was added to the lysis tube to generate a complexed crude lysate. The complexed crude lysate was exposed to tagmentation on another one of the flow cells. The fragments resulting from tagmentation were amplified using the primers. The reverse strands were cleaved, and the forward strands were sequenced.

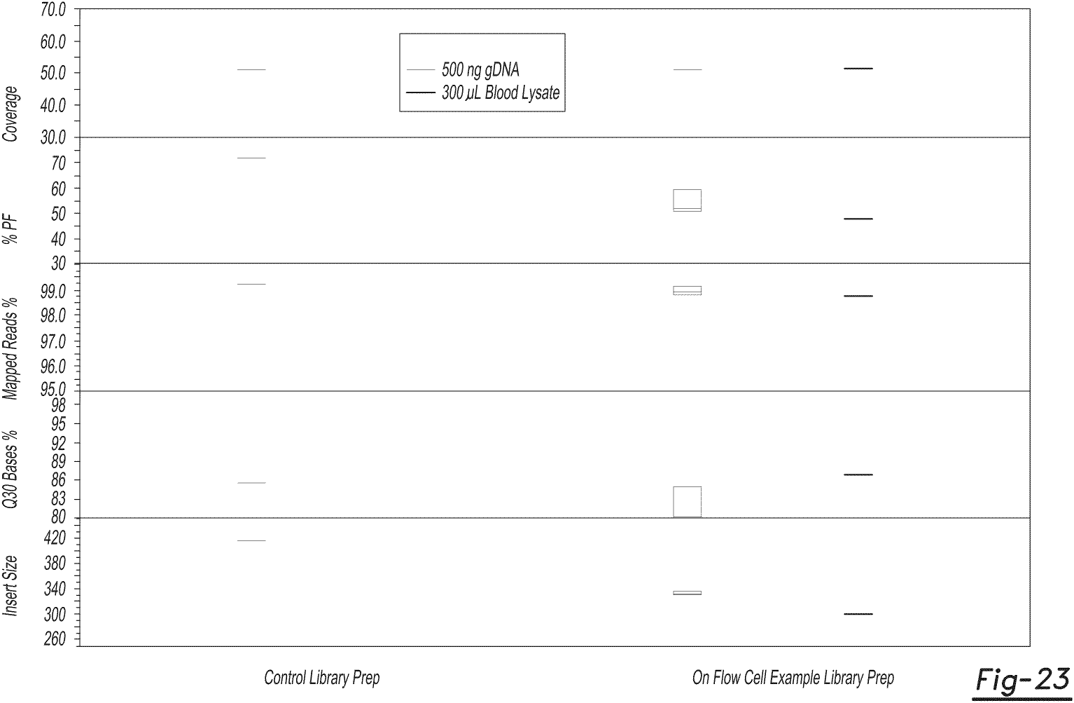

The insert size and sequencing metrics (i.e., percentage of Q30 bases, the percentage of mapped reads, the coverage, and the percentage of reads passing filter) were determined for the control and the examples as described in Example 5 (the samples were downsampled). The results are shown in FIG. 23. As depicted in FIG. 23, the insert size of both the example gDNA and the crude lysate were slightly less than the control gDNA. Overall, the sequencing metrics were comparable to the control.

### Example 12

This example compared two different methods for preparing the flow cell surface with BCN-biotin and streptavidin for subsequent introdution of biotinylated transposome complexes.

In one example, the BCN-biotin was added to a flow cell including PAZAM in the depressions. The BCN-biotin-streptavidin was incubated at 60° C. for about 2 hours. Streptavidin was then added and incubated for 30 minutes at room temperature.

In another example, BCN-biotin (20 µL) and streptavidin (20 µL) were preincubated for 30 minutes in 20 µL of the buffer (50 mM Tris (pH 7.5), 30 mM NaCl, 0.1% Tween-20, and water). After 30 minutes, the mixture as diluted in 140 µL of an Na₂SO₄ buffer. The diluted BCN-biotin-streptavidin was then added to a flow cell including PAZAM in the depressions, and was incubated at 60° C. for about 2 hours.

500 ng gDNA was incorporated into a tagmentation buffer (consisting of water, 5 mM magnesium acetate, and 10 mM Tris acetate salt, pH 7.6). Respective gDNA samples were exposed to tagmentation on each of the flow cells. The fragments resulting from tagmentation were amplified using the primers. The reverse strands were cleaved, and the forward strands were sequenced.

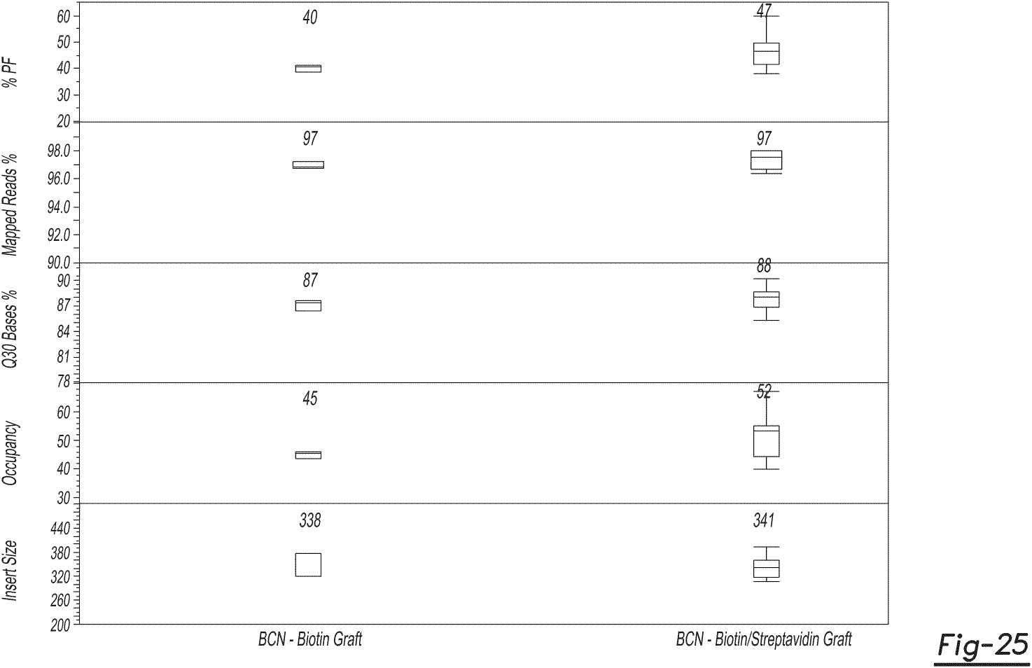

The insert size and sequencing metrics (i.e., percentage of Q30 bases, the percentage of mapped reads, the occupancy, and the percentage of reads passing filter) were determined for the examples as described in Examples 5 and 10. The results are shown in FIG. 25. As depicted in FIG. 25, either of the methods for introducing BCN-biotin-streptavidin may be used without deleteriously affecting the downstream metrics.

### Additional Notes

In any of the examples disclosed herein, the substrate may include a single lane defined therein rather than multiple depressions. Within the lane, the polymeric hydrogel may be selectively deposited to form individual pads. The pads are similar to the polymeric hydrogel within the depressions, and can be used in any of the example disclosed herein.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

While several examples have been described in detail, it is to be understood that the disclosed examples may be modified. Therefore, the foregoing description is to be considered non-limiting.

What is claimed is:

1. A flow cell, comprising:
a substrate having depressions separated by interstitial regions;
first and second primers immobilized within each of the depressions;
first transposome complexes immobilized within each of the depressions, the first transposome complexes including a first amplification domain; and
second transposome complexes immobilized within each of the depressions, the second transposome complexes including a second amplification domain;
wherein i) some of the first transposome complexes, or ii) some of the second transposome complexes, or iii) some of both of the first and second transposome complexes include a modification to reduce tagmentation efficiency.

2. The flow cell as defined in claim 1, wherein the modification to reduce tagmentation efficiency is exclusion of a phosphate group at a 5′ end of a non-transferred strand of i) the some of the first transposome complexes, or ii) the some of the second transposome complexes, or iii) the some of both of the first and second transposome complexes.

3. The flow cell as defined in claim 1, wherein the modification to reduce tagmentation efficiency is a fluorophore attached at a 5′ end of a non-transferred strand of i) the some of the first transposome complexes, or ii) the some of the second transposome complexes, or iii) the some of both of the first and second transposome complexes.

4. The flow cell as defined in claim 1, wherein the modification to reduce tagmentation efficiency is a fluorophore attached at a 3′ end of a non-transferred strand of i) the some of the first transposome complexes, or ii) the some of the second transposome complexes, or iii) the some of both of the first and second transposome complexes.

5. The flow cell as defined in claim 1, wherein the modification to reduce tagmentation efficiency is a dideoxycytosine, a thymine, or a cytosine attached at a 3′ end of a transferred strand of i) the some of the first transposome complexes, or ii) the some of the second transposome complexes, or iii) the some of both of the first and second transposome complexes.

6. A flow cell, comprising:
a substrate having depressions separated by interstitial regions;
first and second primers immobilized within each of the depressions;
first transposome complexes including a first amplification domain;
second transposome complexes including a second amplification domain;
wherein one of:
i) the first and second transposome complexes are respectively immobilized at different regions of each of the depressions; or
ii) the first and second transposome complexes are respectively immobilized within each of the depressions and on each of the interstitial regions; or
iii) the first and second transposome complexes are respectively immobilized on different areas of the interstitial regions.

7. The flow cell as defined in claim 6 wherein each of first and second transposome complexes respectively includes:
a transposon end; and
a transposase enzyme non-covalently bound to the transposon end.

8. A method for increasing an insert size of a deoxyribonucleic acid sample, comprising:
introducing the DNA sample to a flow cell including:
depressions separated by interstitial regions;
first and second primers immobilized within each of the depressions;
first transposome complexes immobilized within each of the depressions or on the interstitial regions, the first transposome complexes including a first amplification domain; and
a second transposome complex immobilized within each of the depressions or on the interstitial regions, the second transposome complexes including a second amplification domain;
introducing a condensation agent or a tagmentation inhibitor with the DNA sample, the condensation agent being selected from the group consisting of a polycationic amine, polyethylene glycol, and a histone or the tagmentation inhibitor being selected from the group consisting a cation, a pH adjustor, and a chromatin assembly kit; and
incubating the DNA sample in the flow cell in the presence of the condensation agent or the tagmentation inhibitor, whereby tagmentation of the DNA sample takes place at some of the first transposome complexes and the second transposome complexes.

9. The method as defined in claim 8, wherein after tagmentation, the method further comprises:
introducing a washing solution into the flow cell;
heating the flow cell, containing the washing solution, to about 60° C.; and
then introducing an extension amplification mix into the flow cell.

10. A method, comprising:
introducing transposome complexes to a buffer solution, the transposome complexes including:
a transposon end including a portion of transferred strand hybridized to a portion of non-transferred strand, the transferred strand including a 5′ end alkyne functional group; and
a transposase enzyme non-covalently bound to the transposon end;
introducing the buffer solution containing the transposome complexes to a flow cell including depressions separated by interstitial regions, the depressions having therein a polymeric hydrogel with terminal azide or tetrazine groups; and
incubating the buffer solution in the flow cell, whereby the 5′ end alkyne functional groups of at least some of the transposome complexes respectively attach to at least some of the terminal azide or tetrazine groups.

11. The method as defined in claim 10, wherein the 5′ end alkyne functional group is bicyclo[6.1.0]nonyne.

12. The method as defined in claim 11, wherein:
the buffer solution is sodium sulfate; and
incubating is performed at about 37° C. for about 2 hours.

13. A method, comprising:
introducing transposome complexes to a flow cell, wherein:
the transposome complexes include:
a transposon end including a portion of a transferred strand hybridized to a portion of a non-transferred strand, the transferred strand including a 5′ biotinylated end; and
a transposase enzyme non-covalently bound to the transposon end;
the flow cell includes:
depressions separated by interstitial regions; and
a biotinylated polymeric hydrogel in the depressions;
introducing streptavidin to the flow cell; and
incubating the transposome complexes and the streptavidin in the flow cell, whereby the streptavidin respectively attaches the 5′ biotinylated end of at least some of the transposome complexes to the biotinylated polymeric hydrogel.

14. The method as defined in claim 13, further comprising preparing the biotinylated polymer hydrogel by grafting bicyclo[6.1.0]nonyne-biotin to terminal azide or tetrazine groups of a polymeric hydrogel.

15. The method as defined in claim 13, wherein the streptavidin is pre-attached to the 5′ biotinylated end of each transposome complex and thus is introduced as part of each transposome complex.

16. The method as defined in claim 13, wherein the streptavidin is introduced to the flow cell before the transposome complexes are introduced into the flow cell.

17. The method as defined in claim 13, wherein:
the transposome complexes, or the streptavidin, or both the transposome complexes and the streptavidin are dried; and
the method further comprises:
reconstituting the transposome complexes in a buffer before introducing the transposome complexes to the flow cell; or
reconstituting the streptavidin in a buffer before introducing the streptavidin to the flow cell; or
respectively reconstituting each of the transposome complexes and the streptavidin in respective buffers before introducing them into the flow cell.

18. A reusable flow cell, comprising:
a substrate including depressions separated by interstitial regions;
a biotinylated polymeric hydrogel in the depressions;
first and second biotinylated primers immobilized within each of the depressions;
first biotinylated transposome complexes immobilized within each of the depressions, the first biotinylated transposome complexes including a first amplification domain; and
second biotinylated transposome complexes immobilized within each of the depressions, the second biotinylated transposome complexes including a second amplification domain.

19. The reusable flow cell as defined in claim 18, wherein the first and second biotinylated primers are immobilized to the biotinylated polymeric hydrogel through streptavidin.

20. The reusable flow cell as defined in claim 18, wherein:
the first biotinylated transposome complexes are immobilized to the biotinylated polymeric hydrogel through streptavidin; and
the second biotinylated transposome complexes are immobilized to the biotinylated polymeric hydrogel through streptavidin.

21-91. (canceled)


SEQUENCE LISTING (10 sequences)

SEQ ID NO: 1 - DNA, 29 bp, synthetic construct
  source 1..29: mol_type = other DNA
  modified_base 23: mod_base = OTHER; note = uracil
aatgatacggcgaccaccgagatctacac

SEQ ID NO: 2 - DNA, 29 bp, synthetic construct
  source 1..29: mol_type = other DNA
  modified_base 23: mod_base = OTHER; note = alkene-thymidine
aatgatacggcgaccaccgaganctacac

SEQ ID NO: 3 - DNA, 24 bp, synthetic construct
  source 1..24: mol_type = other DNA
  modified_base 22: mod_base = OTHER; note = 8-oxoguanine
caagcagaagacggcatacganat

SEQ ID NO: 4 - DNA, 24 bp, synthetic construct
  source 1..24: mol_type = other DNA
  modified_base 20: mod_base = OTHER; note = 8-oxoguanine
caagcagaagacggcatacnagat

SEQ ID NO: 5 - DNA, 29 bp, synthetic construct
  source 1..29: mol_type = other DNA
  modified_base 23: mod_base = OTHER; note = allyl-T
aatgatacggcgaccaccgaganctacac

SEQ ID NO: 6 - DNA, 35 bp, synthetic construct
  source 1..35: mol_type = other DNA
gctggcacgtccgaacgcttcgttaatccgttgag

SEQ ID NO: 7 - DNA, 35 bp, synthetic construct
  source 1..35: mol_type = other DNA
cgtcgtctgccatggcgcttcggtggatatgaact

SEQ ID NO: 8 - DNA, 35 bp, synthetic construct
  source 1..35: mol_type = other DNA
acggccgctaatatcaacgcgtcgaatccgcaact

SEQ ID NO: 9 - DNA, 34 bp, synthetic construct
  source 1..34: mol_type = other DNA
gccgcgttacgttagccggactattcgatgcagc

SEQ ID NO: 10 - DNA, 24 bp, synthetic construct
  source 1..24: mol_type = other DNA
aggaggaggaggaggaggaggagg